SEQUENCE LISTING (81 sequences)

SEQ ID NO: 1 - protein, 118 aa, synthetic construct
  REGION 1..118: note = Synthetic Sequence_1923Ab2 VH
QVQLVESGGGVVQPGRSLRLSCAASGFTFRNYGMHWVRQAPGKGLEWVAVIWFDGINKYYADSVKGRFTISRDNSENTLYLQMNSLRAEDTAVYYCARDRSSSPFDFWGQGTLVTVSS

SEQ ID NO: 2 - protein, 107 aa, synthetic construct
  REGION 1..107: note = Synthetic Sequence_1923Ab2 VL
EIVMTQSPATLSLSPGERATLSCRASQNFSNNYLSWYQQTPGQAPRLLIYGASTRATGIPARFSGSGSGTDFTLTISSLQPEDFAVYYCQQDYNLFTFGPGTKVDIK

SEQ ID NO: 3 - protein, 119 aa, synthetic construct
  REGION 1..119: note = Synthetic Sequence_1923Ab3 VH
QVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSS

SEQ ID NO: 4 - protein, 108 aa, synthetic construct
  REGION 1..108: note = Synthetic Sequence_1923Ab3 VL
EIVMAQSPATLSLSPGERATLSCRASQSVSSSYLSWFQQKPGQAPRLLIYGASTRATGIPARFGGSGSGTDFTLTISSLQPEDFAVYYCQQDYNLPYTFGQGTKLEIK

SEQ ID NO: 5 - protein, 5 aa, synthetic construct
  REGION 1..5: note = Synthetic Sequence_1923Ab4 VH
NYGMH

SEQ ID NO: 6 - protein, 17 aa, synthetic construct
  REGION 1..17: note = Synthetic Sequence_1923Ab2 VH
VIWFDGINKYYADSVKG

SEQ ID NO: 7 - protein, 9 aa, synthetic construct
  REGION 1..9: note = Synthetic Sequence_1923Ab2 VH
DRSSSPFDF

SEQ ID NO: 8 - protein, 12 aa, synthetic construct
  REGION 1..12: note = Synthetic Sequence_1923Ab2 VL
RASQNFSNNYLS

SEQ ID NO: 9 - protein, 7 aa, synthetic construct
  REGION 1..7: note = Synthetic Sequence_1923Ab2 VL
GASTRAT

SEQ ID NO: 10 - protein, 8 aa, synthetic construct
  REGION 1..8: note = Synthetic Sequence_1923Ab2 VL
QQDYNLFT

SEQ ID NO: 11 - protein, 5 aa, synthetic construct
  REGION 1..5: note = Synthetic Sequence_1923Ab3 VH
SYDIN

SEQ ID NO: 12 - protein, 17 aa, synthetic construct
  REGION 1..17: note = Synthetic Sequence_1923Ab3 VH
WMNPNSGITGYAQRFQG

SEQ ID NO: 13 - protein, 10 aa, synthetic construct
  REGION 1..10: note = Synthetic Sequence_1923Ab3 VH
HRWGKGYLDL

SEQ ID NO: 14 - protein, 12 aa, synthetic construct
  REGION 1..12: note = Synthetic Sequence_1923Ab3 VL
RASQSVSSSYLS

SEQ ID NO: 15 - protein, 9 aa, synthetic construct
  REGION 1..9: note = Synthetic Sequence_1923Ab3 VL
QQDYNLPYT

SEQ ID NO: 16 - protein, 127 aa, synthetic construct
  REGION 1..127: note = Synthetic Sequence_1923Ab4 VH
QVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSS

SEQ ID NO: 17 - protein, 107 aa, synthetic construct
  REGION 1..107: note = Synthetic Sequence_1923Ab4 VL
SYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVL

SEQ ID NO: 18 - protein, 120 aa, synthetic construct
  REGION 1..120: note = Synthetic Sequence_1923Ab5 VH
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARERWQWLVLFDYWGQGTLVTVSS

SEQ ID NO: 19 - protein, 108 aa, synthetic construct
  REGION 1..108: note = Synthetic Sequence_1923Ab5 VL
EIVLTQSPATLSLSPGERATLSCRASQTITSSYLSWYQQKPGQAPRLLIYGASTRATGIPARFSGSGSGTDFTLTISSLQPEDFALYFCQQDFNLPYTFGQGTKLEIK

SEQ ID NO: 20 - protein, 122 aa, synthetic construct
  REGION 1..122: note = Synthetic Sequence_1923Ab6 VH
EVHLLESGGGLVQPGGSLRLSCAASGFTFSSYAMGWVRQAPGKGLEWVSIIGGGGGSTYYADSVKGRFTISRDNSKNTLYLQMNSLRAEDTAVYYCAKVGYSGTYGGWFDPWGQGTLVTVSS

SEQ ID NO: 21 - protein, 107 aa, synthetic construct
  REGION 1..107: note = Synthetic Sequence_1923Ab6 VL
DIQMTQSPSSLSASIGDRVAITCRASQGISNYLAWFQQKPGKAPKSLIYAASSLQSGVPSKFSGSGSGTDFTLTISSLQPEDFATYYCQQYNSYPLTFGGGTKVEIK

SEQ ID NO: 22 - protein, 17 aa, synthetic construct
  REGION 1..17: note = Synthetic Sequence_1923Ab4 VH
IIWYDGINKYYADSVKG

SEQ ID NO: 23 - protein, 18 aa, synthetic construct
  REGION 1..18: note = Synthetic Sequence_1923Ab4 VH
DQDYDILTGYYKEDYFDF

SEQ ID NO: 24 - protein, 11 aa, synthetic construct
  REGION 1..11: note = Synthetic Sequence_1923Ab4 VL
SGDALPKKYAY

SEQ ID NO: 25 - protein, 7 aa, synthetic construct
  REGION 1..7: note = Synthetic Sequence_1923Ab4 VL
EDSKRPS

SEQ ID NO: 26 - protein, 10 aa, synthetic construct
  REGION 1..10: note = Synthetic Sequence_1923Ab4 VL
YSTDSSGNRV

SEQ ID NO: 27 - protein, 5 aa, synthetic construct
  REGION 1..5: note = Synthetic Sequence_1923Ab5 VH
SYGMH

SEQ ID NO: 28 - protein, 17 aa, synthetic construct
  REGION 1..17: note = Synthetic Sequence_1923Ab5 VH
VIWYDGSNEYYADSVKG

SEQ ID NO: 29 - protein, 11 aa, synthetic construct
  REGION 1..11: note = Synthetic Sequence_1923Ab5 VH
ERWQWLVLFDY

SEQ ID NO: 30 - protein, 12 aa, synthetic construct
  REGION 1..12: note = Synthetic Sequence_1923Ab5 VL
RASQTITSSYLS

SEQ ID NO: 31 - protein, 9 aa, synthetic construct
  REGION 1..9: note = Synthetic Sequence_1923Ab5 VL
QQDFNLPYT

SEQ ID NO: 32 - protein, 5 aa, synthetic construct
  REGION 1..5: note = Synthetic Sequence_1923Ab6 VH
SYAMG

SEQ ID NO: 33 - protein, 17 aa, synthetic construct
  REGION 1..17: note = Synthetic Sequence_1923Ab6 VH
IIGGGGGSTYYADSVKG

SEQ ID NO: 34 - protein, 13 aa, synthetic construct
  REGION 1..13: note = Synthetic Sequence_1923Ab6 VH
VGYSGTYGGWFDP

SEQ ID NO: 35 - protein, 11 aa, synthetic construct
  REGION 1..11: note = Synthetic Sequence_1923Ab6 VL
RASQGISNYLA

SEQ ID NO: 36 - protein, 7 aa, synthetic construct
  REGION 1..7: note = Synthetic Sequence_1923Ab6 VL
AASSLQS

SEQ ID NO: 37 - protein, 9 aa, synthetic construct
  REGION 1..9: note = Synthetic Sequence_1923Ab6 VL
QQYNSYPLT

SEQ ID NO: 38 - protein, 711 aa, synthetic construct
  REGION 1..711: note = Synthetic Sequence_1923Ab7 and 1923Ab8 Heavy Chain
QVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVL

SEQ ID NO: 39 - protein, 367 aa, synthetic construct
  REGION 1..367: note = Synthetic Sequence_1923Ab7 and 1923Ab8 Heavy Chain
EIVMAQSPATLSLSPGERATLSCRASQSVSSSYLSWFQQKPGQAPRLLIYGASTRATGIPARFGGSGSGTDFTLTISSLQPEDFAVYYCQQDYNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 40 - protein, 215 aa, synthetic construct
  REGION 1..215: note = Synthetic Sequence_1923Ab8 and 1923Ab11, 1923Ab13, 1923Ab18,1923Ab20 Light Chain
EIVMAQSPATLSLSPGERATLSCRASQSVSSSYLSWFQQKPGQAPRLLIYGASTRATGIPARFGGSGSGTDFTLTISSLQPEDFAVYYCQQDYNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 711 aa, synthetic construct
  REGION 1..711: note = Synthetic Sequence_1923Ab9 Heavy Chain 1
QVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVL

SEQ ID NO: 42 - protein, 449 aa, synthetic construct
  REGION 1..449: note = Synthetic Sequence_1923Ab9 and 1923Ab10 Heavy Chain 2
QVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 711 aa, synthetic construct
  REGION 1..711: note = Synthetic Sequence_1923Ab10 Heavy Chain 1
QVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVLGGGGSGGGGSGGGGSQVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSS

SEQ ID NO: 44 - protein, 711 aa, synthetic construct
  REGION 1..711: note = Synthetic Sequence_1923Ab11 Heavy Chain
QVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVLGGGGSGGGGSGGGGSQVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSS

SEQ ID NO: 45 - protein, 449 aa, synthetic construct
  REGION 1..449: note = Synthetic Sequence_1923Ab12 Heavy Chain
QVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 484 aa, synthetic construct
  REGION 1..484: note = Synthetic Sequence_1923Ab12 Light Chain
QVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVLGGGGSGGGGSGGGGSGGGGSEIVMAQSPATLSLSPGERATLSCRASQSVSSSYLSWFQQKPGQAPRLLIYGASTRATGIPARFGGSGSGTDFTLTISSLQPEDFAVYYCQQDYNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 718 aa, synthetic construct
  REGION 1..718: note = Synthetic Sequence_1923Ab13 Heavy Chain
QVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVLGGGGSGGGGSGGGGSGGGGSQVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 712 aa, synthetic construct
  REGION 1..712: note = Synthetic Sequence_1923Ab16Heavy Chain
QVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSEIVMAQSPATLSLSPGERATLSCRASQSVSSSYLSWFQQKPGQAPRLLIYGASTRATGIPARFGGSGSGTDFTLTISSLQPEDFAVYYCQQDYNLPYTFGQGTKLEIKGGGGSGGGGSGGGGSQVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSS

SEQ ID NO: 49 - protein, 365 aa, synthetic construct
  REGION 1..365: note = Synthetic Sequence_1923Ab16 Light Chain
SYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 50 - protein, 716 aa, synthetic construct
  REGION 1..716: note = Synthetic Sequence_1923Ab17 and 1923Ab18 Heavy Chain
QVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 51 - protein, 599 aa, synthetic construct
  REGION 1..599: note = Synthetic Sequence_1923Ab19 and 1923Ab20 Heavy Chain
QVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 52 - protein, 484 aa, synthetic construct
  REGION 1..484: note = Synthetic Sequence_1923Ab19 Light Chain
EIVMAQSPATLSLSPGERATLSCRASQSVSSSYLSWFQQKPGQAPRLLIYGASTRATGIPARFGGSGSGTDFTLTISSLQPEDFAVYYCQQDYNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 53 - protein, 249 aa, synthetic construct
  REGION 1..249: note = Synthetic Sequence_scFv that binds CD137
QVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVL

SEQ ID NO: 54 - protein, 248 aa, synthetic construct
  REGION 1..248: note = Synthetic Sequence_scFv (LH) that binds CD137
YELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVLGGGGSGGGGSGGGGSQVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSS

SEQ ID NO: 55 - protein, 254 aa, synthetic construct
  REGION 1..254: note = Synthetic Sequence_scFv* with 4x (G4S) linker that binds CD137
QVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 56 - protein, 249 aa, synthetic construct
  REGION 1..249: note = Synthetic Sequence_scFv* with 3x (G4S) linker that binds CD137
QVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 57 - protein, 242 aa, synthetic construct
  REGION 1..242: note = Synthetic Sequence_scFv (LH) that binds PD-L1
EIVMAQSPATLSLSPGERATLSCRASQSVSSSYLSWFQQKPGQAPRLLIYGASTRATGIPARFGGSGSGTDFTLTISSLQPEDFAVYYCQQDYNLPYTFGQGTKLEIKGGGGSGGGGSGGGGSQVQLVQSGAEVKKPGASVKVSCKASGYTFTSYDINWVRQATGQGLEWMGWMNPNSGITGYAQRFQGRVTMTRDTSISTAYMELSSLRSEDTAVYYCARHRWGKGYLDLWGRGTLVTVSS

SEQ ID NO: 58 - protein, 15 aa, synthetic construct
  REGION 1..15: note = Synthetic Sequence_3x (G4S) linker
GGGGSGGGGSGGGGS

SEQ ID NO: 59 - protein, 20 aa, synthetic construct
  REGION 1..20: note = Synthetic Sequence_4x (G4S) linker
GGGGSGGGGSGGGGSGGGGS

SEQ ID NO: 60 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPK

SEQ ID NO: 61 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPK

SEQ ID NO: 62 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 63 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 107 aa, Homo sapiens [sequence omitted]

SEQ ID NO: 66 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 67 - protein, 137 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 68 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 69 - protein, 290 aa, synthetic construct
  REGION 1..290: note = Synthetic Sequence_Cyno PD-L1
MRIFAVFIFTIYWHLLNAFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTHLVILGAIFLLLGVALTFIFYLRKGRMMDMKKCGIRVTNSKKQRDTQLEET

SEQ ID NO: 70 - protein, 290 aa, synthetic construct
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 71 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 72 - protein, 254 aa, synthetic construct
  REGION 1..254: note = Synthetic Sequence_Cyno CD137
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 73 - protein, 255 aa, synthetic construct; identical to SEQ ID NO: 71

SEQ ID NO: 74 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 75 - protein, 455 aa, synthetic construct
  REGION 1..455: note = Synthetic Sequence_Heavy Chain that binds CD137 (Ab4 VH-hlgG1LALA)
QVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 76 - protein, 213 aa, synthetic construct
  REGION 1..213: note = Synthetic Sequence_Light Chain that binds CD137 (Ab4 VL-Lambda)
SYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 77 - protein, 5 aa, synthetic construct
NGTGV

SEQ ID NO: 78 - protein, 6 aa, synthetic construct
KGVFRT

SEQ ID NO: 79 - protein, 26 aa, synthetic construct
CTPGFHCLGAGCSMCEQDCKQGQELT

SEQ ID NO: 80 - protein, 4 aa, synthetic construct
DQKR

SEQ ID NO: 81 - protein, 4 aa, synthetic construct
KRGI

(19) United States
(12) Patent Application Publication
(10) Pub. No.: US 2025/0002599 A1
(43) Pub. Date: Jan. 2, 2025

(54) BISPECIFIC AND TRISPECIFIC BINDING PROTEINS TO PD-L1, CD137, AND/OR TGF-BETA AND USES THEREOF

(71) Applicant: NOVAROCK BIOTHERAPEUTICS, LTD., Ewing, NJ (US)

(72) Inventors: Yi Pei, Paoli, PA (US); Haichun Huang, Fremont, CA (US); Yick Loi, East Brunswick, NJ (US); Chang Hung Chen, Germantown, MD (US); Han Li, Newtown, PA (US); Di SHEN, Piscataway, NJ (US); Ming LEI, Princeton, NJ (US)

(21) Appl. No.: 18/688,720

(22) PCT Filed: Sep. 1, 2022
(86) PCT No.: PCT/US2022/075847

§ 371 Date: Mar. 1, 2024

Related U.S. Application Data
(60) Provisional application No. 63/240,404, filed on Sep. 3, 2021.

(51) Int. Cl. C07K 16/28; A61K 39/00; A61P 35/00

(52) U.S. Cl. CPC C07K 16/2878; A61P 35/00; C07K 16/2827; A61K 2039/505; C07K 2317/31; C07K 2317/565; C07K 2317/92

(57) ABSTRACT

The disclosure provides binding proteins that bind PD-L1 and CD137 (PD-L1/CD137 bispecific), binding proteins that bind PD-L1 and TGFβ (PD-L1/TGFβ bispecific), binding proteins that bind PD-L1, TGFβ, and CD137 (PD-L1/TGFβ/CD137 trispecific), and binding proteins that bind CD137, TGFβ, and PD-L1 (CD137/TGFβ/PD-L1 trispecific). The disclosure also provides pharmaceutical compositions comprising these binding proteins, and methods of their use to treat and/or prevent cancer.

### CROSS REFERENCE TO RELATED APPLICATIONS

This international patent application claims the benefit of U.S. Provisional Patent Application No. 63/240,404, filed on Sep. 3, 2021, the entire contents of which is incorporated by reference herein.

### STATEMENT REGARDING SEQUENCE LISTING

The Sequence Listing associated with this application is provided in XML format in lieu of a paper copy, and is hereby incorporated by reference into the specification. The name of the XLM file containing the Sequence Listing is 122863-5008_Sequence_Listing_ST.26.xml. The text file is about 108000 bytes, was created on or about Aug. 28, 2022, and is being submitted electronically via EFS-Web.

### FIELD

The present disclosure is in the field of immunotherapy and relates to binding proteins which bind to PD-L1 and CD137 (PD-L1/CD137 bispecific), binding proteins that bind PD-L1 and TGFβ (PD-L1/TGFβ bispecific), and binding proteins that bind PD-L1, TGFβ, and CD137 (PD-L1/TGFβ/CD137 trispecific). The present disclosure also relates to binding proteins which bind to CD137, TGFβ, and PD-L1 (CD137/TGFβ/PD-L1 trispecific). The present disclosure is also directed to polynucleotide sequences encoding these binding proteins and to cells producing them. The disclosure further relates to pharmaceutical compositions comprising these binding proteins, and to methods of their use to modulate the PD-1/PD-L1 axis and/or CD137 axis for immunotherapy.

### BACKGROUND

Immune checkpoints refer to the set of inhibitory pathways that immune cells possess in order to regulate and control the durability of the immune response while maintaining self-tolerance. Up-regulation of immune checkpoint molecules (e.g., PD-1, PD-L1, CTLA-4, TIM-3, Lag-3, VISTA, B7-H3, TIGIT, CD73, LAIR1) in the tumor microenvironment is recognized as an important mechanism that limits the anti-tumor activity of effector T cells.

Programmed cell death-1 (PD-1) is one of the predominant immune T cell receptor checkpoint receptors targeted for cancer immunotherapy. PD-1 and its ligands programmed death-ligand-1 and programmed death-ligand-2 (PD-L1 and PD-L2, respectively) act as co-inhibitory factors that regulate the balance between T cell activation, tolerance, and immunopathology. In the absence of cancer, the PD-1/PD-L1 axis functions to prevent excessive inflammation in normal tissues and helps to maintain immune tolerance to self antigens. In the presence of cancer, the PD-1 pathway is subverted to provide a main mechanism of tumor immune resistance in both tumors and peripheral tissue. The axis is repurposed to promote cancer development and progression by enhancing tumor cell survival.

The PD-1 ligands, PD-L1 (also known as cluster of differentiation 274 (CD274)) or B7 homolog 1 (B7-H1), and PD-L2 (also known as B7-DC or CD273)) are normally expressed on the surface of dendritic cells or macrophages. PD-L1 is also overexpressed on tumor cells or on non-transformed cells in the tumor micro-environment (TME). The interaction of PD-1 with PD-L1 leads to the inhibition of T cell receptor (TCR) signaling and CD28 co-stimulation and ultimately leads to T cell inactivation and loss of proliferative capacity. PD-L1 expression in the tumor microenvironment allows cancer cells to exploit the PD-1/PD-L1 checkpoint pathway as an evasion mechanism to prevent or avoid a patient's antigen-specific T cell immunologic response.

The blockade of the PD-1 receptor or its ligand with antibodies deprives the cancer cells of their evasion strategy and enhances or promotes antitumor immune responses. To date, six PD-1/PD-L1 immune checkpoint inhibitors (ICIs) have received U.S. Food and Drug Administration (FDA) approval: three PD-1 inhibitors (nivolumab, pembrolizumab, and cemiplimab), and three PD-L1 inhibitors (atezolizumab, durvalumab, and avelumab).

Cancer immunotherapy with therapeutic monoclonal antibodies to negate the PD-1/PD-L1 checkpoint inhibition of immune responses has revolutionized the treatment of a wide variety of malignancies. However, some initial responders eventually develop acquired resistance to monotherapy and experience relapsed disease and a significant number of patients demonstrate primary resistance and fail to respond to PD-1/PD-L1 immune checkpoint blockade. In light of the reported prevalence of checkpoint inhibitor therapy resistance, there is an unmet need for additional therapeutics for refractory or relapsing cancer patients.

CD137 (4-1BB or TNFRSF9) is a member of the TNF Receptor Superfamily. CD137 expresses on both innate and adaptive immune cells. It plays a multifaceted role in the tumor microenvironment (TME). It is prevalently upregulated on T cells in TME and provides co-stimulation to CD8 and CD4 T cell activation, proliferation, and survival. It also modulates other cell functions in TME, such as promoting NK cells to interact with CD8 T cells, enhancing tumor antigen presentation on DC cells. A CD137 agonist can enhance the anti-tumor immune response and may work synergistically with other anti-tumor agents, including PD1/PD-L1 blocking antibodies.

Secretion of TGFβ is another leading contributor to immune evasion and tumor progression. TGFβ promotes tumor progression and immune-suppressing by preventing T cell proliferation and decrease the effector function of both T and NK cells. It also enhances the function of T regulatory cells and induces epithelial-to-mesenchymal transition (EMT).

Since PD1, CD137, and TGFβ regulate independent immune suppression or immune stimulative pathway, it is possible that targeting PD1 and CD137, PD1 and TGFβ, or targeting PD1, CD137 and TGFβ will increase the anti-tumor efficacy, especially for immune-excluded and immune-desert tumors.

### SUMMARY

The present disclosure addresses the above need by providing multispecific binding proteins including, for example, binding proteins that bind PD-L1 and CD137 (PD-L1/CD137 bispecific), binding proteins that bind PD-L1 and TGFβ (PD-L1/TGFβ bispecific), and binding proteins that bind PD-L1, TGFβ, and CD137 (PD-L1/TGFβ/CD137 trispecific). In an exemplary embodiment, a PD-L1/CD137 bispecific is a binding protein that binds PD-L1 and CD137 and comprises: (a) an antibody scaffold module in an IgG format (e.g., a Y-shaped symmetrical or asymmetrical antibody comprising two heavy chains and two light chains) comprising a first antigen-binding site that binds PD-L1 and a second antigen-binding site that binds PD-L1; (b) at least one first binding module comprising a third antigen-binding site that binds CD137. In an exemplary embodiment, a PD-L1/TGFβ bispecific is a binding protein that binds PD-L1 and TGFβ and comprises: (a) an antibody scaffold module in an IgG format comprising a first antigen-binding site that binds PD-L1 and a second antigen-binding site that binds PD-L1; (b) at least one first binding module comprising a third antigen-binding site that binds TGFβ. In an exemplary embodiment, a PD-L1/TGFβ/CD137 trispecific is a binding protein that comprises (a) an antibody scaffold module in an IgG format comprising a first antigen-binding site that binds PD-L1 and a second antigen-binding site that binds PD-L1; (b) at least one first binding module comprising a third antigen-binding site that binds TGFβ; and (c) at least one second binding module comprising a fourth antigen-binding site that binds CD137.

The present disclosure also relates to binding proteins which bind human CD137, including binding proteins that bind CD137, TGFβ, and PD-L1 (CD137/TGFβ/PD-L1 trispecific). More specifically, the disclosure relates to binding proteins that bind CD137 and to their use to stimulate the CD137 and to promote durable anti-tumor immune responses. In an exemplary embodiment, a binding protein may bind CD137 and comprises an antibody scaffold module in an IgG format comprising a first antigen-binding site that binds CD137 and a second antigen-binding site that binds CD137. In an exemplary embodiment, a CD137/TGFβ/PD-L1 trispecific is a binding protein that comprises (a) an antibody scaffold module in an IgG format comprising a first antigen-binding site that binds CD137 and a second antigen-binding site that binds CD137; (b) at least one first binding module comprising a third antigen-binding site that binds TGFβ; and (c) at least one second binding module comprising a fourth antigen-binding site that binds PD-L1.

Despite the advances in immunotherapy attributed to the use of PD-1/PD-L1 blockers there is a need for binding proteins that can bind both PD-L1 and to a co-stimulatory target on T cells and/or reverse immunosuppression in the tumor microenvironment by neutralizing TGFβ. The three TGFβ isoforms, TGFβ1, TGFβ2 and TGFβ3 are highly expressed in many tumors, which promotes cancer progression primarily by suppressing both the innate and adaptive immune systems. And their serum concentrations correlate with poor prognosis. In the tumor microenvironment, TGFβ promotes tumor progression by stromal modification, angiogenesis, and induction of epithelial-mesenchymal transition (EMT). TGFβ signaling induces the differentiation of Treg cells and drives metastasis through myeloid cells. Moreover, TGFβ1 can directly inhibit T cell and NK cell function.

A binding protein can simultaneously bind two or three epitopes of a single antigen or to more than one target antigen, allowing for multiple mechanistic functions and potential synergistic effects that cannot be achieved by a monospecific therapeutic antibody or fusion protein. Advantageously the use of a binding protein that binds two or three antigens may have a lower risk of toxicity than the risk associated with the use of multiple therapeutic agents.

Broadly speaking, the disclosed bispecifics and trispecifics disclosed herein are based on natural IgG scaffolds with or without modifications to promote heavy chain heterodimerization and are characterized by two or three binding specificities (e.g., PD-L1, CD137 and/or TGFβ) contributed by antigen binding sites derived from antibody Fabs or scFv fragments and/or a fusion protein prepared from the extracellular domain (ECD) of TGFβRII receptor appended to the N-terminus or C-terminus of an IgG heavy or light chain in the IgG scaffold.

In some embodiments, the PD-L1/CD137 bispecifics, PD-L1/TGFβ bispecifics or PD-L1/TGFβ/CD137 trispecifics exhibit one or more of the following structural characteristics, alone or in combination:
(a) have anti-CD137 in an scFv format,
(b) have anti-PD-L1 in an scFv format,
(c) have an scFv fused at the N-terminus of an antibody light chain in the IgG scaffold,
(d) have an scFv fused at the N-terminus of an antibody heavy chain in the IgG scaffold,
(e) have an scFv fused at the C-terminus of an antibody light chain in the IgG scaffold,
(f) have an scFv fused at the C-terminus of an antibody heavy chain in the IgG scaffold,
(g) have an scFv of CD137 existing in a monovalent or a divalent format,
(h) contain human TGFβRII fused at the C-terminus of the light chain in the IgG scaffold, or
(i) contain human TGFβRII fused at the C-terminus of the heavy chain in the IgG scaffold.

In some embodiments, the PD-L1/CD137 bispecifics, PD-L1/TGFβ bispecifics, and PD-L1/TGFβ/CD137 trispecifics provide an immunotherapy that targets exhausted PD-1⁺ CD8 T cells and reinvigorates dysfunctional tumor infiltrating lymphocytes (TILs). Such binding proteins that bind PD-L1 may be particularly beneficial as therapeutics in solid tumors characterized by microenvironments enriched in exhausted TCD8⁺ cells and/or regulatory T cells that contribute to PD-1/PD-L1 resistance. In practice, blocking the PD-1/PD-L1 signaling axis reduces immunosuppressive signals present in the TME and enhances anti-tumor immunity, which in turn produces durable clinical responses that prolong patient survival.

In some embodiments, the disclosed binding proteins that bind PD-L1 are bispecific, tetravalent molecules specific for PD-L1 and either CD137 or human TGFβ.

In other embodiments, the disclosed binding proteins that bind PD-L1 are molecules specific for PD-L1 and both CD137 and human TGFβ.

In some embodiments, the disclosed CD137/TGFβ/PD-L1 trispecifics are designed to provide an immunotherapy that targets CD137 expressing immune cells in the tumor microenvironment. The binding proteins that bind CD137 may be particularly beneficial as therapeutics in solid tumors. In practice, activating the CD137 signaling axis will enhance the T effector function present in the TME and enhance anti-tumor immunity, which could in turn produce durable clinical responses that prolong patient survival.

In other embodiments, the disclosed binding proteins bind CD137 and both PD-L1 and human TGFβ.

According to some embodiments, the disclosed binding proteins that bind PD-L1 comprise a set of six complementarity determining region (CDR) sequences selected from the group consisting of three CDRs of an anti-PD-L1 antibody heavy chain (HC) variable region sequence selected from SEQ ID NOS: 1 and 3 and three CDRs of a light chain variable region sequence selected from SEQ ID NOS: 2 and 4.

In some embodiments, the binding proteins that bind PD-L1 comprise a heavy chain variable region sequence comprising CDR1: SEQ ID NO: 5, CDR2: SEQ ID NO: 6, and CDR3: SEQ ID NO: 7; and/or a light chain variable region sequence comprising CDR1: SEQ ID NO: 8, CDR2: SEQ ID NO: 9, and CDR3: SEQ ID NO: 10.

In some embodiments, the binding proteins that bind PD-L1 comprise a heavy chain variable region sequence comprising CDR: SEQ ID NO: 11, CDR2: SEQ ID NO: 12, and CDR3: SEQ ID NO: 13; and/or a light chain variable region sequence comprising CDR1: SEQ ID NO: 14, CDR2: SEQ ID NO: 9, and CDR3: SEQ ID NO: 15.

In some embodiments, the binding proteins that bind PD-L1 comprise a heavy chain variable region sequence set forth in SEQ ID NO: 1 or SEQ ID NO: 3, or an analogue or derivative thereof having at least 90% sequence identity to SEQ ID NO: 1 or SEQ ID NO: 3.

In other embodiments, the binding proteins that bind PD-L1 comprise a light chain variable region sequence set forth in SEQ ID NOs: 2 or SEQ ID NO: 4 or an analogue or derivative thereof having at least 90% sequence identity to SEQ ID NO: 2 or SEQ ID NO: 4.

In other embodiments, the binding proteins that bind PD-L1 comprise a heavy chain variable region sequence set forth in SEQ ID NOs: 1 or SEQ ID NO: 3 and a light chain variable region sequence set forth in SEQ ID NO: 2 or SEQ ID NO: 4.

In some embodiments, the binding proteins that bind PD-L1 comprise a heavy chain variable region sequence and a light chain variable region sequence, selected from the following combinations:
(a) a heavy chain variable region sequence comprising SEQ ID NO: 1 and a light chain variable region sequence comprising SEQ ID NO: 2; or
(b) a heavy chain variable region sequence comprising SEQ ID NO: 3 and a light chain variable region sequence comprising SEQ ID NO: 4.

In some embodiments, a binding protein that binds PD-L1 comprises:
(a) a heavy chain variable region sequence comprising CDR1: SEQ ID NO: 5, CDR2: SEQ ID NO: 6, and CDR3: SEQ ID NO: 7; and/or a light chain variable region sequence comprising CDR1: SEQ ID NO: 8, CDR2: SEQ ID NO: 9, and CDR3: SEQ ID NO: 10; or
(b) a heavy chain variable region sequence comprising CDR1: SEQ ID NO: 11, CDR2: SEQ ID NO: 12, and CDR3: SEQ ID NO: 13; and/or a light chain variable region sequence comprising CDR1: SEQ ID NO: 14, CDR2: SEQ ID NO: 9, and CDR3: SEQ ID NO: 15.

In some embodiments, a binding protein that binds PD-L1 is provided wherein the first and second antigen-binding sites bind PD-L1, and wherein the antibody scaffold module comprises:
(i) a heavy chain variable region sequence as set forth in SEQ ID NO: 1, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 2, and a light chain constant region sequence as set forth in SEQ ID NO: 65 or SEQ ID NO: 66; or
(ii) a heavy chain variable region sequence as set forth in SEQ ID NO: 3, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 4, and a light chain constant region sequence as set forth in SEQ ID NO: 65 or SEQ ID NO: 66.

In some embodiments, a binding protein that binds PD-L1 is provided wherein the first and second antigen-binding sites bind PD-L1, and wherein the antibody scaffold module comprises:
a heavy chain sequence as set forth in SEQ ID NO: 45 and a light chain sequence as set forth in SEQ ID NO: 40.

In some embodiments, the binding proteins that bind PD-L1 comprise one or more heavy chain variable region CDRs disclosed in Table 1 and/or one or more light chain variable region CDRs disclosed in Table 2.

In some embodiments, the binding proteins that bind PD-L1 exhibit one or more of the following structural and functional characteristics, alone or in combination:
(a) is specific for human PD-L1,
(b) cross-reacts with cynomolgus PD-L1,
(c) disrupts the interaction of PD-1 and PD-L1, or
(d) dis-inhibits PD-1/PD-L1 checkpoint-mediated inhibition of T cells.

In some embodiments, the binding proteins that bind PD-L1 specifically bind to human cells expressing endogenous levels of PD-L1 and to host cells engineered to overexpress human PD-L1. The binding proteins that bind PD-L1 may also bind to cells overexpressing human or cyno PD-L1 with subnanomolar EC₅₀ values.

In some embodiments, the binding proteins that bind PD-L1 cross-react with cynomolgus monkey PD-L1 (cynoPD-L1) and do not demonstrate cross-reactive binding to mouse PD-L1 (mu-PD-L1).

In some embodiments, the binding proteins that bind PD-L1 disrupt the human PD-1/PD-L1 binding interaction.

In some embodiments, the binding proteins that bind PD-L1 dis-inhibits PD-1/PD-L1 checkpoint-mediated inhibition of T cells.

In some embodiments, the binding proteins that bind PD-L1 further comprise an Fc region that is engineered to abolish/minimize cross-linking activity with FcγRs, which silence or eliminate Fc-mediated effector functions of T cells.

The present disclosure also provides isolated polynucleotide sequences encoding at least one of the above binding proteins that bind PD-L1.

The present disclosure also provides vectors comprising at least one of the above polynucleotide sequences.

The present disclosure also provides cells comprising one of the above polynucleotide sequences, or one of the above vectors.

The present disclosure also provides pharmaceutical compositions comprising or consisting of at least one of the binding proteins that bind PD-L1, and optionally a pharmaceutically acceptable diluent, carrier, vehicle and/or excipient. Such a pharmaceutical composition may be used for the treatment of cancer.

The present disclosure also relates to methods for treatment of cancer in a patient comprising administering to the patient a therapeutically effective amount of at least one of the disclosed binding proteins that bind PD-L1, alone or in combination with another therapeutic agent.

According to some embodiments, the binding proteins that bind CD137 comprise a set of six complementarity determining region (CDR) sequences selected from the group consisting of three CDRs of an anti-CD137 antibody heavy chain (HC) variable region sequence selected from SEQ ID NOS: 16, 18, and 20 and three CDRs of a light chain variable region sequence selected from SEQ ID NOS: 17, 19, and 21.

In some embodiments, the binding proteins that bind CD137 comprise a heavy chain variable region sequence comprising CDR1: SEQ ID NO: 5, CDR2: SEQ ID NO: 22, and CDR3: SEQ ID NO: 23; and/or a light chain variable region sequence comprising CDR1: SEQ ID NO: 24, CDR2: SEQ ID NO: 25, and CDR3: SEQ ID NO: 26.

In some embodiments, the binding proteins that bind CD137 comprise a heavy chain variable region sequence comprising CDR1: SEQ ID NO: 27, CDR2: SEQ ID NO: 28, and CDR3: SEQ ID NO: 29; and/or a light chain variable region sequence comprising CDR1: SEQ ID NO: 30, CDR2: SEQ ID NO: 9, and CDR3: SEQ ID NO: 31.

In some embodiments, the binding proteins that bind CD137 comprise a heavy chain variable region sequence comprising CDR1: SEQ ID NO: 32, CDR2: SEQ ID NO: 33, and CDR3: SEQ ID NO: 34; and/or a light chain variable region sequence comprising CDR1: SEQ ID NO: 35, CDR2: SEQ ID NO: 36, and CDR3: SEQ ID NO: 37.

In some embodiments, the binding proteins that bind CD137 comprise a heavy chain variable region sequence as set forth in SEQ ID NOs: 16, 18, or 20, or an analogue or derivative thereof having at least 90% sequence identity to SEQ ID NOs: 16, 18, or 20.

In other embodiments, the binding proteins that bind CD137 comprise alight chain variable region sequence as set forth in SEQ ID NOs: 17, 19, or 21, or an analogue or derivative thereof having at least 90% sequence identity to SEQ ID NOs: 17, 19, or 21.

In other embodiments, the binding proteins that bind CD137 comprise a heavy chain variable region sequence as set forth in SEQ ID NOs: 16, 18 or 20 and a light chain variable region sequence as set forth in SEQ ID NOs: 17, 19 or 21.

In some embodiments, the binding proteins that bind comprise a heavy chain variable region sequence and a light chain variable region sequence, selected from the following combinations:
(a) a heavy chain variable region sequence comprising SEQ ID NO: 16 and a light chain variable region sequence comprising SEQ ID NO: 17;
(b) a heavy chain variable region sequence comprising SEQ ID NO: 18 and a light chain variable region sequence comprising SEQ ID NO: 19; and
(c) a heavy chain variable region sequence comprising SEQ ID NO: 20 and a light chain variable region sequence comprising SEQ ID NO: 21.

In some embodiments, a binding protein that binds CD137 is provided, comprising:
(a) a heavy chain variable region sequence comprising CDR1: SEQ ID NO: 5, CDR2: SEQ ID NO: 22, and CDR3: SEQ ID NO: 23; and/or a light chain variable region sequence comprising CDR1: SEQ ID NO: 24, CDR2: SEQ ID NO: 25, and CDR3: SEQ ID NO: 26,
(b) a heavy chain variable region sequence comprising CDR1: SEQ ID NO: 27, CDR2: SEQ ID NO: 28, and CDR3: SEQ ID NO: 29, and/or a light chain variable region sequence comprising CDR1: SEQ ID NO: 30, CDR2: SEQ ID NO: 9, and CDR3: SEQ ID NO: 31; or
(c) a heavy chain variable region sequence comprising CDR1: SEQ ID NO: 32, CDR2: SEQ ID NO: 33, and CDR3: SEQ ID NO: 34; and/or a light chain variable region sequence comprising CDR1: SEQ ID NO: 35, CDR2: SEQ ID NO: 36, and CDR3: SEQ ID NO: 37.

In some embodiments, a binding protein that binds CD137 is provided wherein the first and second antigen-binding sites bind CD137, and wherein the antibody scaffold module comprises:
(i) a heavy chain variable region sequence as set forth in SEQ ID NO: 16, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 17, and a light chain constant region sequence as set forth in SEQ ID NO: 65 or SEQ ID NO: 66;
(ii) a heavy chain variable region sequence as set forth in SEQ ID NO: 18, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 19, and a light chain constant region sequence as set forth in SEQ ID NO: 65 or SEQ ID NO: 66; or
(iii) a heavy chain variable region sequence as set forth in SEQ ID NO: 20, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 21, and a light chain constant region sequence as set forth in SEQ ID NO: 65 or SEQ ID NO: 66.

In some embodiments, a binding protein that binds CD137 is provided wherein the first and second antigen-binding sites bind CD137, and wherein the antibody scaffold module comprises a heavy chain sequence as set forth in SEQ ID NO: 75 and a light chain sequence as set forth in SEQ ID NO: 76.

In some embodiments, the anti-CD137 antibodies comprise one or more heavy chain variable region CDRs disclosed in Table 3 and/or one or more light chain variable region CDRs disclosed in Table 4.

In some embodiments, the binding proteins that bind CD137 exhibit one or more of the following structural and functional characteristics, alone or in combination:
(a) is specific for human CD137,
(b) cross-reacts with cynomolgus CD137,
(c) disrupts (e.g., reduces or prevents) human CD137L binding to CD137,
(d) exhibits fast on and fast off properties to CD137;
(e) possess crosslinking dependent agonistic activity for CD137 signaling, or
(f) activates T cells in crosslinking dependent manner.

In some embodiments, the binding proteins that bind CD137 specifically bind to human cells expressing endogenous levels of CD137 and to host cells engineered to overexpress human CD137. In some embodiments, the binding proteins that bind CD137 bind to cells overexpressing human or cyno CD137 with EC₅₀ values ranging from 0.2 to 1.1 nM (e.g., 0.2 nM, 0.3 nM, 0.4 nM, 0.5 nM, 0.6 nM, 0.7 nM, 0.8 nM, 0.9 nM, 1.0 nM or 1.1 nM.

In some embodiments, the binding proteins that bind to CD137 have a fast on and fast off kinetic property.

In some embodiments, the binding proteins that bind CD137 cross-react with cynomolgus monkey CD137 (cynoCD137) and do not demonstrate cross-reactive binding to mouse CD137 (mu-CD137).

In some embodiments, the binding proteins that bind CD137 disrupt the CD137 ligand/CD137 binding interaction.

In some embodiments, the binding proteins that bind CD137 possess crosslinking dependent agonistic activity for CD137 signaling.

In some embodiments, the binding proteins that bind CD137 activate T cells in crosslinking dependent manner.

In some embodiments, the binding proteins that bind CD137 further comprise an Fc region that is engineered to abolish/minimize cross-linking activity with FcγRs, which silence or eliminate Fc-mediated effector functions of T cells.

The present disclosure also provides isolated polynucleotide sequences encoding at least one of the above binding proteins that bind CD137.

The present disclosure also provides vectors comprising at least one of the above polynucleotide sequences.

The present disclosure also provides cells comprising one of the above polynucleotide sequences, or one of the above vectors.

The present disclosure also provides pharmaceutical compositions comprising or consisting of at least one of the binding proteins that bind CD137, and optionally a pharmaceutically acceptable diluent, carrier, vehicle and/or excipient. Such a pharmaceutical composition may be used for the treatment of cancer.

The present disclosure also relates to methods for treatment of cancer in a patient comprising administering to the patient a therapeutically effective amount of at least one of the disclosed binding proteins that bind CD137, alone or in combination with another therapeutic agent.

In an exemplary embodiment, a PD-L1/CD137 bispecific is a binding protein that binds PD-L1 and CD137 and comprises: (a) an antibody scaffold module in an IgG format comprising a first antigen-binding site that binds PD-L1 and a second antigen-binding site that binds PD-L1; (b) at least one first binding module comprising a third antigen-binding site that binds CD137.

In an exemplary embodiment, the first antigen-binding site and the second antigen-binding site of the PD-L1/CD137 bispecific comprise a heavy chain variable region sequence comprising CDR1: SEQ ID NO: 5, CDR2: SEQ ID NO: 6, and CDR3: SEQ ID NO: 7; and a light chain variable region sequence comprising CDR1: SEQ ID NO: 8, CDR2: SEQ ID NO: 9, and CDR3: SEQ ID NO: 10. In an exemplary embodiment, the first antigen-binding site and the second antigen-binding site of the PD-L1/CD137 bispecific comprise a heavy chain variable region sequence comprising CDR1: SEQ ID NO: 11, CDR2: SEQ ID NO: 12, and CDR3: SEQ ID NO: 13; and a light chain variable region sequence comprising CDR1: SEQ ID NO: 14, CDR2: SEQ ID NO: 9, and CDR3: SEQ ID NO: 15.

In an exemplary embodiment, the antibody scaffold module of the PD-L1/CD137 bispecific comprises a heavy chain variable region sequence as set forth in SEQ ID NO: 1 or SEQ ID NO: 3; and a light chain variable region sequence as set forth in SEQ ID NO: 2 or SEQ ID NO: 4.

In an exemplary embodiment, the antibody scaffold module of the PD-L1/CD137 bispecific comprises, a heavy chain variable region sequence as set forth in SEQ ID NO: 1 and a light chain variable region sequence as set forth in SEQ ID NO: 2. In an exemplary embodiment, the antibody scaffold module of the PD-L1/CD137 bispecific comprises, a heavy chain variable region sequence as set forth in SEQ ID NO: 3 and a light chain variable region sequence as set forth in SEQ ID NO: 4.

In an exemplary embodiment, the antibody scaffold module of the PD-L1/CD137 bispecific comprises, a heavy chain variable region sequence as set forth in SEQ ID NO: 1, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 2, and a light chain constant region sequence as set forth in SEQ ID NO: 65 or SEQ ID NO: 66. In an exemplary embodiment, the antibody scaffold module of the PD-L1/CD137 bispecific comprises a heavy chain variable region sequence as set forth in SEQ ID NO: 3, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 4, and a light chain constant region sequence as set forth in SEQ ID NO: 65 or SEQ ID NO: 66.

In an exemplary embodiment, the antibody scaffold module of the PD-L1/CD137 bispecific comprises a heavy chain sequence as set forth in SEQ ID NO: 42 and a light chain sequence as set forth in SEQ ID NO: 40. In an exemplary embodiment, the antibody scaffold module of the PD-L1/CD137 bispecific comprises a heavy chain sequence as set forth in SEQ ID NO: 45 and a light chain sequence as set forth in SEQ ID NO: 40.

In an exemplary embodiment, the PD-L1/CD137 bispecific has one first binding module. In an exemplary embodiment, the PD-L1/CD137 bispecific has two first binding modules. In an exemplary embodiment, the PD-L1/CD137 bispecific has an antibody scaffold module which comprises a heavy chain sequence which comprises a C-terminus and a N-terminus, and wherein this antibody scaffold module comprises a light chain sequence which comprises a C-terminus and an N-terminus, and the first binding module is covalently attached to the C-terminus of the antibody scaffold module heavy chain sequence, the C-terminus of the antibody scaffold module light chain sequence, the N-terminus of the antibody scaffold module heavy chain sequence, the N-terminus of the antibody scaffold module light chain sequence, or combinations thereof, optionally wherein the first binding module and the antibody scaffold module are covalently attached to each other directly or through an interlinker. In an exemplary embodiment, the first binding module and the antibody scaffold module of the PD-L1/CD137 bispecific are covalently attached to each other through an interlinker, and the interlinker is the sequence, from N- to C-terminus, as set forth in SEQ ID NO: 58. In an exemplary embodiment, the first binding module and the antibody scaffold module of the PD-L1/CD137 bispecific are covalently attached to each other through an interlinker, and the interlinker is the sequence, from N- to C-terminus, as set forth in SEQ ID NO: 59. In an exemplary embodiment, the first binding module of the PD-L1/CD137 bispecific is covalently attached to the C-terminus of the antibody scaffold module heavy chain sequence. In an exemplary embodiment, the first binding module of the PD-L1/CD137 bispecific is covalently attached to the C-terminus of the antibody scaffold module light chain sequence. In an exemplary embodiment, when there is more than one first binding module in the PD-L1/CD137 bispecific, each is covalently attached to a different antibody scaffold module sequence or to a different end of the antibody scaffold module.

In an exemplary embodiment, the first binding module in the PD-L1/CD137 bispecific is a scFv, which comprises a heavy chain variable region sequence and a light chain variable sequence, wherein the sequences are covalently attached to each other directly or through a scFv fusion linker. In an exemplary embodiment, the scFv fusion linker comprises glycine and serine. In an exemplary embodiment, the scFv fusion linker comprises the sequence Gly-Gly-Gly-Ser. In an exemplary embodiment, the scFv fusion linker comprises the sequence set forth in SEQ ID NO: 58. In an exemplary embodiment, the scFv fusion linker is the sequence set forth in SEQ ID NO: 58. In an exemplary embodiment, the scFv fusion linker comprises the sequence set forth in SEQ ID NO: 59. In an exemplary embodiment, the scFv fusion linker is the sequence set forth in SEQ ID NO: 59. In an exemplary embodiment, the first binding module in the PD-L1/CD137 bispecific comprises a sequence as set forth in SEQ ID NO: 53. In an exemplary embodiment, the first binding module in the PD-L1/CD137 bispecific comprises a sequence, as set forth in SEQ ID NO: 54. In an exemplary embodiment, the first binding module in the PD-L1/CD137 bispecific comprises a sequence as set forth in SEQ ID NO: 55. In an exemplary embodiment, the first binding module in the PD-L1/CD137 bispecific comprises a sequence as set forth in SEQ ID NO: 56.

In an exemplary embodiment, the first binding module in the PD-L1/CD137 bispecific comprises, from N- to C-terminus: a heavy chain variable region sequence comprising CDR1: SEQ ID NO: 5, CDR2: SEQ ID NO: 22, and CDR3: SEQ ID NO: 23; and a light chain variable region sequence comprising CDR1: SEQ ID NO: 24, CDR2: SEQ ID NO: 25, and CDR3: SEQ ID NO: 26. In an exemplary embodiment, the first binding module in the PD-L1/CD137 bispecific comprises, from N- to C-terminus: a heavy chain variable region sequence comprising CDR1: SEQ ID NO: 27, CDR2: SEQ ID NO: 28, and CDR3: SEQ ID NO: 29; and a light chain variable region sequence comprising CDR1: SEQ ID NO: 30, CDR2: SEQ ID NO: 9, and CDR3: SEQ ID NO: 31. In an exemplary embodiment, the first binding module in the PD-L1/CD137 bispecific comprises, from N- to C-terminus: a heavy chain variable region sequence comprising CDR1: SEQ ID NO: 32, CDR2: SEQ ID NO: 33, and CDR3: SEQ ID NO: 34; and a light chain variable region sequence comprising CDR1: SEQ ID NO: 35, CDR2: SEQ ID NO: 36, and CDR3: SEQ ID NO: 37.

In an exemplary embodiment, the first binding module in the PD-L1/CD137 bispecific comprises, from N- to C-terminus: a heavy chain variable region sequence as set forth in SEQ ID NO: 16 and a light chain variable region sequence as set forth in SEQ ID NO: 17. In an exemplary embodiment, the first binding module in the PD-L1/CD137 bispecific comprises, from N- to C-terminus: a heavy chain variable region sequence as set forth in SEQ ID NO: 18 and a light chain variable region sequence as set forth in SEQ ID NO: 19. In an exemplary embodiment, the first binding module in the PD-L1/CD137 bispecific comprises, from N- to C-terminus: a heavy chain variable region sequence as set forth in SEQ ID NO: 20 and a light chain variable region sequence as set forth in SEQ ID NO: 21.

In an exemplary embodiment, the PD-L1/CD137 bispecific comprises, from N- to C-terminus: the heavy chain sequence of the antibody scaffold module and the first binding module as set forth in SEQ ID NO: 38; and the light chain sequence of the antibody scaffold module as set forth in SEQ ID NO: 40. In an exemplary embodiment, the PD-L1/CD137 bispecific comprises, from N- to C-terminus: the heavy chain sequence of the antibody scaffold module and the first binding module as set forth in SEQ ID NO: 44; and the light chain sequence of the antibody scaffold module as set forth in SEQ ID NO: 40. In an exemplary embodiment, the PD-L1/CD137 bispecific comprises, from N- to C-terminus: the heavy chain sequence of the antibody scaffold module as set forth in SEQ ID NO: 45; and the light chain sequence of the antibody scaffold module and the first binding module as set forth in SEQ ID NO: 46. In an exemplary embodiment, the PD-L1/CD137 bispecific comprises, from N- to C-terminus: the heavy chain sequence of the antibody scaffold module and the first binding module as set forth in SEQ ID NO: 47; and the light chain sequence of the antibody scaffold module as set forth in SEQ ID NO: 40. In an exemplary embodiment, the PD-L1/CD137 bispecific comprises, from N- to C-terminus: the heavy chain sequence of the antibody scaffold module and the first binding module as set forth in SEQ ID NO: 50; and the light chain sequence of the antibody scaffold module as set forth in SEQ ID NO: 40.

In an exemplary embodiment, the antibody scaffold module of the PD-L1/CD137 bispecific further comprises a constant region. In an exemplary embodiment, the constant region of the antibody scaffold module of the PD-L1/CD137 bispecific comprises at least one Fc silencing mutation. In an exemplary embodiment, the Fc silencing mutation in the constant region of the antibody scaffold module of the PD-L1/CD137 bispecific is L234AL235A or N297A. In an exemplary embodiment, the constant region of the antibody scaffold module of the PD-L1/CD137 bispecific comprises knobs-in-holes (KiH) mutations.

According to some embodiments, the PD-L1/CD137 bispecifics (e.g., 1923Ab8, 1923Ab11, 1923Ab12, 1923Ab13, and 1923Ab18) are capable of effectively blocking the interactions between PD-L1 and its receptor PD-1 and between CD137 and its ligand. The disclosed PD-L1/CD137 bispecifics comprise amino acid sequences derived from one of the binding proteins that bind PD-L1 disclosed herein (e.g., 1923Ab2 or 1923Ab3) as a PD-L1 antibody scaffold module and amino acid sequences derived from one of the binding proteins that bind CD137 (e.g., 1923Ab4, 1923Ab5 or 1923Ab6) disclosed herein as a CD137 first binding module.

In some embodiments, the PD-L1/CD137 bispecific comprises a binding protein that binds PD-L1 and that disrupts the PD-1/PD-L1 binding interaction and dis-inhibits PD-1/PD-L1 checkpoint-mediated inhibition of T cells. As exemplified herein, in non-limiting examples, the antibody scaffold module that binds PD-L1 may comprise a single-chain variable fragment (e.g., a fusion protein of the variable regions of the heavy (VH) and light (VL) chains of one of the disclosed binding proteins that bind PD-L1, connected with a linker peptide) (scFv).

In some embodiments, the PD-L1/CD137 bispecific comprises a CD137-binding module comprised of a fragment derived from one of the disclosed binding proteins that bind CD137. In one embodiment, the CD137 binding module may be in the form of a binding fragment derived from one of the binding proteins that bind CD137 disclosed herein. As exemplified herein, in non-limiting examples, the CD137 binding module may comprise a single-chain variable fragment (e.g., a fusion protein of the variable regions of the heavy (VH) and light (VL) chains of one of the disclosed binding proteins that bind CD137, connected with a linker peptide). Alternatively, the CD137 binding module may be in the form of an IgG molecule.

In some embodiments, the PD-L1/CD137 bispecific 1923Ab8 comprises an antibody scaffold module having two Fabs from 1923Ab3 that bind PD-L1, a human IgG1 Fc comprising two Fc constant chains with L234A L235A mutations (SEQ ID NO: 61), and an scFv fragment (VH precedes VL) (SEQ ID NO: 53) derived from 1923Ab4 attached to the C-terminus of each of the two Fc constant chains.

In some embodiments, the PD-L1/CD137 bispecific 1923Ab11 comprises an antibody scaffold module having two Fabs from 1923Ab3 that bind PD-L1, a human IgG1 Fc comprising two Fc constant chains with L234A L235A mutations (SEQ ID NO: 61), and scFv fragments (VL precedes VH) (SEQ ID NO: 54), derived from 1923Ab4 attached to the C-terminus of each of the two Fc constant chains.

In some embodiments, the PD-L1/CD137 bispecific 1923Ab12 comprises an antibody scaffold module having two Fabs from 1923Ab3 that bind PD-L1, a human IgG1 Fc comprising two constant chains with L234A L235A mutations (SEQ ID NO: 61), and disulfide bond-stabilized scFv fragments (VH precedes VL) (SEQ ID NO: 56), derived from 1923Ab4 attached to the N-terminus of each of light chain in the Fabs.

In some embodiments, the PD-L1/CD137 bispecific 1923Ab13 comprises an antibody scaffold module having two Fabs from 1923Ab3 that bind PD-L1, a human IgG1 Fc comprising two constant chains with L234A L235A mutations (SEQ ID NO: 61), and disulfide bond-stabilized scFv fragments (VH precedes VL) (SEQ ID NO: 56), derived from 1923Ab4 attached to the N-terminus of each of the heavy chains in the Fabs.

In some embodiments, the PD-L1/CD137 bispecific 1923Ab18 comprises an antibody scaffold module having two Fabs from 1923Ab3 that bind PD-L1, a human IgG1 Fc comprising two Fc constant chains with L234A L235A mutations (SEQ ID NO: 61), and disulfide bond-stabilized scFv fragments (VH precedes VL) (SEQ ID NO: 55) derived from 1923Ab4 attached to the C-terminus of each of the Fc constant chains.

In some embodiments, the PD-L1/CD137 bispecific comprise a variable heavy chain sequence and a variable light chain sequence, selected from the following combinations:
(a) a heavy chain sequence comprising SEQ ID NO: 38 and a light chain sequence comprising SEQ ID NO: 40;
(b) a heavy chain sequence comprising SEQ ID NO: 44 and a light chain sequence comprising SEQ ID NO: 40;
(c) a heavy chain sequence comprising SEQ ID NO: 45 and a light chain sequence comprising SEQ ID NO: 46;
(d) a heavy chain sequence comprising SEQ ID NO: 47 and a light chain sequence comprising SEQ ID NO: 40; and
(e) a heavy chain sequence comprising SEQ ID NO: 50 and a light chain sequence comprising SEQ ID NO: 40.

In some embodiments, the PD-L1/CD137 bispecific comprises a heavy chain sequence selected from the group consisting of SEQ ID NOs: 38, 42, 44, 45, 47, and 50 or a fragment thereof having at least 90% sequence identity with SEQ ID NOs: 38, 42, 44, 45, 47, or 50.

In other embodiments, the PD-L1/CD137 bispecific comprises a light chain sequence selected from the group consisting of SEQ ID NOs: 40 and 46 or a fragment derivative thereof having at least 90%/o sequence identity SEQ ID NOs: 40 or 46.

In some embodiments, the CD137 binding module is a scFv subunit that blocks the CD137/CD137 ligand interaction and possesses crosslinking dependent agonistic activity for CD137 signaling and T cell activation. In some embodiments, the CD137 binding module is a scFv subunit stabilized with a disulfide bond.

In some embodiments, the PD-L1/CD137 bispecific comprises an antibody scaffold module having an IgG format in which a CD137 scFv binding module is fused to the C-terminus of the heavy chain or the light chain of the antibody scaffold module to create a PD-L1/CD137 bispecific.

In some embodiments, the disclosed PD-L1/CD137 bispecific further comprises a Fc region that is engineered to abolish/minimize cross-linking activity with FcγRs, which silence or eliminate Fc-mediated effector functions of T cells.

The present disclosure also provides pharmaceutical compositions comprising or consisting of at least one of the PD-L1/CD137 bispecifics disclosed herein, and optionally a pharmaceutically acceptable diluent, carrier, vehicle and/or excipient. Such a pharmaceutical composition may be used for the treatment of cancer.

The present disclosure also relates to methods for treatment of cancer in a patient comprising administering to the patient a therapeutically effective amount of at least one of the disclosed PD-L1/CD137 bispecifics, alone or in combination with another therapeutic agent.

The present disclosure also provides isolated polynucleotide sequences encoding at least one of the PD-L1/CD137 bispecifics described herein. The present disclosure also provides isolated polynucleotide sequences encoding at least one of the PD-L1/CD137 bispecific sequences described herein.

The present disclosure also provides vectors comprising a polynucleotide of a PD-L1/CD137 bispecific described herein.

The present disclosure also provides vectors comprising at least one of the PD-L1/CD137 bispecific polynucleotide sequences described herein.

The present disclosure also provides cells comprising one of the PD-L1/CD137 bispecific polynucleotide sequences described herein, or one of the above vectors.

In an exemplary embodiment, a PD-L1/TGFβ bispecific is a binding protein that binds PD-L1 and TGFβ and comprises: (a) an antibody scaffold module in an IgG format comprising a first antigen-binding site that binds PD-L1 and a second antigen-binding site that binds PD-L1; (b) at least one first binding module comprising a third antigen-binding site that binds TGFβ.

In an exemplary embodiment, the first antigen-binding site and the second antigen-binding site of the PD-L1/TGFβ bispecific comprise a heavy chain variable region sequence comprising CDR1: SEQ ID NO: 5, CDR2: SEQ ID NO: 6, and CDR3: SEQ ID NO: 7; and a light chain variable region sequence comprising CDR1: SEQ ID NO: 8, CDR2: SEQ ID NO: 9, and CDR3: SEQ ID NO: 10. In an exemplary embodiment, the first antigen-binding site and the second antigen-binding site of the PD-L1/TGFβ bispecific comprise a heavy chain variable region sequence comprising CDR1: SEQ ID NO: 11, CDR2: SEQ ID NO: 12, and CDR3: SEQ ID NO: 13; and a light chain variable region sequence comprising CDR1: SEQ ID NO: 14, CDR2: SEQ ID NO: 9, and CDR3: SEQ ID NO: 15.

In an exemplary embodiment, the antibody scaffold module of the PD-L1/TGFβ bispecific comprises a heavy chain variable region sequence as set forth in SEQ ID NO: 1 or SEQ ID NO: 3; and a light chain variable region sequence as set forth in SEQ ID NO: 2 or SEQ ID NO: 4.

In an exemplary embodiment, the antibody scaffold module of the PD-L1/TGFβ bispecific comprises a heavy chain variable region sequence as set forth in SEQ ID NO: 1 and a light chain variable region sequence as set forth in SEQ ID NO: 2. In an exemplary embodiment, the antibody scaffold module of the PD-L1/TGFβ bispecific comprises a heavy chain variable region sequence as set forth in SEQ ID NO: 3 and a light chain variable region sequence as set forth in SEQ ID NO: 4.

In an exemplary embodiment, the antibody scaffold module of the PD-L1/TGFβ bispecific comprises a heavy chain variable region sequence as set forth in SEQ ID NO: 1, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 2, and a light chain constant region sequence as set forth in SEQ ID NO: 65 or SEQ ID NO: 66. In an exemplary embodiment, the antibody scaffold module of the PD-L1/TGFβ bispecific comprises a heavy chain variable region sequence as set forth in SEQ ID NO: 3, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 4, and a light chain constant region sequence as set forth in SEQ ID NO: 65 or SEQ ID NO: 66.

In an exemplary embodiment, the antibody scaffold module of the PD-L1/TGFβ bispecific comprises a heavy chain sequence as set forth in SEQ ID NO: 42 and a light chain sequence as set forth in SEQ ID NO: 40. In an exemplary embodiment, the antibody scaffold module of the PD-L1/TGFβ bispecific comprises a heavy chain sequence as set forth in SEQ ID NO: 45 and a light chain sequence as set forth in SEQ ID NO: 40.

In an exemplary embodiment, the PD-L1/TGFβ bispecific has one first binding module. In an exemplary embodiment, the PD-L1/TGFβ bispecific has two first binding modules. In an exemplary embodiment, the PD-L1/TGFβ bispecific has an antibody scaffold module which comprises a heavy chain sequence which comprises a C-terminus and a N-terminus, and wherein this antibody scaffold module comprises a light chain sequence which comprises a C-terminus and a N-terminus, and the first binding module is covalently attached to the C-terminus of the antibody scaffold module heavy chain sequence, the C-terminus of the antibody scaffold module light chain sequence, the N-terminus of the antibody scaffold module heavy chain sequence, the N-terminus of the antibody scaffold module light chain sequence, or combinations thereof, optionally wherein the first binding module and the antibody scaffold module are covalently attached to each other directly or through an interlinker. In an exemplary embodiment, the first binding module and the antibody scaffold module of the PD-L1/TGFβ bispecific are covalently attached to each other through an interlinker, and the interlinker is the sequence, from N- to C-terminus, as set forth in SEQ ID NO: 58. In an exemplary embodiment, the first binding module and the antibody scaffold module of the PD-L1/TGFβ bispecific are covalently attached to each other through an interlinker, and the interlinker is the sequence, from N- to C-terminus, as set forth in SEQ ID NO: 59. In an exemplary embodiment, the first binding module of the PD-L1/TGFβ bispecific is covalently attached to the C-terminus of the antibody scaffold module heavy chain sequence. In an exemplary embodiment, the first binding module of the PD-L1/TGFβ bispecific is covalently attached to the C-terminus of the antibody scaffold module light chain sequence. In an exemplary embodiment, when there is more than one first binding module in the PD-L1/TGFβ bispecific, each is covalently attached to a different antibody scaffold module sequence or to a different end of the antibody scaffold module.

In an exemplary embodiment, the first binding module in the PD-L1/TGFβ bispecific comprises the extracellular domain of TGFβRII. In an exemplary embodiment, the extracellular domain of TGFβRII sequence is set forth in SEQ ID NO: 67.

In an exemplary embodiment, the PD-L1/TGFβ bispecific has two first binding modules. In an exemplary embodiment, the heavy chain sequence of the antibody scaffold module and the first binding module of the PD-L1/TGFβ bispecific comprise a sequence set forth in SEQ ID NO: 51; and wherein the light chain sequence of the antibody scaffold module of the PD-L1/TGFβ bispecific comprises a sequence, from N- to C-terminus, as set forth in SEQ ID NO: 40.

In an exemplary embodiment, the antibody scaffold module of the PD-L1/TGFβ bispecific further comprises a constant region. In an exemplary embodiment, the constant region of the antibody scaffold module of the PD-L1/TGFβ bispecific comprises at least one Fc silencing mutation. In an exemplary embodiment, the Fc silencing mutation in the constant region of the antibody scaffold module of the PD-L1/TGFβ bispecific is L234A L235A or N297A. In an exemplary embodiment, the constant region of the antibody scaffold module of the PD-L1/TGFβ bispecific comprises knobs-in-holes (KiH) mutations.

According to some embodiments, the present disclosure provides PD-L1/TGFβ bispecifics (e.g., 1923Ab20) capable of effectively blocking the interactions between PD-L1 and its receptor PD-1 and sequesters TGFβ. The disclosed PD-L1/TGFβ bispecifics comprise all or a part of the amino acid sequences derived from one of the binding proteins that bind PD-L1 disclosed herein (e.g., 1923Ab2 or 1923Ab3) as a PD-L1 antibody scaffold module and an amino acid sequence comprising the full length extracellular domain of TGFβRII or truncations of TGFβRII (e.g., N-terminal or C-terminal truncations) provided that the sequence can bind and neutralize the biological activities of TGFβ. In some embodiments the first binding module appended to the binding protein that binds PD-L1 is a recombinant TGFβ binding protein derived from the ECD of the human TNFβRII receptor.

In some embodiments, the PD-L1/TGFβ bispecific 1923Ab20 comprises an antibody scaffold module having two Fabs from 1923Ab3 that bind PD-L1, a human IgG1 Fc having two Fc constant chains with L234A L235A mutations (SEQ ID NO: 61), and two polypeptides encoding the extracellular domain of TGFβRII (SEQ ID NO: 67) each attached to the C-terminus of each of the Fc constant chains.

In some embodiments, the PD-L1/TGFβ bispecific comprises a heavy and/or light chain sequence disclosed in Table 5. In other embodiments, the PD-L1/GFβ bispecific comprises a heavy and/or light chain sequence disclosed in Table 6.

In some embodiments, the PD-L1/TGFβ bispecific comprises heavy chain sequence comprising SEQ ID NO: 51 and a light chain sequence comprising SEQ ID NO: 40.

In some embodiments, the PD-L1/TGFβ bispecific comprises a heavy chain sequence selected from the group consisting of SEQ ID NOs: 42, 45, and 51 or a fragment thereof having at least 90% sequence identity to SEQ ID NOs: 42, 45, or 51.

In other embodiments, the PD-L1/TGFβ bispecific comprises a light chain sequence selected from the group consisting of SEQ ID NOs: 39 and 40 or a fragment derivative thereof having at least 90% sequence identity to SEQ ID NOs: 39 and 40.

In some embodiments, the PD-L1/TGFβ bispecifics exhibit one or more of the following characteristics, alone or in combination:
(a) is specific for human PD-L1 and binds human TGFβ;
(b) cross-reacts with cynomolgus PD-L1;
(c) disrupts interaction of PD-1 and PD-L1;
(d) dis-inhibits T cell PD-L1 mediated check-point inhibitory signal; or
(e) sequesters human TGFβ;

The present disclosure also provides pharmaceutical compositions comprising or consisting of at least one of the PD-L1/TGFβ bispecifics disclosed herein, and optionally a pharmaceutically acceptable diluent, carrier, vehicle and/or excipient. Such a pharmaceutical composition may be used for the antibody-based immunotherapy of cancer.

The present disclosure also relates to methods for treatment of cancer in a patient comprising administering to the patient a therapeutically effective amount of at least one of the disclosed PD-L1/TGFβ bispecifics, alone or in combination with another therapeutic agent.

The present disclosure also provides isolated polynucleotide sequences encoding at least one of the PD-L1/TGFβ bispecifics described herein. The present disclosure also provides isolated polynucleotide sequences encoding at least one of the PD-L1/TGFβ bispecific sequences described herein.

The present disclosure also provides vectors comprising a polynucleotide of a PD-L1/TGFβ bispecific described herein.

The present disclosure also provides vectors comprising at least one of the PD-L1/TGFβ bispecific polynucleotide sequences described herein.

The present disclosure also provides cells comprising one of the PD-L1/TGFβ bispecific polynucleotide sequences described herein, or one of the above vectors.

The present disclosure also provides a binding protein that binds PD-L1, TGFβ, and CD137, comprising: (a) an antibody scaffold module in an IgG format comprising a first antigen-binding site that binds PD-L1 and a second antigen-binding site that binds PD-L1; (b) at least one first binding module comprising a third antigen-binding site that binds TGFβ; and (c) at least one second binding module comprising a fourth antigen-binding site that binds CD137. In an exemplary embodiment, the PD-L1/TGFβ/CD137 trispecific is constructed in the form of a recombinant protein comprising an antibody scaffold module that binds PD-L1, a first binding module that comprises an amino acid sequence derived from the TGFβ receptor II binding protein that is capable of binding to human TGFβ and neutralizing its activity, and a second binding module that binds CD137.

In an exemplary embodiment, the first antigen-binding site and the second antigen-binding site of the PD-L1/TGFβ/CD137 trispecific comprise (i) a heavy chain variable region sequence comprising CDR1: SEQ ID NO: 5, CDR2: SEQ ID NO: 6, and CDR3: SEQ ID NO: 7; and a light chain variable region sequence comprising CDR1: SEQ ID NO: 8, CDR2: SEQ ID NO: 9, and CDR3: SEQ ID NO: 10. In an exemplary embodiment, the first antigen-binding site and the second antigen-binding site of the PD-L1/TGFβ/CD137 trispecific comprise a heavy chain variable region sequence comprising CDR1: SEQ ID NO: 11, CDR2: SEQ ID NO: 12, and CDR3: SEQ ID NO: 13; and a light chain variable region sequence comprising CDR1: SEQ ID NO: 14, CDR2: SEQ ID NO: 9, and CDR3: SEQ ID NO: 15.

In an exemplary embodiment, the antibody scaffold module of the PD-L1/TGFβ/CD137 trispecific comprises a heavy chain variable region sequence as set forth in SEQ ID NO: 1 or SEQ ID NO: 3; and a light chain variable region sequence as set forth in SEQ ID NO: 2 or SEQ ID NO: 4.

In an exemplary embodiment, the antibody scaffold module of the PD-L1/TGFβ/CD137 trispecific comprises a heavy chain variable region sequence as set forth in SEQ ID NO: 1 and a light chain variable region sequence as set forth in SEQ ID NO: 2. In an exemplary embodiment, the antibody scaffold module of the PD-L1/TGFβ/CD137 trispecific comprises a heavy chain variable region sequence as set forth in SEQ ID NO: 3 and a light chain variable region sequence as set forth in SEQ ID NO: 4.

In an exemplary embodiment, the antibody scaffold module of the PD-L1/TGFβ/CD137 trispecific comprises a heavy chain variable region sequence as set forth in SEQ ID NO: 1, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 2, and a light chain constant region sequence as set forth in SEQ ID NO: 65 or SEQ ID NO: 66. In an exemplary embodiment, the antibody scaffold module of the PD-L1/TGFβ/CD137 trispecific comprises a heavy chain variable region sequence as set forth in SEQ ID NO: 3, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 4, and a light chain constant region sequence as set forth in SEQ ID NO: 65 or SEQ ID NO: 66.

In an exemplary embodiment, the antibody scaffold module of the PD-L1/TGFβ/CD137 trispecific comprises a heavy chain sequence as set forth in SEQ ID NO: 42 and a light chain sequence as set forth in SEQ ID NO: 40. In an exemplary embodiment, the antibody scaffold module of the PD-L1/TGFβ/CD137 trispecific comprises a heavy chain sequence as set forth in SEQ ID NO: 45 and a light chain sequence as set forth in SEQ ID NO: 40.

In an exemplary embodiment, the PD-L1/TGFβ/CD137 trispecific has one first binding module. In an exemplary embodiment, the PD-L1/TGFβ/CD137 trispecific has two first binding modules. In an exemplary embodiment, the PD-L1/TGFβ/CD137 trispecific has an antibody scaffold module which comprises a heavy chain sequence which comprises a C-terminus and an N-terminus, and wherein this antibody scaffold module comprises a light chain sequence which comprises a C-terminus and an N-terminus, and the first binding module is covalently attached to the C-terminus of the antibody scaffold module heavy chain sequence, the C-terminus of the antibody scaffold module light chain sequence, the N-terminus of the antibody scaffold module heavy chain sequence, the N-terminus of the antibody scaffold module light chain sequence, or combinations thereof, optionally wherein the first binding module and the antibody scaffold module are covalently attached to each other directly or through a first binding module interlinker. In an exemplary embodiment, the first binding module and the antibody scaffold module of the PD-L1/TGFβ/CD137 trispecific are covalently attached to each other through a first binding module interlinker, and the first binding module interlinker is the sequence, from N- to C-terminus, as set forth in SEQ ID NO: 58. In an exemplary embodiment, the first binding module and the antibody scaffold module of the PD-L1/TGFβ/CD137 trispecific are covalently attached to each other through a first binding module interlinker, and the first binding module interlinker is the sequence, from N- to C-terminus, as set forth in SEQ ID NO: 59. In an exemplary embodiment, the first binding module of the PD-L1/TGFβ/CD137 trispecific is covalently attached to the C-terminus of the antibody scaffold module heavy chain sequence. In an exemplary embodiment, the first binding module of the PD-L1/TGFβ/CD137 trispecific is covalently attached to the C-terminus of the antibody scaffold module light chain sequence. In an exemplary embodiment, when there is more than one first binding module in the PD-L1/TGFβ/CD137 trispecific, each is covalently attached to a different antibody scaffold module sequence or to a different end of the antibody scaffold module.

In an exemplary embodiment, the first binding module in the PD-L1/TGFβ/CD137 trispecific comprises the extracellular domain of TGFβRII. In an exemplary embodiment, the extracellular domain of TGFβRII in the PD-L1/TGFβ/CD137 trispecific comprises the sequence set forth in SEQ ID NO: 67.

In an exemplary embodiment, the second binding module in the PD-L1/TGFβ/CD137 trispecific is a scFv, which comprises a heavy chain variable region sequence and a light chain variable sequence, wherein the sequences are covalently attached to each other directly or through a scFv fusion linker. In an exemplary embodiment, the scFv fusion linker comprises glycine and serine. In an exemplary embodiment, the scFv fusion linker comprises the sequence Gly-Gly-Gly-Ser. In an exemplary embodiment, the scFv fusion linker comprises the sequence set forth in SEQ ID NO: 58. In an exemplary embodiment, the scFv fusion linker is the sequence set forth in SEQ ID NO: 58. In an exemplary embodiment, the scFv fusion linker comprises the sequence set forth in SEQ ID NO: 59. In an exemplary embodiment, the scFv fusion linker is the sequence set forth in SEQ ID NO: 59. In an exemplary embodiment, the second binding module in the PD-L1/TGFβ/CD137 trispecific comprises a sequence as set forth in SEQ ID NO: 53. In an exemplary embodiment, the second binding module in the PD-L1/TGFβ/CD137 trispecific comprises a sequence as set forth in SEQ ID NO: 54. In an exemplary embodiment, the second binding module in the PD-L1/TGFβ/CD137 trispecific comprises a sequence as set forth in SEQ ID NO: 55. In an exemplary embodiment, the second binding module in the PD-L1/TGFβ/CD137 trispecific comprises a sequence as set forth in SEQ ID NO: 56.

In an exemplary embodiment, the PD-L1/TGFβ/CD137 trispecific has one second binding module. In an exemplary embodiment, the PD-L1/TGFβ/CD137 trispecific has two second binding modules. In an exemplary embodiment, the PD-L1/TGFβ/CD137 trispecific has an antibody scaffold module which comprises a heavy chain sequence which comprises a C-terminus and a N-terminus, and wherein the antibody scaffold module comprises a light chain sequence which comprises a C-terminus and a N-terminus, and the second binding module is covalently attached to the C-terminus of the antibody scaffold module heavy chain sequence, the C-terminus of the antibody scaffold module light chain sequence, the N-terminus of the antibody scaffold module heavy chain sequence, the N-terminus of the antibody scaffold module light chain sequence, or combinations thereof, optionally wherein the second binding module and the antibody scaffold module are covalently attached to each other directly or through a second binding module interlinker.

In an exemplary embodiment, the second binding module and the antibody scaffold module of the PD-L1/TGFβ/CD137 trispecific are covalently attached to each other through a second binding module interlinker, and the second binding module interlinker is set forth in SEQ ID NO: 58. In an exemplary embodiment, the second binding module and the antibody scaffold module of the PD-L1/TGFβ/CD137 trispecific are covalently attached to each other through a second binding module interlinker, and the second binding module interlinker is set forth in SEQ ID NO: 59.

In an exemplary embodiment, the second binding module of the PD-L1/TGFβ/CD137 trispecific is covalently attached to the C-terminus of the antibody scaffold module heavy chain sequence. In an exemplary embodiment, the first binding module of the PD-L1/TGFβ/CD137 trispecific is covalently attached to the C-terminus of the antibody scaffold module light chain sequence. In an exemplary embodiment, the second binding module of the PD-L1/TGFβ/CD137 trispecific is covalently attached to the C-terminus of the antibody scaffold module heavy chain sequence, and the first binding module of the PD-L1/TGFβ/CD137 trispecific is covalently attached to the C-terminus of the antibody scaffold module light chain sequence.

In an exemplary embodiment, the second binding module of the PD-L1/TGFβ/CD137 trispecific is covalently attached to the C-terminus of the antibody scaffold module light chain sequence. In an exemplary embodiment, the first binding module of the PD-L1/TGFβ/CD137 trispecific is covalently attached to the C-terminus of the antibody scaffold module heavy chain sequence. In an exemplary embodiment, the second binding module of the PD-L1/TGFβ/CD137 trispecific is covalently attached to the C-terminus of the antibody scaffold module light chain sequence, and the first binding module of the PD-L1/TGFβ/CD137 trispecific is covalently attached to the C-terminus of the antibody scaffold module heavy chain sequence.

In an exemplary embodiment, when there is more than one second binding module in the PD-L1/TGFβ/CD137 trispecific, each is covalently attached to a different antibody scaffold module sequence or to a different end of the antibody scaffold module (e.g., one binding module may be attached to the C-terminus of the heavy chain in the antibody scaffold module and the other binding module may be attached to the N-terminus of the same heavy chain). In an exemplary embodiment, the PD-L1/TGFβ/CD137 trispecific has one second binding module, and wherein the one second binding module is covalently attached to the C-terminus of the antibody scaffold module heavy chain sequence. In an exemplary embodiment, the PD-L1/TGFβ/CD137 trispecific has one second binding module, and wherein the one second binding module is covalently attached to the C-terminus of the antibody scaffold module light chain sequence. In an exemplary embodiment, the PD-L1/TGFβ/CD137 trispecific has two second binding modules, and wherein one second binding module is covalently attached to the C-terminus of the antibody scaffold module heavy chain sequence, and the other second binding module is covalently attached to the C-terminus of the antibody scaffold module light chain sequence. In an exemplary embodiment, the second binding module of the PD-L1/TGFβ/CD137 trispecific is a scFv.

In an exemplary embodiment, the second binding module of the PD-L1/TGFβ/CD137 trispecific comprises a heavy chain variable region sequence comprising CDR1: SEQ ID NO: 5, CDR2: SEQ ID NO: 22, and CDR3: SEQ ID NO: 23; and a light chain variable region sequence comprising CDR1: SEQ ID NO: 24, CDR2: SEQ ID NO: 25, and CDR3: SEQ ID NO: 26. In an exemplary embodiment, the second binding module of the PD-L1/TGFβ/CD137 trispecific comprises, from a heavy chain variable region sequence comprising CDR1: SEQ ID NO: 27, CDR2: SEQ ID NO: 28, and CDR3: SEQ ID NO: 29; and a light chain variable region sequence comprising CDR1: SEQ ID NO: 30, CDR2: SEQ ID NO: 9, and CDR3: SEQ ID NO: 31. In an exemplary embodiment, the second binding module of the PD-L1/TGFβ/CD137 trispecific comprises a heavy chain variable region sequence comprising CDR1: SEQ ID NO: 32, CDR2: SEQ ID NO: 33, and CDR3: SEQ ID NO: 34; and a light chain variable region sequence comprising CDR1: SEQ ID NO: 35, CDR2: SEQ ID NO: 36, and CDR3: SEQ ID NO: 37. In an exemplary embodiment, the second binding module of the PD-L1/TGFβ/CD137 trispecific comprises a heavy chain variable region sequence as set forth in SEQ ID NO: 16 and a light chain variable region sequence as set forth in SEQ ID NO: 17. In an exemplary embodiment, the second binding module of the PD-L1/TGFβ/CD137 trispecific comprises a heavy chain variable region sequence as set forth in SEQ ID NO: 18 and a light chain variable region sequence as set forth in SEQ ID NO: 19. In an exemplary embodiment, the second binding module of the PD-L1/TGFβ/CD137 trispecific comprises a heavy chain variable region sequence as set forth in SEQ ID NO: 20 and a light chain variable region sequence as set forth in SEQ ID NO: 21.

In an exemplary embodiment, the second binding module of the PD-L1/TGFβ/CD137 trispecific comprises a sequence as set forth in SEQ ID NO: 53. In an exemplary embodiment, the second binding module of the PD-L1/TGFβ/CD137 trispecific comprises a sequence as set forth in SEQ ID NO: 54. In an exemplary embodiment, the second binding module of the PD-L1/TGFβ/CD137 trispecific comprises a sequence as set forth in SEQ ID NO: 55. In an exemplary embodiment, the second binding module of the PD-L1/TGFβ/CD137 trispecific comprises a sequence as set forth in SEQ ID NO: 56.

In an exemplary embodiment, the PD-L1/TGFβ/CD137 trispecific has two first binding modules and two second binding modules. In an exemplary embodiment, the PD-L1/TGFβ/CD137 trispecific comprises the heavy chain sequence of the antibody scaffold module and the second binding module as set forth in SEQ ID NO: 38; and the light chain sequence of the antibody scaffold module and the first binding module as set forth in SEQ ID NO: 39. In an exemplary embodiment, the PD-L1/TGFβ/CD137 trispecific comprises the heavy chain sequence of the antibody scaffold module and the second binding module as set forth in SEQ ID NO: 50; and the light chain sequence of the antibody scaffold module and the first binding module as set forth in SEQ ID NO: 39. In an exemplary embodiment, the PD-L1/TGFβ/CD137 trispecific comprises the heavy chain sequence of the antibody scaffold module and the first binding module as set forth in SEQ ID NO: 51; and the light chain sequence of the antibody scaffold module and the second binding module as set forth in SEQ ID NO: 52.

In an exemplary embodiment, the PD-L1/TGFβ/CD137 trispecific has two first binding modules and one second binding module. In an exemplary embodiment, the PD-L1/TGFβ/CD137 trispecific comprises: the heavy chain sequence of the antibody scaffold module and the second binding module as set forth in SEQ ID NO: 41; the light chain sequence of the antibody scaffold module and the first binding module as set forth in SEQ ID NO: 39, the heavy chain sequence of the antibody scaffold module comprises a sequence set forth in SEQ ID NO: 42; the light chain sequence of the antibody scaffold module and the first binding module comprise a sequence set forth in SEQ ID NO: 39. In an exemplary embodiment, the PD-L1/TGFβ/CD137 trispecific has a structure which is, from N- to C-terminus: the heavy chain sequence of the antibody scaffold module and the second binding module comprise a sequence set forth in SEQ ID NO: 43; the light chain sequence of the antibody scaffold module and the first binding module comprise a sequence set forth in SEQ ID NO: 39, the heavy chain sequence of the antibody scaffold module comprise a sequence set forth in SEQ ID NO: 42; the light chain sequence of the antibody scaffold module and the first binding module comprise a sequence set forth in SEQ ID NO: 39.

In an exemplary embodiment, the antibody scaffold module of the PD-L1/TGFβ/CD137 trispecific further comprises a constant region. In an exemplary embodiment, the constant region of the antibody scaffold module of the PD-L1/TGFβ/CD137 trispecific comprises at least one Fc silencing mutation. In an exemplary embodiment, the Fc silencing mutation in the constant region of the antibody scaffold module of the PD-L1/TGFβ/CD137 trispecific is L234A L235A or N297A. In an exemplary embodiment, the constant region of the antibody scaffold module of the PD-L1/TGFβ/CD137 trispecific comprises a knobs-in-holes (KiH) mutation.

According to some embodiments, the present disclosure provides PD-L1/TGFβ/CD137 trispecifics (e.g., 1923Ab7, 1923Ab9, 1923Ab10, 1923Ab17 and 1923Ab19) that: 1) block the interaction between PD-L1 and its receptor PD-1; 2) possess crosslinking dependent agonistic activity for CD137 signaling and/or 3) neutralizes the immunosuppressive activities of TGFβ.

In some embodiments, the disclosed PD-L1/TGFβ/CD137 trispecifics comprise amino acid sequences derived from one of the binding protein that binds PD-L1 (e.g., 1923Ab2 or 1923Ab3) as an antibody scaffold module and TGFβ binding amino acid sequence derived from the ECD of the human TGFβRII receptor disclosed herein as a first binding module and amino acid sequences derived from one of the binding protein that binds CD137 (e.g., 1923Ab4, 1923Ab5, or 1923Ab6) disclosed herein as a CD137 second binding module.

In some embodiments, the PD-L1/TGFβ/CD137 trispecifics comprise an antibody scaffold module that disrupts the PD-1/PD-L1 binding interaction and dis-inhibits PD-1/PD-L1 checkpoint-mediated inhibition of T cells. The PD-L1 antibody scaffold module may be in the form of an IgG molecule (e.g., 1923Ab7, 1923Ab9, 1923Ab10, 1923Ab17, 1923Ab19).

In some embodiments, the disclosed PD-L1/TGFβ/CD137 trispecifics further comprise a CD137 second binding module that blocks the CD137/CD137 ligand interaction and possesses agonistic activity for CD137 signaling. In alternative embodiments, the CD137 second binding module may comprise a scFv (e.g., 1923Ab7, 1923Ab9, 1923Ab10, 1923Ab17 and 1923Ab19). In alternative embodiments, the disclosed PD-L1/TGFβ/CD137 trispecifics may comprise a second binding module (e.g., a CD137 scFv) fused to the C-terminus of the heavy chain or the light chain of a PD-L1 antibody scaffold module. In alternative embodiments, the disclosed PD-L1/TGFβ/CD137 trispecifics may comprise a second binding module (e.g., a CD137 scFv) fused to the N-terminus of the heavy chain or the light chain of a PD-L1 antibody scaffold module.

In some embodiments, the second binding module that binds CD137 contributes monovalent binding to CD137. In alternative embodiments, the second binding module that binds CD137 contributes bivalent binding to CD137.

In some embodiments, the second binding module is a CD137 scFv and may be stabilized with a disulfide bond.

In some embodiments, the PD-L1/TGFβ/CD137 trispecifics comprise a TGFβ first binding module. In some embodiments the disclosed TGFβ first binding module comprises the extracellular domain of the human TGFβRII receptor. The TGFβ first binding module functions to neutralize the biological activities of TGFβ present in the tumor microenvironment. In alternative embodiments, the TGFβ first binding module comprises truncated versions (e.g., N-terminal or C-terminal truncations) of the human TNFβRII receptor that are capable of binding to human TGFβ.

In alternative embodiments, the disclosed PD-L1/TGFβ/CD137 trispecifics comprise a PD-L1 antibody scaffold module in which TGFβ first binding module(s) are attached via a linker to the C-terminus or to the N-terminus of the heavy chains or the light chains of the PD-L1 antibody scaffold module.

In some embodiments, disclosed PD-L1/TGFβ/CD137 trispecifics comprise an antibody scaffold module having a molecular design that is symmetrical (e.g., 1923Ab7, 1923Ab17 and 1923Ab19). In alternative embodiments, the disclosed PD-L1/TGFβ/CD137 trispecifics are characterized by an asymmetrical design (e.g., 1923Ab9 and 1923Ab10). In order to direct the heterodimerization of the heavy chains of the asymmetrical disclosed PD-L1/TGFβ/CD137 trispecifics, knobs-into-holes (KIHs) technology can be applied.

In some embodiments, the PD-L1/TGFβ/CD137 trispecific 1923Ab7 comprises a PD-L1 antibody scaffold module having two Fabs from 1923Ab3, a human IgG1 Fc having two Fc constant chains with L234A L235A mutations (SEQ ID NO: 61), two second binding modules in the form of a CD137 scFv (VH precedes VL) (SEQ ID NO: 53) derived from 1923Ab4 each separately attached to the C-terminus of each of the two Fc constant chains, and two TGFβ first binding modules as polypeptides encoding the extracellular domain of TGFβRII (SEQ ID NO: 67) each separately attached to the C-terminus of each light chain in the Fabs.

In some embodiments, the PD-L1/TGFβ/CD137 trispecific 1923Ab9 comprises a PD-L1 antibody scaffold module having two Fabs from 1923Ab3, a heterodimeric human IgG1 Fc having two Fc constant chains with L234A L235A mutations and knobs-in-holes (KiH) mutations (e.g., the Fc constant chains are set forth in SEQ ID NOS: 62 and 63), one second binding module in the form of a CD137 scFv (VH precedes VL) (SEQ ID NO: 53) derived from 1923Ab4 attached to the C-terminus of the knob Fc constant chain, and two TGFβ first binding modules as polypeptides encoding the extracellular domain of TGFβRII (SEQ ID NO: 67) each separately attached to the C-terminus of each light chain in the Fabs.

In some embodiments, the PD-L1/TGFβ/CD137 trispecific 1923Ab10 comprises a PD-L1 antibody scaffold module having two Fabs from 1923Ab3, a heterodimeric human IgG1 Fc having two Fc chains with L234A L235A mutations and knobs-in-holes (KiH) mutations (e.g., the Fc constant chains are set forth in SEQ ID NOS: 62 and 63), one second binding module in the form of a CD137 scFv (VL precedes VH) (SEQ ID NO: 54) derived from 1923Ab4 attached to the C-terminus of the knob Fc constant chain, and two TGFβ first binding modules as polypeptides encoding the extracellular domain of TGFβRII (SEQ ID NO: 67) each separately attached to the C-terminus of each light chain in the Fabs.

In some embodiments, the PD-L1/TGFβ/CD137 trispecific 1923Ab17 comprises a PD-L1 antibody scaffold module having two Fabs from 1923Ab3, a human IgG1 Fc having two Fc chains with L234A L235A mutations (SEQ ID NO: 61), two second binding modules in the form of a disulfide bond-stabilized CD137 scFv (VH precedes VL) (SEQ ID NO: 55) derived from 1923Ab4 each separately attached to the C-terminus of each of the Fc chains, and two TGFβ first binding modules as polypeptides encoding the extracellular domain of TGFβRII (SEQ ID NO: 67) each separately attached to the C-terminus of each light chain in the Fabs.

In some embodiments, the PD-L1/TGFβ/CD137 trispecific 1923Ab19 comprises a PD-L1 antibody scaffold module having two Fabs from 1923Ab3, a human IgG1 Fc having two Fc chains with L234A L235A mutations (SEQ ID NO: 61), two TGFβ first binding modules as polypeptides encoding the extracellular domain of TGFβRII (SEQ ID NO: 67) each separately attached to the C-terminus of each light chain in the Fabs, and two CD137 second binding module disulfide bond-stabilized scFvs (VH precedes VL) (SEQ ID NO: 56) derived from 1923Ab4 each separately attached to the C-terminus of each of the Fc chains.

In some embodiments, the PD-L1/TGFβ/CD137 trispecific comprises a PD-L1 antibody scaffold module derived from the heavy and/or light chain sequences disclosed in Table 7. In alternative embodiments the PD-L1/TGFβ/CD137 trispecific comprises a combination of two heavy chains and a light chain sequence paired according to Table 7.

In some embodiments, the PD-L1/TGFβ/CD137 trispecific comprises a heavy chain sequence and a light chain sequence, selected from the following combinations:
(a) a heavy chain sequence comprising SEQ ID NO: 38 and a light chain sequence comprising SEQ ID NO: 39;
(b) a heavy chain sequence comprising SEQ ID NO: 48 and a light chain sequence comprising SEQ ID NO: 49;
(c) a heavy chain sequence comprising SEQ ID NO: 50 and a light chain sequence comprising SEQ ID NO: 39; and
(d) a heavy chain sequence comprising SEQ ID NO: 51 and a light chain sequence comprising SEQ ID NO: 52.

In some embodiments, the PD-L1/TGFβ/CD137 trispecifics comprise two different variable heavy chain sequence (design to heterodimerize using a knob-into-hole format) and a variable light chain sequence, selected from the following combinations: (a) a first heavy chain sequence comprising SEQ ID NO: 41, a second heavy chain sequence comprising SEQ ID NO: 42 and a light chain sequence comprising SEQ ID NO: 39; and (b) a first heavy chain sequence comprising SEQ ID NO: 43, a second heavy chain sequence comprising SEQ ID NO: 42 and a light chain sequence comprising SEQ ID NO: 39.

In some embodiments, the PD-L1/TGFβ/CD137 trispecific comprises a heavy chain sequence selected from the group consisting of SEQ ID NOs: 38, 41, 42, 43, 48, 50 and 51 or an analogue or derivative thereof having at least 90% sequence identity to SEQ ID NOs: 38, 41, 42, 43, 48, 50 or 51.

In other embodiments, the PD-L1/TGFβ/CD137 trispecific comprises a light chain sequence selected from the group consisting of SEQ ID NOs: 39, 49 and 52 or an analogue or derivative thereof having at least 90% sequence identity to SEQ ID NOs: 39, 49 or 52.

In some embodiments, the disclosed PD-L1/TGFβ/CD137 trispecific further comprises a Fc region that is engineered to abolish/minimize cross-linking activity with FcγRs, which silence or eliminate Fc-mediated effector functions of T cells.

In some embodiments, the PD-L1/TGFβ/CD137 trispecific exhibits one or more of the following functional characteristics, alone or in combination:
(a) capable of binding to human PD-L1, CD137 and TGFβ;
(b) cross-reacts with cynomolgus PD-L1 and CD137;
(c) disrupts (e.g., reduces or prevents) interaction of PD-1 and PD-L1;
(d) disrupts (e.g., reduces or prevents) human CD137L binding to CD137; (e) exhibits fast on and fast off properties to CD137;
(f) dis-inhibits T cell PD-L1 mediated check-point inhibitory signal;
(g) inhibits TGFβ signaling and neutralizes it's biological activities;
(h) possess PD-L1 dependent agonistic activity to CD137 signaling;
(i) activates T cells in PD-L1 dependent manner; and
(j) kills PD-L1 expressing tumor cells by activating CD8 T cells.

The present disclosure also provides pharmaceutical compositions comprising or consisting of at least one of the PD-L1/TGFβ/CD137 trispecifics disclosed herein, and optionally a pharmaceutically acceptable diluent, carrier, vehicle and/or excipient. Such a pharmaceutical composition may be used for the treatment of cancer.

The present disclosure also relates to methods for treatment of cancer in a patient comprising administering to the patient a therapeutically effective amount of at least one of the PD-L1/TGFβ/CD137 trispecifics disclosed herein, alone or in combination with another therapeutic agent.

The present disclosure also provides isolated polynucleotide sequences encoding at least one of the PD-L1/TGFβ/CD137 trispecifics described herein. The present disclosure also provides isolated polynucleotide sequences encoding at least one of the PD-L1/TGFβ/CD137 trispecific sequences described herein.

The present disclosure also provides vectors comprising a polynucleotide of a PD-L1/TGFβ/CD137 trispecific described herein.

The present disclosure also provides vectors comprising at least one of the PD-L1/TGFβ/CD137 trispecific polynucleotide sequences described herein.

The present disclosure also provides cells comprising one of the PD-L1/TGFβ/CD137 trispecific polynucleotide sequences described herein, or one of the above vectors.

The present disclosure also provides a binding protein that binds CD137, TGFβ, and PD-L1, comprising: (a) an antibody scaffold module in an IgG format comprising a first antigen-binding site that binds CD137 and a second antigen-binding site that binds CD137; (b) at least one first binding module comprising a third antigen-binding site that binds TGFβ; and (c) at least one second binding module comprising a fourth antigen-binding site that binds PD-L1. In some embodiments, the present disclosure also provides CD137/TGFβ/PD-L1 trispecifics constructed in the form of a recombinant protein comprising an antibody scaffold module that binds CD137, a first binding module that comprises an amino acid sequence derived from the TGFβ receptor II binding protein that is capable of binding to human TGFβ and neutralizing its activity, and a second binding module that binds PD-L1.

In an exemplary embodiment, the first antigen-binding site and the second antigen-binding site of the CD137/TGFβ/PD-L1 trispecific comprises a heavy chain variable region sequence comprising CDR1: SEQ ID NO: 5, CDR2: SEQ ID NO: 22, and CDR3: SEQ ID NO: 23; and a light chain variable region sequence comprising CDR1: SEQ ID NO: 24, CDR2: SEQ ID NO: 25, and CDR3: SEQ ID NO: 26. In an exemplary embodiment, the first antigen-binding site and the second antigen-binding site of the CD137/TGFβ/PD-L1 trispecific comprises a heavy chain variable region sequence comprising CDR1: SEQ ID NO: 27, CDR2: SEQ ID NO: 28, and CDR3: SEQ ID NO: 29; and a light chain variable region sequence comprising CDR1: SEQ ID NO: 30, CDR2: SEQ ID NO: 9, and CDR3: SEQ ID NO: 31. In an exemplary embodiment, the first antigen-binding site and the second antigen-binding site of the CD137/TGFβ/PD-L1 trispecific comprises a heavy chain variable region sequence comprising CDR1: SEQ ID NO: 32, CDR2: SEQ ID NO: 33, and CDR3: SEQ ID NO: 34; and a light chain variable region sequence comprising CDR1: SEQ ID NO: 35, CDR2: SEQ ID NO: 36, and CDR3: SEQ ID NO: 37.

In an exemplary embodiment, the antibody scaffold module of the CD137/TGFβ/PD-L1 trispecific comprises a heavy chain variable region sequence as set forth in SEQ ID NO: 16, SEQ ID NO: 18, or SEQ ID NO: 20. In an exemplary embodiment, the antibody scaffold module of the CD137/TGFβ/PD-L1 trispecific comprises a light chain variable region sequence as set forth in SEQ ID NO: 17; SEQ ID NO: 19, or SEQ ID NO: 21.

In an exemplary embodiment, the antibody scaffold module of the CD137/TGFβ/PD-L1 trispecific comprises a heavy chain variable region sequence as set forth in SEQ ID NO: 16 and a light chain variable region sequence as set forth in SEQ ID NO: 17. In an exemplary embodiment, the antibody scaffold module of the CD137/TGFβ/PD-L1 trispecific comprises a heavy chain variable region sequence as set forth in SEQ ID NO: 18 and a light chain variable region sequence as set forth in SEQ ID NO: 19. In an exemplary embodiment, the antibody scaffold module of the CD137/TGFβ/PD-L1 trispecific comprises a heavy chain variable region sequence as set forth in SEQ ID NO: 20 and a light chain variable region sequence as set forth in SEQ ID NO: 21.

In an exemplary embodiment, the antibody scaffold module of the CD137/TGFβ/PD-L1 trispecific comprises a heavy chain variable region sequence as set forth in SEQ ID NO: 16, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 17, and a light chain constant region sequence as set forth in SEQ ID NO: 65 or SEQ ID NO: 66. In an exemplary embodiment, the antibody scaffold module of the CD137/TGFβ/PD-L1 trispecific comprises a heavy chain variable region sequence as set forth in SEQ ID NO: 18, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 19, and a light chain constant region sequence as set forth in SEQ ID NO: 65 or SEQ ID NO: 66. In an exemplary embodiment, the antibody scaffold module of the CD137/TGFβ/PD-L1 trispecific comprises a heavy chain variable region sequence as set forth in SEQ ID NO: 20, a heavy chain constant region sequence as set forth in SEQ ID NO: 60, SEQ ID NO: 61, SEQ ID NO: 62, SEQ ID NO: 63, or SEQ ID NO: 64, a light chain variable region sequence as set forth in SEQ ID NO: 21, and a light chain constant region sequence as set forth in SEQ ID NO: 65 or SEQ ID NO: 66.

In an exemplary embodiment, the antibody scaffold moiety of the CD137/TGFβ/PD-L1 trispecific comprises a heavy chain sequence as set forth in SEQ ID NO: 75 and a light chain sequence as set forth in SEQ ID NO: 76.

In an exemplary embodiment, the CD137/TGFβ/PD-L1 trispecific has one first binding module. In an exemplary embodiment, the CD137/TGFβ/PD-L1 trispecific has two first binding modules. In an exemplary embodiment, the CD137/TGFβ/PD-L1 trispecific has an antibody scaffold module which comprises a heavy chain sequence which comprises a C-terminus and a N-terminus, and wherein the antibody scaffold module comprises a light chain sequence which comprises a C-terminus and a N-terminus, and the first binding module is covalently attached to the C-terminus of the antibody scaffold module heavy chain sequence, the C-terminus of the antibody scaffold module light chain sequence, the N-terminus of the antibody scaffold module heavy chain sequence, the N-terminus of the antibody scaffold module light chain sequence, or combinations thereof, optionally wherein the first binding module and the antibody scaffold module are covalently attached to each other directly or through a first binding module interlinker. In an exemplary embodiment, the first binding module and the antibody scaffold module of the CD137/TGFβ/PD-L1 trispecific are covalently attached to each other through a first binding module interlinker, and the first binding module interlinker is set forth in SEQ ID NO: 58. In an exemplary embodiment, the first binding module and the antibody scaffold module of the CD137/TGFβ/PD-L1 trispecific are covalently attached to each other through a first binding module interlinker as set forth in SEQ ID NO: 59.

In an exemplary embodiment, the first binding module of the CD137/TGFβ/PD-L1 trispecific is covalently attached to the C-terminus of the antibody scaffold module heavy chain sequence. In an exemplary embodiment, the first binding module of the CD137/TGFβ/PD-L1 trispecific is covalently attached to the C-terminus of the antibody scaffold module light chain sequence. In an exemplary embodiment, when there is more than one first binding module in the CD137/TGFβ/PD-L1 trispecific, each is covalently attached to a different antibody scaffold module sequence or to a different end of the antibody scaffold module sequence.

In an exemplary embodiment, the first binding module in the CD137/TGFβ/PD-L1 trispecific comprises the extracellular domain of TGFβRII. In an exemplary embodiment, the extracellular domain of TGFβRII in the CD137/TGFβ/PD-L1 trispecific comprises the sequence set forth in SEQ ID NO: 67.

In an exemplary embodiment, the second binding module in the CD137/TGFβ/PD-L1 trispecific is a scFv, which comprises a heavy chain variable region sequence and a light chain variable sequence, wherein the sequences are covalently attached to each other directly or through a scFv fusion linker. In an exemplary embodiment, the scFv fusion linker comprises glycine and serine. In an exemplary embodiment, the scFv fusion linker comprises the sequence Gly-Gly-Gly-Ser. In an exemplary embodiment, the scFv fusion linker comprises the sequence set forth in SEQ ID NO: 58. In an exemplary embodiment, the scFv fusion linker is the sequence set forth in SEQ ID NO: 58. In an exemplary embodiment, the scFv fusion linker comprises the sequence set forth in SEQ ID NO: 59. In an exemplary CD137/TGFβ/PD-L1 trispecific embodiment in which a CD137 antibody provides the scaffold moiety, the second binding module is an scFv that binds to PD-L1 comprising SEQ ID NO: 57.

In an exemplary embodiment, the CD137/TGFβ/PD-L1 trispecific has one second binding module. In an exemplary embodiment, the CD137/TGFβ/PD-L1 trispecific has two second binding modules. In an exemplary embodiment, the antibody scaffold module of the CD137/TGFβ/PD-L1 trispecific comprises a heavy chain sequence which comprises a C-terminus and a N-terminus, and wherein the antibody scaffold module comprises a light chain sequence which comprises a C-terminus and a N-terminus, and the second binding module is covalently attached to the C-terminus of the antibody scaffold module heavy chain sequence, the C-terminus of the antibody scaffold module light chain sequence, the N-terminus of the antibody scaffold module heavy chain sequence, the N-terminus of the antibody scaffold module light chain sequence, or combinations thereof, optionally wherein the second binding module and the antibody scaffold module are covalently attached to each other directly or through a second binding module interlinker.

In an exemplary embodiment, the second binding module and the antibody scaffold module of the CD137/TGFβ/PD-L1 trispecific are covalently attached to each other through a second binding module interlinker, and the second binding module interlinker as set forth in SEQ ID NO: 58. In an exemplary embodiment, the second binding module and the antibody scaffold module of the CD137/TGFβ/PD-L1 trispecific are covalently attached to each other through a second binding module interlinker as set forth in SEQ ID NO: 59.

In an exemplary embodiment, the second binding module of the CD137/TGFβ/PD-L1 trispecific is covalently attached to the C-terminus of the antibody scaffold module heavy chain sequence. In an exemplary embodiment, the first binding module of the CD137/TGFβ/PD-L1 trispecific is covalently attached to the C-terminus of the antibody scaffold module light chain sequence. In an exemplary embodiment, the second binding module of the CD137/TGFβ/PD-L1 trispecific is covalently attached to the C-terminus of the antibody scaffold module heavy chain sequence, and the first binding module of the CD137/TGFβ/PD-L1 trispecific is covalently attached to the C-terminus of the antibody scaffold module light chain sequence.

In an exemplary embodiment, the second binding module of the CD137/TGFβ/PD-L1 trispecific is covalently attached to the C-terminus of the antibody scaffold module light chain sequence. In an exemplary embodiment, the first binding module of the CD137/TGFβ/PD-L1 trispecific is covalently attached to the C-terminus of the antibody scaffold module heavy chain sequence. In an exemplary embodiment, the second binding module of the CD137/TGFβ/PD-L1 trispecific is covalently attached to the C-terminus of the antibody scaffold module light chain sequence, and the first binding module of the CD137/TGFβ/PD-L1 trispecific is covalently attached to the C-terminus of the antibody scaffold module heavy chain sequence.

In an exemplary embodiment, when there is more than one second binding module in the CD137/TGFβ/PD-L1 trispecific, each is covalently is covalently attached to a different antibody scaffold module sequence or to a different end of the antibody scaffold module. In an exemplary embodiment, the CD137/TGFβ/PD-L1 trispecific has one second binding module, and the one second binding module is covalently attached to the C-terminus of the antibody scaffold module heavy chain sequence.

In an exemplary embodiment, the CD137/TGFβ/PD-L1 trispecific has one second binding module, and the one second binding module is covalently attached to the C-terminus of the antibody scaffold module light chain sequence. In an exemplary embodiment, the CD137/TGFβ/PD-L1 trispecific has two second binding modules, and one second binding module is covalently attached to the C-terminus of the antibody scaffold module heavy chain sequence, and the other second binding module is covalently attached to the C-terminus of the antibody scaffold module light chain sequence. In an exemplary embodiment, the second binding module of the CD137/TGFβ/PD-L1 trispecific is a scFv.

In an exemplary embodiment, the second binding module of the CD137/TGFβ/PD-L1 trispecific comprises, from N- to C-terminus: a heavy chain variable region sequence comprising CDR1: SEQ ID NO: 5, CDR2: SEQ ID NO: 6, and CDR3: SEQ ID NO: 7; and a light chain variable region sequence comprising CDR1: SEQ ID NO: 8, CDR2: SEQ ID NO: 9, and CDR3: SEQ ID NO: 10. In an exemplary embodiment, the second binding module of the CD137/TGFβ/PD-L1 trispecific comprises, from N- to C-terminus: a heavy chain variable region sequence comprising CDR1: SEQ ID NO: 11, CDR2: SEQ ID NO: 12, and CDR3: SEQ ID NO: 13; and a light chain variable region sequence comprising CDR1: SEQ ID NO: 14, CDR2: SEQ ID NO: 9, and CDR3: SEQ ID NO: 15.

In an exemplary embodiment, the second binding module of the CD137/TGFβ/PD-L1 trispecific comprises, from N- to C-terminus: a heavy chain variable region sequence as set forth in SEQ ID NO: 1 and a light chain variable region sequence as set forth in SEQ ID NO: 2. In an exemplary embodiment, the second binding module of the CD137/TGFβ/PD-L1 trispecific comprises, from N- to C-terminus: a heavy chain variable region sequence as set forth in SEQ ID NO: 3 and a light chain variable region sequence as set forth in SEQ ID NO: 4. In an exemplary embodiment, the second binding module of the CD137/TGFβ/PD-L1 trispecific comprises a sequence as set forth in SEQ ID NO: 57.

In an exemplary embodiment, the CD137/TGFβ/PD-L1 trispecific has two first binding modules and two second binding modules. In an exemplary embodiment, the CD137/TGFβ/PD-L1 trispecific has a structure in which the heavy chain sequence of the antibody scaffold module and the second binding module comprise SEQ ID NO: 48; and the light chain sequence of the antibody scaffold module and the first binding module comprise SEQ ID NO: 49.

In an exemplary embodiment, the antibody scaffold module of the CD137/TGFβ/PD-L1 trispecific further comprises a constant region. In an exemplary embodiment, the constant region of the antibody scaffold module of the CD137/TGFβ/PD-L1 trispecific comprises at least one Fc silencing mutation. In an exemplary embodiment, the Fc silencing mutation in the constant region of the antibody scaffold module of the CD137/TGFβ/PD-L1 trispecific is L234A L235A or N297A. In an exemplary embodiment, the constant region of the antibody scaffold module of the CD137/TGFβ/PD-L1 trispecific comprises a knobs-in-holes (KiH) mutation.

According to some embodiments, the present disclosure provides CD137/TGFβ/PD-L1 trispecifics (1923Ab16) that: 1) block the interaction between CD137 and its ligand; 2) disrupts (e.g., reduces or prevents the interaction of PD-1 and PD-L1; and 3) neutralize the immunosuppressive activities of TGFβ.

In some embodiments, the disclosed CD137/TGFβ/PD-L1 trispecifics comprise amino acid sequences derived from one of the binding protein that binds CD137 (e.g., 1923Ab4, 1923Ab5 or 1923Ab6) as an antibody scaffold module and TGFβ binding amino acid sequence derived from the ECD of the human TGFβRII receptor disclosed herein as a first binding module and amino acid sequences derived from one of the binding proteins that binds PD-L1 (e.g., 1923Ab2, or 1923Ab3) disclosed herein as a PD-L1 second binding module.

In some embodiments, the CD137/TGFβ/PD-L1 trispecifics comprise an antibody scaffold module that blocks the CD137/CD137 ligand interaction and possess agonistic activity for CD137 signaling. The CD137 antibody scaffold module may be in the form of an IgG molecule (e.g., 1923Ab16) or a binding fragment thereof.

In some embodiments, the disclosed CD137/TGFβ/PD-L1 trispecifics further comprise a PD-L1 second binding module that disrupts the PD-1/PD-L1 binding interaction and dis-inhibits PD-1/PD-L1 checkpoint-mediated inhibition of T cells. In alternative embodiments, the PD-L1 second binding module may comprise a scFv (e.g., 1923Ab16). In alternative embodiments, the disclosed CD137/TGFβ/PD-L1 trispecifics may comprise a PD-L1 scFv second binding module fused to the C-terminus of the heavy chain or the light chain of a CD137 antibody scaffold module.

In some embodiments, the PD-L1 second binding module contributes monovalent binding to PD-L1. In alternative embodiments, the PD-L1 second binding module contributes bivalent binding to PD-L1.

In some embodiments, the PD-L1 scFv second binding module may be stabilized with a disulfide bond.

In some embodiments, the disclosed CD137/TGFβ/PD-L1 trispecifics comprise a tumor microenvironment modulator, exemplified herein as a TGFβ first binding module. In some embodiments the disclosed TGFβ first binding module comprises the extracellular domain of the human TGFβRII receptor. The TGFβ first binding module functions to neutralize the biological activities of TGFβ present in the tumor microenvironment. In alternative embodiments, the TGFβ first binding module may comprise truncated versions (e.g., N-terminal or C-terminal truncations) of the human TNFβRII receptor that are capable of binding to human TGFβ.

In alternative embodiments, the disclosed CD137/TGFβ/PD-L1 trispecifics comprise a CD137 antibody binding module in which TGFβ first binding module(s) are attached via a linker to the C-terminus or to the N-terminus of the heavy chains or the light chains of the CD137 antibody binding module.

In some embodiments, CD137/TGFβ/PD-L1 trispecifics have a molecular design that is symmetrical. In alternative embodiments the CD137/TGFβ/PD-L1 trispecifics are characterized by an asymmetrical design. In order to direct the heterodimerization of the heavy chains of the asymmetrical disclosed CD137/TGFβ/PD-L1 trispecifics, knobs-into-holes (KIHs) technology can be applied.

In some embodiments, the CD137/TGFβ/PD-L1 trispecific 1923Ab16 comprises a CD137 antibody scaffold module having two Fabs from 1923Ab4, a human IgG1 Fc having two Fc chains with L234A L235A mutations (SEQ ID NO: 61), two PD-L1 second binding module scFvs (VH precedes VL) (SEQ ID NO: 57) derived from 1923Ab3 each separately attached to the C-terminus of each of the Fc chains, and two TGFβ first binding modules as polypeptides encoding the extracellular domain of TGFβRII (SEQ ID NO: 67) each separately attached to the C-terminus of each light chain in the Fabs.

In some embodiments, the disclosed CD137/TGFβ/PD-L1 trispecific further comprises a Fc region that is engineered to abolish/minimize cross-linking activity with FcγRs, which silence or eliminate Fc-mediated effector functions of T cells.

In some embodiments, the CD137/TGFβ/PD-L1 trispecific exhibits one or more of the following functional characteristics, alone or in combination:
(a) capable of binding to human PD-L1, CD137 and TGFβ;
(b) cross-reacts with cynomolgus PD-L1 and CD137;
(c) disrupts (e.g., reduces or prevents) interaction of PD-1 and PD-L1;
(d) disrupts (e.g., reduces or prevents) human CD137L binding to CD137;
(e) exhibits fast on and fast off properties to CD137;
(f) dis-inhibits T cell PD-L1 mediated check-point inhibitory signal;
(g) inhibits TGFβ signaling and neutralizes it's biological activities;
(h) possess PD-L1 dependent agonistic activity to CD137 signaling;
(i) activates T cells in PD-L1 dependent manner; and
(j) kills PD-L1 expressing tumor cells by activating CD8 T cells.

The present disclosure also provides pharmaceutical compositions comprising or consisting of at least one of the CD137/TGFβ/PD-L1 trispecifics disclosed herein, and optionally a pharmaceutically acceptable diluent, carrier, vehicle and/or excipient. Such a pharmaceutical composition may be used for the antibody-based immunotherapy of cancer.

The present disclosure also relates to methods for treatment of cancer in a patient comprising administering to the patient a therapeutically effective amount of at least one of the CD137/TGFβ/PD-L1 trispecifics disclosed herein, alone or in combination with another therapeutic agent.

The present disclosure also provides isolated polynucleotide sequences encoding at least one of the CD137/TGFβ/PD-L1 trispecifics described herein. The present disclosure also provides isolated polynucleotide sequences encoding at least one of the CD137/TGFβ/PD-L1 trispecific sequences described herein.

The present disclosure also provides vectors comprising a polynucleotide of a CD137/TGFβ/PD-L1 trispecific described herein.

The present disclosure also provides vectors comprising at least one of the CD137/TGFβ/PD-L1 trispecific polynucleotide sequences described herein.

The present disclosure also provides cells comprising one of the CD137/TGFβ/PD-L1 trispecific polynucleotide sequences described herein, or one of the above vectors.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIGS. 1A-K provide the amino acid sequences of the VH and VL domains of the human binding proteins that bind PD-L1 or that bind CD137, the HC and LC sequences of the PD-L1/CD137 bispecifics, PD-L1/TGFβ bispecifics, and PD-L1/CD137/TGFβ trispecifics and the scFv subunits used to prepare the disclosed trispecifics. The CDR sequences (Kabat numbering) of the anti-PD-L1 and anti-CD137 are underlined in their respective variable domain sequences. Sequence identifiers are provided.

FIGS. 2A-B show the binding activity of PD-L1 monospecifics, A) 1923Ab2 and B) 1923Ab3, to the human, mouse and cyno PD-L1 proteins by ELISA.

FIG. 3 shows the binding activity of PD-L1 monospecifics in the human PD-L1-expressing HEK293T by an image binding assays.

FIGS. 4A-B show the activity of PD-L1 monospecifics, A) 1923Ab2 and B) 1923Ab3, to block interaction of PD-L1 and PD-1 by PD-1/PD-L1 blockage reporter assay.

FIGS. 5A-C show the binding activities of CD137 monospecifics to the human, mouse and cyno CD137-expressing HEK293T cells by image binding assays.

FIG. 6 shows the comparison of binding activities of CD137 antibodies to the human, CD137-expressing HEK293T cells by image binding assay.

FIG. 7 shows the activity of CD137 monospecifics to block CD137L binding to CD137 by biolayer interferometry (BLI).

FIG. 8 shows the effect of cross-linking of CD137 monospecifics on HEK293T CD137 reporter cells, expressing human CD137 and the NFκB luciferase reporter.

FIG. 9 shows the effect of cross-linking of CD137 monospecifics on human PBMCs stimulated with anti-CD3 to induce IFNγ secretion.

FIG. 10A shows the schematic diagram of four human/mouse hybrid CD137 expression constructs. Human CRD regions were replaced by their mouse counterpart and transiently expressed on the HEK293T cells. FIGS. 10B-F show the binding activity of Urelumab-NR (PC2), Utomilumab-NR (PC3) and 1923Ab4 to human CD137 wild type (10B) and human/mouse hybrid CD137 proteins msCRD1 (10C), msCRD2 (10D), msCRD3 (10E) and msCRD4 (10F).

FIG. 11A shows the sequence alignment of CRD4 domain of human and mouse CD137. Five expression constructs of human CD137 were generated by changing human amino acid sequence into mouse amino acid sequence as indicated by M1-M5 and transiently expressed on the HEK293T cells. FIGS. 11B-G show the binding activity of Urelumab-NR (PC2) and 1923Ab4 to human CD137 WT (11B), and the five mutated human CD137 proteins M1 (11C), M2 (11D), M3 (11E), M4 (11F) and M5 (11G).

FIG. 12 shows the epitope regions (depicted as shaded bars) of 1923Ab4 on human CD137 identified by HDX-MS. Boxed regions define the CD137 cysteine-rich domain (CRD) regions.

FIGS. 13A-13L illustrate the structural features of disclosed bispecifics and trispecifics: 1923Ab7 (A); 1923Ab8 (B), 1923Ab9 (C), 1923Ab10 (D), 1923Ab11 (E), 1923Ab12 (F), 1923Ab13 (G), 1923Ab16 (H), 1923Ab17 (I), 1923Ab18 (J), 1923Ab19 (K) and 1923Ab20 (L).

FIGS. 14A-14B describe the heavy chain and light chains comprising the disclosed bispecifics (A) and disclosed trispecifics (B).

FIG. 15 provides a detailed description of the structural and functional subcomponents of the antibody scaffold modules and binding modules used to construct the binding proteins.

FIGS. 16A-16B show binding activity of bispecific and trispecific antibodies in the human CD137-expressing HEK293T by an image binding assay (A) and flow cytometry (B).

FIGS. 17A-17B show the agonist activity of bispecifics and trispecifics to CD137 signaling using HEK293T CD137 reporter cells (A) and Jurkat T CD137 reporter cells (B).

FIGS. 18A-18B show target cells dependent activation of CD137 signaling by bispecifics and trispecifics using Jurkat T CD137 reporter cells in the presence of target cells (A) or the absence of target cells (B).

FIGS. 19A-19B show target cell-dependent activation CD137 signaling of (A) trispecifics and (B) bispecifics generated by fusing scFv of CD137 at different regions of an antibody. CD137 signaling activity was evaluated using the Jurkat T cell CD137 reporter cells.

FIG. 20 shows the activity of bispecifics and trispecifics to block the interaction of PD-L1 and PD-1 by PD-1/PD-L1 blockage reporter assay.

FIG. 21 shows the inhibitory activity of trispecifics to block TGFβ induced signaling by TGFβ blockage reporter assay.

FIGS. 22A-22B show the effect of bispecifics and trispecifics on human PBMCs stimulated with anti-CD3 to induce IFNγ secretion.

FIGS. 23A-23B show (A) T cell mediated killing activity and (B) Induction of IFNγ secretion of bispecifics and trispecifics on human CD8 T cells co-cultured with NUGC4 tumor cells expressing endogenous PD-L1.

FIG. 24 shows the activity of antigen-specific T cell activation of bispecifics and trispecifics on human PBMCs stimulated with CMV lysate in the CMV recall assay. Secreted level of IFNγ was measured as an indication of T cell activation.

FIG. 25 shows the tumor growth in MC38-h-PD-L1 tumor-bearing hCD137 and hDP-L1 double knock-in mice upon treatment with 1923Ab18 or vehicle control. The one-way ANOVA analysis of the tumor sizes from different treatment groups on Day 21 was plotted.

FIG. 26A-26E show the analysis of tumor-infiltrating lymphocytes from MC38-h-PD-L1 tumor-bearing hCD137 and hDP-L1 double knock-in mice upon treatment with 1923Ab18 or vehicle control. The percentages of (A) CD3+CD45+, (B) CD4+CD3+, (C) CD8+CD3+, (D) Treg in CD3+, and (E) CD8/Treg ratio were summarized.

### DETAILED DESCRIPTION

PD-1 and its ligands programmed death-ligand-1 and programmed death-ligand-2 (PD-L1 and PD-L2) act as co-inhibitory factors that regulate the balance between T cell activation, tolerance and immunopathology. Targeting the PD-1/PD-L1 signaling axis is an area of intense therapeutic exploration. The present disclosure provides binding proteins that bind PD-L1, including binding proteins that bind PD-L1 (PD-L1 monospecific), binding proteins that bind PD-L1 and CD137 (PD-L1/CD137 bispecific), binding proteins that bind PD-L1 and TGFβ (PD-L1/TGFβ bispecific), and binding proteins that bind PD-L1, TGFβ, and CD137 and (PD-L1/TGFβ/CD137 trispecific) that can be used for the treatment of cancer. Advantageously, the binding proteins disclosed herein allow for inhibition of PD-L1, result in lower dose formulations, result in less frequent and/or more effective dosing, and lead to reduced cost and increased efficiency. So that the disclosure may be more readily understood, certain technical and scientific terms are specifically defined below. Unless specifically defined elsewhere in this document, all other technical and scientific terms used herein have the meaning commonly understood by one of ordinary skill in the art to which this disclosure belongs.

Throughout this disclosure the following abbreviations will be used:
mAb or Mab or MAb—Monoclonal antibody.
CDR—Complementarity determining region in the immunoglobulin variable regions.
VH or VH—Immunoglobulin heavy chain variable region.
VL or VL—Immunoglobulin light chain variable region.
Fc or Fc region—Constant region of an immunoglobulin heavy chain comprising CH2 and CH3 domains and part of the hinge region.
Fab or Fab fragment—Monovalent antigen binding fragment of an antibody consisting of the VH, CH1 and VL, CL regions.
FR—Antibody framework region, the immunoglobulin variable regions excluding the CDR regions

As described herein, the term “PD-L1” includes variants, isoforms, homologs, orthologs, and paralogs. For example, antibodies specific for a human PD-L1 protein may, in certain cases, cross-react with a PD-L1 protein from a species other than human. In other embodiments, the antibodies specific for a human PD-L1 protein may be particular for the human PD-L1 protein and may exhibit species or other types of cross-reactivity, or may cross-react with PD-L1 from certain other species but not all other species (e.g., cross-react with monkey PD-L1, but not mouse PD-L1). The term “human PD-L1” refers to human sequence PD-L1 such as the complete amino acid sequence of human PD-L1 having NCBI Accession No. NP_054862. PD-L1 is a member of the B7 protein family and shares approximately 20% amino acid sequence identity with B7.1 and B7.2. Human PD-L1 shares 70% and 93% amino acid sequence identity with murine and cynomolgus PD-L1 orthologs, respectively.

As used herein the term “PD-1”, “PD1,” “Programmed cell death protein 1,” “CD279,” and “cluster of differentiation 279” (e.g., Genebank Accession Number NP_005009 (human)) is meant a type I membrane protein that is a member of the extended CD28/CTLA-4 family of T cell regulators. PD1 includes an extracellular IgV domain followed by a transmembrane region and an intracellular tail PD1 is expressed on the surface of activated T cells, B cells and macrophages.

The term “CD137” refers to 4-1BB, or TNFRSF9 (TNF Receptor Superfamily Member 9), is a member of TNF-receptor superfamily (TNFRSF) and is a co-stimulatory molecule which is expressed following the activation of immune cells, both innate and adaptive immune cells. As used herein, 4-1BB may be originated from a mammal, for example, Homo sapiens (human) (NCBI Accession No. NP_001552). As described herein, the term CD137 includes variants, isoforms, homologs, orthologs, and paralogs. For example, antibodies specific for a human CD137 protein may, in certain cases, cross-react with a CD137 protein from a species other than human. In other embodiments, the antibodies specific for a human CD137 protein may be completely specific for the human CD-137 protein and may exhibit species or other types of cross-reactivity, or may cross-react with CD137 from certain other species but not all other species (e.g., cross-react with monkey CD137, but not mouse 4-1BB). The term “cyno CD137” refers to cynomolgus monkey CD137, such as the complete amino acid sequence having NCBI Accession No. XP_005544945.1. The term “mouse CD137” refers to mouse sequence 4-1BB, such as the complete amino acid sequence of mouse 4-1BB having NCBI Accession No. NP_035742.1. The human CD137 sequence in the disclosure may differ from human CD137 of NCBI Accession No. NP_001552 by having, e.g., conserved mutations or mutations in non-conserved regions and the CD137 in the disclosure has substantially the same biological function as the human CD137 of NCBI Accession No. NP_001552.

As used herein the term “transforming growth factor beta,” “TGF-beta,” or “TGFβ” can refer to any TGF-beta protein, including, but not limited to, TGF-beta 1, TGF-beta 2, and TGF-beta 3, including naturally occurring TGF-beta proteins and synthetic proteins, including variants and mimetics. TGF-beta proteins are members of a superfamily of structurally similar regulatory proteins, including, but not limited to, the mammalian TGF-β-1, 2, and 3, inhibin, activin and bone morphogenic proteins. Mature TGF-beta typically exists as a homodimer, such as the dimeric mature TGF-beta molecule, containing two covalently associated TGF-beta molecules.

As used herein the term “TGFβ Receptor II” (“TGFβRII”) is meant a polypeptide having the wild-type human TGFβ Receptor Type 2 Isoform A sequence or Isoform B sequence, or a portion thereof that binds TGFβ (e.g., the amino acid sequence of NCBI Reference Sequence (RefSeq) Accession No. NP_001020018 or NP_003233.4, respectively), including, for example, SEQ ID NO: 74, or having a sequence substantially identical the amino acid sequence of SEQ ID NO: 74. The TGFβRII may retain at least 0.1%, 0.5%, 1%, 5%, 10%, 25%, 35%, 50%, 75%, 90%, 95%, or 99% of the TGFβ-binding activity of the wild-type sequence. The polypeptide of expressed TGFβRII lacks the signal sequence.

The term “antibody” herein is used in the broadest sense and encompasses various antibody structures, including but not limited to monoclonal antibodies, polyclonal antibodies, bispecific antibodies, and trispecific antibodies.

The term “antibody scaffold module” herein refers to a Y-shaped antibody having two heavy and two light chains. The two heavy chains are linked to each other by disulfide bonds and each heavy chain is linked to a light chain by a disulfide bond. An antibody scaffold may have one or more binding modules attached to one or more of its heavy and/or light chains. The antibody binding scaffold comprises two Fabs and a Fc portion having two constant region sequences.

An exemplary antibody such as an IgG comprises two heavy chains and two light chains. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino terminus to carboxy-terminus in the following order: FR1, CDR1, FR2, CDR2, FR3, CDR3, FR4.

The terms “monoclonal antibody” or “mAb” as used herein refers to an antibody obtained from a population of substantially homogeneous antibodies, e.g., the individual antibodies comprising the population are identical and/or bind the same epitope, except for possible variant antibodies, e.g., containing naturally occurring mutations or arising during production and/or storage of a monoclonal antibody preparation. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric antibody” refers to a recombinant antibody in which a portion of the heavy and/or light chain is identical with or homologous to corresponding sequences in antibodies derived from a particular species, or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. In addition, complementarity determining region (CDR) grafting may be performed to alter certain properties of the antibody molecule including affinity or specificity. Typically, the variable domains are obtained from an antibody from an experimental animal (the “parental antibody”), such as a rodent, and the constant domain sequences are obtained from human antibodies, so that the resulting chimeric antibody can direct effector functions in a human subject and will be less likely to elicit an adverse immune response than the parental (e.g., mouse) antibody from which it is derived.

A “human antibody” is an antibody that possesses an amino-acid sequence corresponding to that of an antibody produced by a human and/or has been made using any of the techniques for making human antibodies known to one of skill in the art. This definition of a human antibody specifically excludes a humanized antibody comprising non-human antigen-binding residues. Human antibodies can be produced using various techniques known in the art, including methods described in Cole et al, Monoclonal Antibodies and Cancer Therapy, Alan R. Liss, p. 77 (1985); Boerner et al, J. Immunol, 147(I):86-95 (1991). See also van Dijk and van de Winkel, Curr. Opin. Pharmacol, 5: 368-74 (2001). Human antibodies can be prepared by administering the target antigen to a transgenic animal that has been modified to produce such antibodies in response to antigenic challenge, but whose endogenous loci have been disabled, e.g., immunized HuMab mice (see, e.g., Nils Lonberg et al., 1994, Nature 368:856-859, WO 98/24884, WO 94/25585, WO 93/1227, WO 92/22645, WO 92/03918 and WO 01/09187 regarding HuMab mice), Xenomice (see, e.g., U.S. Pat. Nos. 6,075,181 and 6,150,584 regarding XENOMOUSE™ technology) or Trianni mice (see, e.g., WO 2013/063391, WO 2017/035252 and WO 2017/136734).

The term “humanized antibody” refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those skilled in the art will be aware of humanized antibodies and will also be aware of suitable techniques for their generation. See for example, Hwang, W. Y. K., et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and Selick et al., WO 90/07861, each of which is incorporated herein by reference in its entirety.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, CL and CH1 domains; (ii) F(ab′)2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341: 544-546), which consist of a VH domain; (vi) isolated complementarity determining regions (CDR), and (vii) combinations of two or more isolated CDRs which may optionally be joined by a synthetic linker.

“Complementarity determining region” or “CDR” as the terms are used herein refer to short polypeptide sequences within the variable region of both heavy and light chain polypeptides that are primarily responsible for mediating specific antigen recognition. There are three CDRs (termed CDR1, CDR2, and CDR3) within each VL and each VH. Unless stated otherwise herein, CDR and framework regions are annotated according to the Kabat numbering scheme (Kabat E. A., et al., 1991, Sequences of proteins of Immunological interest, In: NIH Publication No. 91-3242, US Department of Health and Human Services, Bethesda, Md).

In other embodiments, the CDRs of an antibody can be determined according to MacCallum R M et al, (1996) J Mol Biol 262: 732-745, herein incorporated by reference in its entirety. In other embodiments, the CDRs of an antibody can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety. CDRs may also be defined by sequence comparison in Kabat et al., 1991, In: Sequences of Proteins of Immunological Interest, 5^{th} Ed. Public Health Service, National Institutes of Health, Bethesda, Md., whereas HVLs are structurally defined according to the three-dimensional structure of the variable domain, as described by Chothia and Lesk, 1987, J. Mol. Biol. 196: 901-917. Where these two methods result in slightly different identifications of a CDR, the structural definition is preferred. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. IMGT and NORTH provide alternative definitions of the CDRs (see, Lefranc M P. Unique database numbering system for immunogenetic analysis. Immunol Today (1997) 18:509; and North B, Lehmann A, Dunbrack R L J. A new clustering of antibody CDR loop conformations. J Mol Biol. (2011) 406:228-56). Additionally, CDRs may be defined per the Chemical Computing Group (CCG) numbering (Almagro et al., Proteins 2011; 79:3050-3066 and Maier et al, Proteins 2014; 82:1599-1610). The CDR1, CDR2, CDR3 of the heavy and light chains therefore define the unique and functional properties specific for a given antibody.

The “variable domain” (V domain) of an antibody mediates binding and confers antigen specificity of a particular antibody. However, the variability is not evenly distributed across the 110-amino acid span of the variable domains. Instead, the V regions consist of relatively invariant stretches called framework regions (FRs) of 15-30 amino acids separated by shorter regions of extreme variability referred to herein as “hypervariable regions” or CDRs that are each 9-12 amino acids long. As will be appreciated by those in the art, the exact numbering and placement of the CDRs can be different among different numbering systems. However, it should be understood that the disclosure of a variable heavy and/or variable light sequence includes the disclosure of the associated CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3).

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody.

A “single-chain variable fragment” or “scFv” refers to a fusion protein of the variable regions of the heavy (V_{H}) and light chains (V_{L}) of immunoglobulins. For a review of sFv, see Pluckthun in The Pharmacology of Monoclonal Antibodies, vol. 113, Rosenburg and Moore eds., Springer-Verlag, New York, pp. 269-315 (1994). In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. Disulfide-stabilized scFv can be engineered by introducing paired cysteines by mutating specific residues in VH or VL. These residues are at the interface of VH and VL. Please see reference Weatherill, E. E. et al. Towards a universal disulphide stabilised single chain Fv format: importance of interchain disulphide bond location and vL-vH orientation. Protein Eng Des Sel 25, 321-329, NovaRock used VH44-VL100 in the examples.

“Framework” or “framework region” or “FR” refers to variable domain residues other than hypervariable region (HVR) residues. The FR of a variable domain generally consists of four FR domains: FR1, FR2, FR3, and FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In one embodiment, for the VL, the subgroup is subgroup kappa I as in Kabat et al., supra. In one embodiment, for the VH, the subgroup is subgroup Ill as in Kabat et al., supra.

The “hinge region” is generally defined as stretching from 216-238 (EU numbering) or 226-251 (Kabat numbering) of human IgG1. The hinge can be further divided into three distinct regions, the upper, middle (e.g., core), and lower hinge.

The terms “Fc region” and “constant region” are used herein to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, the numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md. (1991).

The term “effector functions,” deriving from the interaction of an antibody Fc region with certain Fc receptors, include but are not necessarily limited to Clq binding, complement dependent cytotoxicity (CDC), FcyR-mediated effector functions such as antibody-dependent cellular cytotoxicity (ADCC), antibody-dependent cell-mediated phagocytosis (ADCP), and downregulation of a cell surface receptor. Such effector functions generally require the Fc region to be combined with an antigen-binding domain (e.g., an antibody variable domain).

The term “T cell-dependent cellular cytotoxicity” (TDCC) describes a series of events when a molecule simultaneously binds to tumor cells and engages cytotoxic T cells and redirects cytolysis by bringing T cells and target cells in close proximity. Activation of ADCC and TDCC both result in the killing of target cells. However, there are some major differences between these two distinct types of cytotoxicity. ADCC effect is mediated through Fcγ receptors expressed on natural killer (NK) cells to bind to constant region of antibody attached on the surface of target cells. TDCC effect is mediated through engaging cytotoxic T cells in close proximity to target cells.

The term “Fc receptor” or “FcR” describes an antibody receptor that binds the Fc region of an immunoglobulin, which is involved in antigen recognition located at the membrane of certain immune cells including B lymphocytes, natural killer cells, macrophages, neutrophils, and mast cells. Fc receptors recognizing the Fc portion of IgG are called Fc gamma receptors (FcγRs). The FcγR family includes allelic variants and alternatively spliced forms of these receptors. Based on the differences in structure, function, and affinity for IgG binding, FcγRs are classified into three major groups: FcγRI, FcγRII (FcγRIIa and FcγRIIb) and FcγRIII (FcγRIIIa and FcγRIIIb). Among them, FcγRI (CD64), FcγRIIa (CD32a), and FcγRIIIa (CD16a) are activating receptors containing the signal transduction motif, immunoreceptor tyrosine-based activation motif (ITAM), in the γ subunit of FcγRI and FcγRIIIa, or in the cytoplasmic tail of FcγRIIa. After binding of antigen-antibody complexes the activatory Fcγ receptors (human: FcγRI, FcγRIIA, FcγRIIC, FcγRIIIA, FcγRIIIB and murine: FcγRI, FcγRIII, FcγRIV) trigger immune effector functions. In contrast, FcγRIIb (CD32b) is an inhibitory receptor. Cross-linking of FcγRIIb leads to the phosphorylation of the immunoreceptor tyrosine-based inhibitory motif (ITIM) and inhibitory signaling transduction (Patel et al. Front Immunol. 2019; 10: 223).

The term “Fc silenced” refers to Fc region that is engineered to minimize/abolish binding activity with FcγRs and complement, leading to silence or eliminate Fc-mediated effector functions. The strategies for engineering Fc include modification of Fc glycosylation, use hybrid of IgG subclasses, or introducing one or more mutations in the hinge and/or CH2 regions. The residues important for effector functions and respective mutations that silence Fc are known in the art, for example, Strohl, W R and Strohl L M, “Antibody Fc engineering for optimal antibody performance” In Therapeutic Antibody Engineering, Cambridge: Woodhead Publishing (2012), pp 242, International Patent Publication No. WO2017008169A1 and WO2021055669.

Specific, non-limiting examples for sites that can be engineered to silence human IgG1 Fc include L234, L235, G237, D265, N297, P329, P331, all in EU numbering.

As used herein, the term “T regulatory cell,” or “Treg” refers to a cell of the immune system that has a regulatory role by suppressing/inhibiting the proliferation, activation and cytotoxic capacity of other immune cells such as CD8 positive (CD8+) effector T cells. Regulatory T cells (Tregs) are characterized by the expression of the master transcription factor forkhead box P3 (Foxp3). There are two major subsets of Treg cells, “natural” Treg (nTreg) cells that develop in the thymus, and “induced” Treg (iTreg) cells that arise in the periphery from CD4+ Foxp3-conventional T cells. Natural Tregs are characterized as expressing both the CD4 T cell co-receptor and CD25, which is a component of the IL-2 receptor. Treg is thus CD4+ CD25+. Expression of the nuclear transcription factor Forkhead box P3 (FoxP3) is the defining property which determines natural Treg development and function. Treg cells exert their suppressive effects by numerous modes of action including suppression by: secretion of inhibitory cytokines (e.g., IL-10, TGFβ, IL-35), modulation of dendritic cell function/maturation, expression of immunoregulatory surface molecules (e.g., CTLA-4, LAG-3) or cytolysis (e.g., granzyme A- and or B-mediated).

As used herein, the term “bispecific” refers to binding proteins comprising an antibody scaffold module and a first binding module, wherein the modules are derived from antibodies and/or receptor proteins that have binding specificities for two different antigens. In one embodiment, the antibody scaffold module has binding specificity for PD-L1, and the first binding module has binding specificity for any other antigen, e.g., for a cell-surface protein, receptor, receptor subunit, tissue-specific antigen, tumor microenvironment modulator, cytokine, etc. In another embodiment, the antibody scaffold module has binding specificity for CD137, and the first binding module has a binding specificity for any other antigen, e.g., for a cell-surface protein, receptor, receptor subunit, tissue-specific antigen, tumor microenvironment modulator, cytokine, etc.

As used herein, the term “trispecific” refers to binding proteins comprising an antibody scaffold module and a first binding module and a second binding module, wherein the modules are derived from antibodies and/or receptor proteins that have binding specificities for three different antigens. In one embodiment, the antibody scaffold module has a binding specificity for PD-L1, and the first and second binding modules have binding specificities for any other antigen (aside from the other binding module's antigen), e.g., for a cell-surface costimulatory receptor (including but not limited to CD137), receptor, receptor subunit, tissue-specific antigen, tumor microenvironment modulator, cytokine, etc. In one embodiment, the antibody scaffold module has a binding specificity for CD137, and the first and second binding modules have binding specificities for any other antigen (aside from the other binding module's antigen), e.g., for a cell-surface costimulatory receptor (including but not limited to PD-L1), receptor, receptor subunit, tissue-specific antigen, tumor microenvironment modulator, cytokine, etc.

With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of 10-4 M or lower, alternatively 10-5 M or lower, alternatively 10-6 M or lower, alternatively 10-7 M or lower, alternatively 10-8 M or lower, alternatively 10-9 M or lower, alternatively 10-10 M or lower, alternatively 10-11 M or lower, alternatively 10-12 M or lower or a Kd in the range of 10-4 M to 10-6 M or 10-6 M to 10-10 M or 10-7 M to 10-9 M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope. As used herein the terms “specific binding,” “specifically binds,” and “selectively binds,” refer to antibody binding to an epitope of CD137, PDL1 and/or TGFβ.

The term “affinity,” as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

An “epitope” is a term of art that indicates the site or sites of interaction between an antibody and its antigen(s). As described by (Janeway, C, Jr., P. Travers, et al. (2001). Immunobiology: the immune system in health and disease. Part II, Section 3-8. New York, Garland Publishing, Inc.): “An antibody generally recognizes only a small region on the surface of a large molecule such as a protein . . . [Certain epitopes] are likely to be composed of amino acids from different parts of the [antigen] polypeptide chain that have been brought together by protein folding. Antigenic determinants of this kind are known as conformational or discontinuous epitopes because the structure recognized is composed of segments of the protein that are discontinuous in the amino acid sequence of the antigen but are brought together in the three-dimensional structure. In contrast, an epitope composed of a single segment of polypeptide chain is termed a continuous or linear epitope” (Janeway, C. Jr., P. Travers, et al. (2001). Immunobiology: the immune system in health and disease. Part II, Section 3-8. New York, Garland Publishing, Inc.).

The term “KD”, as used herein, is intended to refer to the dissociation constant of a particular antibody-antigen interaction. It is calculated by the formula: Koff/Kon=KD

The term “IC₅₀”, as used herein, is intended to refer to the effective concentration of a binding protein disclosed herein needed to neutralize 50% of the bioactivity of an antigen to which it binds.

“EC₅₀” with respect to an agent and a particular activity (e.g. binding to a cell, inhibition of enzymatic activity, activation or inhibition of an immune cell), refers to the efficient concentration of the agent which produces 50% of its maximum response or effect with respect to such activity. “EC₁₀₀” with respect to an agent and a particular activity refers to the efficient concentration of the agent which produces its substantially maximum response with respect to such activity.

As used herein the term “T-cell exhaustion” is defined as an impaired capacity of T cells to proliferate and secrete cytokines, caused by prolonged antigenic stimulation-induced overexpression of immune checkpoint receptors, such as PD-1, CTLA-4, T-cell Ig and mucin-domain containing (TIM)-3, and lymphocyte-activation gene 3.

As used herein the term a “co-stimulatory receptor” on T cells refers to cell surface molecules that can positively induce signaling to fully activate T cells with TCR signaling and cytokine stimulation. Co-signaling pathways play critical roles in T-cell priming and activation, and in modulating T-cell differentiation, effector function, and survival. Co-stimulatory receptors are commonly categorized into 2 groups: the Ig receptor superfamily (IgSF) and the TNF receptor superfamily (TNFRSF).

The term “binding module” refers to any substance that binds PD-L1, CD137, TGFβ, or any other target, which may enhance specific activity of the binding protein of the present invention in comparison to the scaffold module per se. Non-limiting examples of binding modules include anticalin, repebody, monobody, scFv, Fab, scFab, affibody, fynomer, DARPin, nanobody, peptide aptamer, and nucleic acid aptamer.

The terms “Fab” or “antigen-binding fragment” refers to two identical fragments of an antibody, typically prepared by enzymatic digestion that are confer binding specificity to an antibody. Papain digestion of antibodies produces two identical Fab fragment consisting of an entire light (L) chain along with the variable region domain of the heavy (H) chain (VH), and the first constant domain of one heavy chain (CH1). Pepsin treatment of an antibody yields a single large F(ab)2 fragment which roughly corresponds to two disulfide linked Fab fragments having divalent antigen-binding activity and is still capable of cross-linking antigen. Fab fragments differ from Fab′ fragments by having additional few residues at the carboxy terminus of the CH1 domain including one or more cysteines from the antibody hinge region.

The term “linker” refers to at least one atom that forms a covalent bond between two chemical entities. The term “linker” may refer to at least one atom that forms a covalent bond between the scaffold module and another covalent bond to the binding module. If the scaffold module and binding module is linked solely through peptide bonds, the linker is referred to as a “peptide linker”. Otherwise, the linker is referred to as a “chemical linker”. Further, a “flexible peptide linker” comprises mostly small, non-polar or polar amino acids whereas a “rigid peptide linker” comprises alpha-helix forming sequences and/or are rich in proline residues (Chen et al., 2013. Adv Drug Deliv Rev. 65(10):1357-1369).

The term “scaffold module” refers to a protein which comprises two antigen-binding sites and may act as a support structure for one or more binding modules. The binding modules may be attached to the scaffold module through a linker and/or the binding modules may be incorporated into any loop regions present in the scaffold module.

It is noted here that, as used in this specification and the appended claims, the singular forms “a,” “an,” and “the” include plural reference unless the context clearly dictates otherwise.

### T Cell Activation & Exhaustion

### T Cell Activation

The T cell receptor (TCR) on T cells binds to the antigen as presented by the MHC complex, on the surface of the antigen-presenting cell (APC). The binding of TCR/MHC/antigen triggers initial activation of the T cells. T cell signaling through the T cell antigen receptor (TCR)/CD3 complex triggers an array of signals that activate multiple effector pathways.

T cell activation is regulated, positively and negatively, by both TCR/CD3 complex generated signals and signals emanating from other cell surface receptors and/or delivered by soluble mediators to ensure that T cells respond to appropriate ligands and for the proper duration. A central tenet of T cell activation is that signaling solely through the TCR results in a state of anergy (a hyporesponsive state of T cells to a specific antigen that can be induced by a lack of costimulation).

Multiple surface receptors have been described as being capable of providing costimulation for T cells including CD28, CD30, CD5, CD2, ICOS, OX40 and 4-1BB (CD137). CD28 is expressed during the induction of an immune response and it promotes the expression of several other costimulatory molecules including ICOS, OX40 and CD137.

TCR-generated regulatory signals are also complemented by signals from ligation of other co-receptors such as cytotoxic T lymphocyte antigen-4 (CTLA-4) and programmed death-1 (PD-1 also known as CD279), both of which function to limit the expansion and activation of TCR-triggered T cells. In light of their function as negative regulators CTLA-4 and PD-1 are described as immune checkpoints. It is widely known that ligation of PD-1 expressed on activated T cells with PD-L1 ligand (also known as B7-H1 or CD274) activates a critical immune checkpoint leading to T cell tolerance, dysfunction and T cell exhaustion.

Long-term exposure to tumor antigen peptide/MHC class I complexes in the presence of inflammatory cytokines induces a distinct phenotype in T cells present in solid tumors, characterized by a progressive loss of effector functions, high and sustained expression of inhibitory immune checkpoint receptors, poor proliferative capacity, (Pauken K E and Wherry E J., Trends Immunol 2015; 36(4): 265-276 (2015), Wherry E J and Kurachi M., Nat Rev Immunol 2015 15(8): 486-499 (2015), metabolic dysregulation, poor memory re-call, and distinct transcriptional and epigenetic programs (McLane, L et al., Ann. Rev. Immunol. 37:457 (2019).

### T Cell Exhaustion

Dysfunctional or chronically stimulated T cells are a distinct lineage that develops upon repeated TCR stimulation in cancers and is found in mouse models and humans (Zajac A J et al, J. Exp. Med 188, 2205-2213(1998), Bakhli M Y Cytokine, 71, 339-347 (2011), Wherry and Kurachi Nat. Rev. Immunol 15, 486-499 (2015). Long-term exposure to tumor antigen peptide/MHC class I complexes in the presence of inflammatory cytokines induces a distinct phenotype in T cells present in solid tumors, characterized by a progressive loss of effector functions, high and sustained expression of inhibitory immune checkpoint receptors, poor proliferative capacity, (Pauken K E and Wherry E J., Trends Immunol 2015; 36(4): 265-276 (2015), Wherry E J and Kurachi M., Nat Rev Immunol 2015 15(8): 486-499 (2015), metabolic dysregulation, poor memory re-call, and distinct transcriptional and epigenetic programs (McLane, L et al., Ann. Rev. Immunol. 37:457 (2019).

Negative regulatory signals arising due to the activation of immunosuppressive checkpoints are thought to be the main mechanism for effector T-cell dysfunction in TME. The existence of tumor-antigen specific response and simultaneous progressive disease in cancer investigation identified PD-1 as a marker for exhaustion. Studies have shown that checkpoint blockade can at least partially reinvigorate T cell function (Fuertes Marraco S A, et al Front Immunol 6, 310(2015). Blockade of PD-1/PD-L1 interaction has been shown to restore T cell function partially.

### PD-1/PD-L1 Pathway

The PD-1/PD-L1 pathway has been studied extensively (Salmaninejad et al., 2019; Han et al., 2020; Makuku et al., 2021). PD-1 is a 55 kDa transmembrane protein with 288 amino acids containing an IgV-like extracellular domain followed by a transmembrane region and an intracellular tail with two phosphorylation sites for TCR signaling and regulation (Ishida, Agata, Shibahara, & Honjo). PD-L1 is a 33-kDa glycoprotein passing from the type 1 membrane with 290 amino acids with an Ig- and IgC-like domains in its extracellular region (Freeman et al., 2000). PD-1 and PD-L1 are immune checkpoint proteins.

PD-1 is expressed in a variety of activated immune cells, such as activated T cells, B cells and natural killer (NK), activated monocytes, dendritic cells (DCs), macrophages, and immature Langerhans cells. PD-1 is also upregulated on the surface of T cells constantly exposed to the antigen and is one of the markers of exhausted T cells (Ahmadzadeh et al., 2009). The transcription factors including NFAT, NOTCH, FOXO1 and IRF9 trigger transcription of PD-1 expression (Staron et al., 2014). Cytokines such as IL-2, IL-21, IL-15, IL-7, and type 1 IFNs can enhance PD-1 expression. IL-6 and IL-12, using a signal transducer and activator of transcription 3 (STAT3) and STAT4, respectively, increase PD-1 expression in spleen CD8 T cells (Salmaninejad et al., 2019).

PD-L1 is constitutively expressed by antigen-presenting cells (APCs) like macrophage, B cells, DCs and some epithelial cells, particularly under inflammatory conditions (Sharpe et al., 2007). The presence of PD-L1 in peripheral tissues is necessary to prevent autoimmune damage. In addition, PD-L1 is expressed by several types of tumor cells, such as non-small-cell lung cancer (NSCLC), hematologic malignancies and virus-infected cells, as an “adaptive immune mechanism” to escape anti-tumor responses (Ohaegbulam et al., 2015). Several transcription factors have been revealed to be involved in transcriptional upregulation of PD-L1 in cancer cells, such as hypoxia-inducible factor a (HIF-1a), STAT3 and NF-kB (Chen et al., 2015). Moreover, the cytokines produced by infiltrated immune cells such as IL-4, IL-10, TNFα, IFNγ and growth stem cell factor as well as bacterial LPS and VEGF upregulate the expression of PD-L1 gene (Ji et al., 2015).

The physiological role of PD-1 is the inhibition of functional T cells and the development of T regulatory (Treg) cells. Tregs and co-inhibitory immune checkpoints like PD-1/PD-L1 act as fail-safe to prevent aberrant and chronic activation of the immune system and immunoreactivity against self-antigens. However, it is well-known that the PD-1/PD-L1 pathway be co-opted by cancer cells or tumor-associated antigen-presenting cells as a means of evading anti-tumor T cell response and promoting tumor immune escape (Hargadon et al., 2018). Tumors can escape host immune surveillance by expressing PD-L1. Interaction between PD-1 and PD-L1 leads to downregulation of T cells and their apoptosis and exclusion from tumor microenvironment, thus causing cancer cells to escape from the immune response (Iwai et al., 2017).

### Targeting PD-1/PD-L1

PD-1 is a member of the B7/CD28 family of receptors that share a common structure: an immunoglobulin like extracellular domain, a transmembrane domain, and an intracellular domain containing the immunoreceptor tyrosine-based inhibitory and signaling motifs. When PD-1 is engaged by its ligands, PD-L1, such interaction leads to recruitment of the SRC homology phosphatases SHP1 and SHP2, which transmit the signal into the cell. PD-1 is predominantly expressed on activated T cells, the major cytotoxic effectors of the adaptive immune response, and the signal it transmits helps to damp down the T-cell mediated immune response. Studies have shown that blocking the inhibitory effects of PD-1 can elicit effective immune responses against tumor cells. PD-1/PD-L1 checkpoint inhibitors blocking this interaction increase immune cell proliferation and enhance the efficacy of the body's natural antitumor surveillance system. Understanding the mechanism of evasion of cancer to immune checkpoints is thus crucial to the approach of personalizing the delivery of immunotherapy.

Two separate signals are required to fully activate T cells that keep cytotoxic activity in check. The first comes from the interaction between the T-cell receptor and major histocompatibility complex-presented antigen epitopes on the surface of an antigen-presenting cell (APC). The second comes from engagement of costimulatory receptor-ligand pairs on the T-cell and APC surfaces. PD-1 belongs to a group of coinhibitory receptors that regulate T-cell activity via ligand binding, which can drive T cells into a state known as exhaustion, in which they are unable to proliferate or perform their effector functions.

Blockade of the PD-1/PD-L1 pathway has been shown to reinvigorate/restore the function of exhausted tumor-infiltrating T lymphocytes. For example, treatment with anti-PD-1/PD-L1 and anti-CTLA-4 immune checkpoint inhibitors has been reported to reinvigorate dysfunctional TILs and augment their anti-tumor effects (Wherry and Kurachi, 2015; Zarour, 2016; Miller et al., 2019).

### CD137 Co-Stimulatory Pathway

CD137, which is also called as 4-1BB, or TNFRSF9 (TNF Receptor Superfamily Member 9) was first identified as an inducible co-stimulatory receptor expressed on activated T cells, is a 30 kDa membrane-spanning glycoprotein of the tumor necrosis factor (TNF) receptor superfamily (TNFRSF). Current understanding of 4-1BB indicates that expression is generally activation dependent and encompasses a broad sub-set of immune cells including activated T cells, activated natural killer (NK) and natural killer T (NKT) cells, regulatory T cells, dendritic cells (DC) including follicular DC, stimulated mast cells, differentiating myeloid cells, monocytes, neutrophils, and eosinophils. 4-4BB expression has also been demonstrated on tumor vasculature and atherosclerotic endothelium. The ligand that stimulates CD137 (CD137L) is expressed on activated antigen-presenting cells (APCs), myeloid progenitor cells, and hematopoietic stem cells (Wang et al., Immunological Reviews, 2009, 229, 192-215).

CD137 is undetectable on the surface of naive T cells. Upon stimulation TCR signaling, CD137 expression induces and peaks at 2-3 days post stimulation and declines after 3 days. CD137 expresses on the cell surface of activated T cells as both monomers and dimers. Based on homology to other members in the family of TNFRSF, ligand binding induces receptor trimerization resulting in activation of the receptor. Some members of the TNFRSF can exist in a soluble form following cleavage of the extracellular domain from the cell surface. Soluble 4-1BB and soluble 4-1BBL have been detected in the serum of some patients with autoimmune diseases and cancers (Wang et al., Immunological Reviews, 2009, 229, 192-215).

CD137 is a member of the TNF-receptor (TNFR) superfamily without known intrinsic enzymatic activity in its cytoplasmic domain. It relies on the TNFR-Associated-Factor (TRAF) family of adaptor proteins to build the CD137 signalosome for transducing signals into the cell. Upon CD137 activation by binding of CD137L trimers or by crosslinking with agonist monoclonal antibodies, TRAF1, TRAF2, and TRAF3 are readily recruited to the cytoplasmic domain of CD137, likely as homo- and/or heterotrimers with different configurations, initiating the construction of the CD137 signalosome, which results in downstream activation of NF-κB and the mitogen-activated protein (MAP) kinase cascade including ERK, JNK, and p38 MAP kinases (Bartkowiak et al. Clin. Cancer Res. 2018, 24, 1138-1151).

CD137 plays a critical role in sustaining effective T cell immune responses and in generating immunological memory. The expression profile of CD137, as well as its unique ability to potentiate robust effector responses in multiple subsets of lymphocytes relevant for tumor immunity, makes CD137 a uniquely appealing target for immunotherapy.

Multiple studies of mouse and human T cells indicate that CD137 promotes cellular proliferation, survival, and cytokine production. CD137 agonist antibodies have been shown to markedly enhance cytolytic T lymphocyte responses. Agonist CD137 antibodies as monotherapy or in combination with other therapies, such as checkpoint inhibitors, have provided evidence of anti-tumor benefit in prophylactic and therapeutic settings. Stimulation of CD137 has been shown to result in durable anti-tumor protective T-cell memory responses in vivo (Fisher et al, Cancer Immunol Immunother 2012, 61, 1721-1733). More recently, CD137 agonist antibodies have been shown to increase expression of the cellular adhesion molecules ICAM-1, VCAM-1, and E-selectin on tumor vasculature resulting in increased T-cell migration into tumor microenvironment (Palazon et al, Cancer Research, 2011, 71(3), 8001-811).

However, the puzzling observation that both CD137−/− mice and agnostic CD137 antibodies exhibit enhanced antitumor activity indicates that the mere activation of CD137 signaling cannot fully explain its antitumor effect. Multiple studies have reported CD137 signaling upon binding to CD137L. Recently, accumulating evidence showing that CD137L/CD137 interaction results in bi-directional signaling. Since CD137L is mainly expressed on activated APCs such as DCs, reverse signaling from CD137L have been reported to affect the development and functions of DCs (Kwon, Immune Network, 2015, 15(3), 121-124). In 2017, report findings demonstrated reverse signaling by CD137 ligand (CD137L) in antigen-presenting dendritic cells (DCs) in tumors that explained these paradoxical results. Specifically, CD137L reverse signaling suppressed intra-tumoral differentiation of IL12-producing CD103+ DC and type 1 tumor-associated macrophages (TAMs), which play an important role to generate IFNγ-producing CD8+ cytotoxic T lymphocytes. Notably, CD137L blockade increased levels of IL12 and IFNγ, which further promoted intra-tumoral differentiation of IFNγ-producing CD8+ T cells, IL12-producing CD103+ DC, and type 1 TAM within tumors. Therefore, activating CD137 signaling in T cells while blocking CD137L reverse signaling in DCs should fully elicit the anti-tumor activity of C137 pathway (Kang et al., Cancer Research, 2017, 77 (21), 5989-6000).

High doses of CD137 agonist antibodies in naive and tumor-bearing mice have been reported to induce T-cell infiltration to the liver and elevations of aspartate amino-transferase (AST) and alanine aminotransferase (ALT), an indication of liver inflammation or damage. Based on clinical studies evaluating the efficacy of an anti-CD137 antibody (urelumab, Bristol Myers), activation of CD137 activity can elicit therapeutic anti-tumor activities. However, liver toxicity limits its clinical development. Clinical evaluation of a second anti-CD137 (utomilumab, Pfizer) demonstrated an acceptable safety profile but limited clinical efficacy. To date there are no approved therapeutic antibodies directed against CD137.

### Bispecifics Targeting PD/PD-L1 Checkpoint and CD137

As co-stimulatory receptors play key roles in regulating the effector functions of T cells, agonism of a costimulatory pathway could improve checkpoint inhibition efficacy, and may lead to durable antitumor responses. A bispecific molecule designed to target the PD-1/PD-L1 pathway and a T cell co-stimulatory molecule may dis-inhibit the checkpoint and at the same time co-stimulate T cells to provide efficient induction of anti-tumor immunity. Depending on aspects of the molecular design, T cell activation could occur through the trans binding and be dependent upon PD-L1 binding, thereby effectively limiting the immune activity to the tumor microenvironment. Suitable costimulatory targets on T cells include, but are not limited to CD137, OX40, CD28, CD27, CD226, GITR, ICOS, TNFRSF25, LIGHT (TNFSF14), TIM-1, and LFA-1.

Previous preclinical experiments and clinical data clearly support that agonizing a co-stimulatory pathway can be a potently effective strategy for re-invigorating T-cell responses in cancers, particularly when used in combination with other immune-activating strategies. Binding proteins that are bispecific for PD-L1 and a co-stimulatory molecule on T cells should mediate simultaneous binding to PD-L1-expressing antigen-presenting cells (APCs) or tumor cells and activated T cells, resulting in conditional activation of tumor-specific T cells such as tumor infiltrating T cells and CD8⁺ cytolytic T cells, to alleviate on-target off tumor toxicity of agonist anti-CD137 antibody.

### TGFβ

The TGFβ super-family is a large group of structurally associated proteins including TGFβ, nodal, activin, lefty, bone morphogenetic proteins and growth and differentiation factor. TGFβ signaling is transduced through Smad and non-Smad pathways. TGFβ is a pleiotropic cytokine with a crucial function in mediating immune suppression and evasion of immunosurveillance in the TME. TGFβ is aberrantly produced by tumors, and promotes cancer progression primarily by suppressing both the innate and adaptive immune systems. As a negative regulator of anti-tumor immunity, TGF-β impairs the efficacy of anti-PD-1/PD-L1 and induces drug resistance.

It has been reported that only about 10 to 30% of patients with most carcinomas achieve objective responses when treated with anti-PD-1/PD-L1 monotherapies, even in trials that selectively enroll only patients whose pre-treatment tumor specimens express PD-L1 (Lipson, E J et al, Semin. Oncol. 42(4):587 (2015). One possible explanation for this lack of response is the immunosuppressive nature of the tumor microenvironment attributed to the presence of immunosuppressive cells (e.g., regulatory T cells (Tregs) and other factors (e.g., cytokines and metabolic pathways) that function to inhibit T cell priming or suppress effector T cell function. The effect of anti-PD-1/PD-L1 therapy has been reported to be limited in TMEs characterized by hyperactive TGFβ signaling (Tauriello D V F, et al, Nature, 554:538-543 (2018).

The effects of TGFβ signaling are mediated by three TGFβ ligands (TGF-β1-3), all of which exist as homodimers, through TGFβ type 1 (TGFβR1) and type 2 receptors TGFβR2. There are numerous cellular context-dependent factors tightly implicated in the balance of TGFβ signaling (Liu S, et al., Mol Med Rep 17: 699-704 (2018) in the tumor microenvironment. TGFβ has been shown to be an important factor for suppressing antitumor immune responses, but the precise role of T-cell and tumor-derived TGF-β remains poorly understood.

Tregs, monocytic myeloid-derived suppressor cells (MDSC)s, alternatively polarized macrophages (M2 phenotype), and their associated soluble factors are well-recognized inhibitory mechanisms that can suppress anti-tumor immunity. MDSCs have been reported to be the primary source of TGFβ in the tumor microenvironment.

Tregs are commonly found in solid tumors and can promote immunosuppression by several mechanisms, including competing for activating cytokines with effector cells and the secretion of immunosuppressive cytokines. For example, Tregs contribute to the level of transforming growth factor-beta (TGFβ) in the tumor microenvironment, which is an immunosuppressive factor that subverts both adaptive immune priming and effector responses.

Designing therapeutic agents that target tumor microenvironment modulators is a recognized strategy used to optimize tumor immunotherapy. For example, the interaction of TGFβ and immune cells has been identified as an important regulatory axis in the tumor microenvironment and cancer progression. TGFβ regulates the function of numerous immune cells by inducing the differentiation of Tregs, reducing the cytotoxicity of T cells and natural killer (NK) cells, restricting the tumoral infiltration of immune cells, and suppressing antigen presentation by dendritic cells (DCs) (Yi, M et al. J. Hematol. Oncol. 14(1):27 (2021). The main strategy for inhibition of the TGF-β signaling pathway is to design therapeutic agents that interfere with the binding of TGF-β to its receptors, that block intracellular signaling, or disrupt expression using antisense oligonucleotides.

### TGFβ as Tumor Microenvironment Modulator

The effect of anti-PD-1/PD-L1 immunotherapy is limited in TMEs characterized by hyperactive TGFβ signaling. Data from preclinical studies support the premise that blocking or antagonizing TGFβ can potentially disrupt several factors contributing to the immunosuppressive effects of TGFβ. A study by Budhu et al. found that the antitumor activity of T cells was inhibited by tumor-resident regulatory T (Treg) cells, and that the immunosuppression is dependent on the presence of TGFβ on the Treg cell surface. A blocking antibody against TGF-β reversed the immunosuppression and boosted the antitumor response (S. Budhu et al, Sci. Signal. 10, eaak9702 (2017). Other published data suggest that TGFβ restrains the antitumor immune response by blocking T cells from infiltrating the tumor. Studies performed in a mouse model of urothelial cancer showed that combined therapy with antibodies targeting TGF-β signaling and PD-L1 increase the ability of T cells to penetrate tumors, induced tumor regression and enhanced the antitumor immune response (Mariathasan, S et al, Nature 554, 544-548 (2018).

Tauriello, D. V. F. et al. studied mice expressing the four mutations associated with colorectal cancer and found that metastases in the mice was characterized by reduced T cell infiltration and active TGFβ in the stroma. Inhibition of the PD-1-PD-L1 immune checkpoint provoked a limited response in the model system, however inhibition of TGFβ unleashed a potent and enduring cytotoxic T-cell response that prevented metastasis. In mice with progressive liver metastatic disease, blocking of TGFβ signaling rendered the tumors susceptible to anti-PD-1/PD-L1 therapy and led to increased survival (Tauriello, Nature 554, 544-548 (2018).

M7824 (bintrafusp alfa) is a bifunctional fusion protein under development by Merck KGaA, Darmstadt, Germany, and GlaxoSmithKline. M7824 comprises VH and VL sequences derived from the humanized IgG1 monoclonal antibody avelumab genetically fused via a flexible (Gly₄Ser)₄Gly linker to the N terminus of the soluble extracellular domain of TGF-βRII (136 amino acids). It functions as a cytokine trap for all three TGF-β (TGF-β1-3) ligand isoforms.

Several preclinical studies have shown bintrafusp alfa capable of (1) preventing or reverting TGF-β-induced epithelial-mesenchymal transition in human carcinoma cells; this alteration in tumor cell plasticity was shown to render human tumor cells more susceptible to immune-mediated attack as well as to several chemotherapeutic agents; (2) altering the phenotype of natural killer and T cells, thus enhancing their cytolytic ability against tumor cells; (3) mediating enhanced lysis of human tumor cells via the antibody-dependent cell-mediated cytotoxicity mechanism; (4) reducing the suppressive activity of Treg cells; (5) mediating antitumor activity in numerous preclinical models and (6) enhancing antitumor activity in combination with radiation, chemotherapy and several other immunotherapeutic agents designed to simultaneously block the PD-L1 and the TGF beta pathways (Lind H, et al, J Immunother Cancer, February; 8(1):e000433(2020). Treatment with the anti-PD-L1/TGFβ Trap (bintrafusp) has been shown to elicit a synergistic anti-tumor effect reported to be superior to that of single agent anti-PD-L1 or TGFβ trap protein (U.S. Pat. No. 9,676,863).

The observed synergy is attributed to the simultaneous blockade of the interaction between PD-1 on immune cells and PD-L1 on tumor cells and the neutralization of TGFβ in the tumor microenvironment (U.S. Pat. No. 9,676,863). The synergistic effect is presumed to be based on the simultaneous blocking of the two major immune escape mechanisms. The depletion of TGFβ is achieved by antagonizing the TGFβ cytokine levels in the tumor microenvironment (as a consequence of the anti-PD-L1 targeting of tumor cells) and the destruction of TGFβ through PD-L1 receptor-mediated endocytosis (U.S. Pat. No. 9,676,863).

### PD-L1 Binding Protein Targeting PD/PD-L1 Checkpoint and a TGFβ

It has been reported that the dual blockade of PD-1/PD-L1 and TGFβ has a synergistic anti-tumor activity (Chen, X et al. Int. J. Cancer 143:2561 (2018) and Yi, M et al. J. Hematol. Oncol. 14(1):27 (2021). Considering that the immunosuppressive effects of the PD-1/PD-L1 axis and TGFβ are complementary and independent, it is rational to design PD-L1 binding proteins that are capable of blocking TGFβ signaling to enhance the efficacy of anti-PD-L1 immunotherapy (Yi, M et al. J. Hematol. Oncol. 14(1):27 (2021)). In addition, bi- or tri-specific binding proteins that bind PD-L1 and comprise a TGF-β pathway antagonist may provide an effective option for patients resistant to PD-1/PD-L1 monotherapies (Kim et al., J Hematol Oncol, 14:55 (2021)).

To enhance the efficacy of anti-PD-L1 immunotherapy, binding proteins which can simultaneously block the PD-1/PD-L1 axis and the TGFβ signaling pathway are provided. In some embodiments, the binding proteins can also bind CD137 and provide a costimulatory signal promoting T cell responsiveness.

### PD-L1 Monospecifics

In some embodiments, the disclosed PD-L1 monospecifics (1923Ab2 and 1923Ab3) are specific for (e.g., specifically bind) human PD-L1. These binding proteins and fragments thereof are characterized by unique sets of CDR sequences, specificity for PD-L1 and exhibit potent inhibitory activities of the PD-1/PD-L1 signaling. More specifically, in one aspect the disclosure relates to binding proteins that bind human PD-L1, and to their use as monotherapies or in combination with other anti-cancer agents to modulate the PD-L1-mediated activity of cells localized to the tumor microenvironment. In an alternative embodiment, the disclosed binding proteins that bind PD-L1 can also be used as subunits of bispecifics and trispecifics designed to dis-inhibit/release effector T cells from PD-1/PD-L1 checkpoint inhibition.

In an alternative aspect the disclosure relates to the use of the disclosed binding proteins that bind PD-L1 to design bispecifics or trispecifics that bind PD-L1 and their use to disinhibit the PD-L1/PD-L1 checkpoint and promote T cell activation.

In an exemplary embodiment, the invention provides a binding protein that binds PD-L1, comprising an antibody scaffold module comprising: (a) a heavy chain variable region sequence comprising the CDR sequences for 1923Ab2 in TABLE 1; and a light chain variable region sequence comprising the CDR sequences for 1923Ab2 in TABLE 2.

In an exemplary embodiment, the invention provides a binding protein that binds PD-L1, comprising an antibody scaffold module comprising: (a) a heavy chain variable region sequence comprising the CDR sequences for 1923Ab3 in TABLE 1; and a light chain variable region sequence comprising the CDR sequences for 1923Ab3 in TABLE 2.

TABLE 1
CDR Sequences of PD-L1 Binding Heavy Chain Variable Regions

| PD-L1 | | | |
|---|---|---|---|
| Monospecific | | | |
| Ref # | CDR1 | CDR2 | CDR3 |
| 1923Ab2 | SEQ ID NO: 5 | SEQ ID NO: 6 | SEQ ID NO: 7 |
| 1923Ab3 | SEQ ID NO: 11 | SEQ ID NO: 12 | SEQ ID NO: 13 |

TABLE 2
CDR Sequences of PD-L1 Binding Light Chain Variable Regions

| PD-L1 | | | |
|---|---|---|---|
| Monospecific | | | |
| Ref # | CDR1 | CDR2 | CDR3 |
| 1923Ab2 | SEQ ID NO: 8 | SEQ ID NO: 9 | SEQ ID NO: 10 |
| 1923Ab3 | SEQ ID NO: 14 | SEQ ID NO: 9 | SEQ ID NO: 15 |

In some embodiments, the binding proteins that bind PD-L1 can be monoclonal, chimeric, bispecific or trispecific with a heavy chain region sequence that comprises VH (SEQ ID NOs. 1 and 3, for example) and VL (SEQ ID NOs. 2 and 4, for example), and the constant region may be IgG1, IgG2, IgG3 or IgG4. In a further embodiment, the antibody of the present invention is a human IgG1-type with Fc silencing mutations (L234A L235A or N297A).

In some embodiment, it is advantageous that the disclosed anti-PD-L1 antibodies are Fc-engineered.

In a still further embodiment, the invention provides for binding proteins that bind PD-L1 including a heavy chain sequence and a light chain sequence, wherein the heavy chain sequence has at least 85% sequence identity to SEQ ID NOs. 42 or 45 and the light chain sequence has at least 85% sequence identity to SEQ ID NO. 40.

In one embodiment, the disclosed antibodies bind to human or cynomolgus monkey PD-L1 and is capable of blocking the interaction between human PD-L1 and PD1 receptor.

In one embodiment, the binding protein binds human PD-L1 with a KD of 5×10⁻⁹ M or less, preferably with a KD of 2×10⁻⁹ M or less, and even more preferably with a KD of 1×10⁻⁹ M or less.

In a further embodiment, the invention is related to binding proteins that bind human PD-L1, or a fragment thereof, which cross-compete for binding to PD-L1 with an antibody (Tecentriq) according to the invention as described herein.

In some embodiments, binding proteins that bind PD-L1 or fragments thereof exhibit one or more of the following structural and functional characteristics, alone or in combination: (a) is specific for human PD-L1, (b) cross-reacts with cynomolgus PD-L1, (c) disrupts the binding of PD-L1 to PD-1, or (d) removes T cell PD-L1 mediated checkpoint inhibitory signal.

Disruption of PD-L1/PD-1 interaction and dis-inhibition of the activation checkpoint by the disclosed binding proteins that bind PD-L1 was investigated using multiple in vitro assays. The bioactivity of the anti-PD-L1 moiety was determined by its ability to disrupt interaction between PD-1 and PD-L1 to restore TCR signaling using PD-1/PD-L1 blockage assay.

### CD137 Monospecifics

In some embodiments, the disclosed CD137 monospecifics (1923Ab4, 1923Ab5 and 1923Ab6) are specific for (e.g., specifically bind) human CD137. These binding proteins and fragments thereof are characterized by unique sets of CDR sequences, specificity for CD137 and are useful in cancer immunotherapy as monotherapy or in combination with other anti-cancer agents. As demonstrated herein the binding proteins that bind CD137 can also be used as subunits of bispecifics and trispecifics designed to reinvigorate effector T cells released from PD1/PD-L1 checkpoint inhibition. More specifically, the disclosure relates to binding proteins that bind human CD137, and to their use to modulate the CD137-mediated activity of cells localized to the tumor microenvironment.

In an exemplary embodiment, the invention provides a binding protein that binds CD137, comprising an antibody scaffold module comprising: (a) a heavy chain variable region sequence comprising the CDR sequences for 1923Ab4 in TABLE 3; and a light chain variable region sequence comprising the CDR sequences for 1923Ab4 in TABLE 4.

In an exemplary embodiment, the invention provides a binding protein that binds CD137, comprising an antibody scaffold module comprising: (a) a heavy chain variable region sequence comprising the CDR sequences for 1923Ab5 in TABLE 3; and a light chain variable region sequence comprising the CDR sequences for 1923Ab5 in TABLE 4.

In an exemplary embodiment, the invention provides a binding protein that binds CD137, comprising an antibody scaffold module comprising: (a) a heavy chain variable region sequence comprising the CDR sequences for 1923Ab6 in TABLE 3; and a light chain variable region sequence comprising the CDR sequences for 1923Ab6 in TABLE 4.

TABLE 3
CDR Sequences of CD137 Binding Heavy Chain Variable Regions

| CD137 | | | |
|---|---|---|---|
| Monospecific | | | |
| Ref # | CDR1 | CDR2 | CDR3 |
| 1923Ab4 | SEQ ID NO: 5 | SEQ ID NO: 22 | SEQ ID NO: 23 |
| 1923Ab5 | SEQ ID NO: 27 | SEQ ID NO: 28 | SEQ ID NO: 29 |
| 1923Ab6 | SEQ ID NO: 32 | SEQ ID NO: 33 | SEQ ID NO: 34 |

TABLE 4
CDR Sequences of CD137 Binding Light Chain Variable Regions

| CD137 | | | |
|---|---|---|---|
| Monospecific | | | |
| Ref # | CDR1 | CDR2 | CDR3 |
| 1923Ab4 | SEQ ID NO: 24 | SEQ ID NO: 25 | SEQ ID NO: 26 |
| 1923Ab5 | SEQ ID NO: 30 | SEQ ID NO: 9 | SEQ ID NO: 31 |
| 1923Ab6 | SEQ ID NO: 35 | SEQ ID NO: 36 | SEQ ID NO: 37 |

In some embodiments, the binding proteins that bind CD137 can be monoclonal, chimeric, bispecific or trispecific with a heavy chain region sequence that comprises VH (SEQ ID NOs. 16, 18, and 20, for example) and VL (SEQ ID NOs. 17, 19, and 21, for example), and the constant region may be IgG1, IgG2, IgG3 or IgG4. In a further embodiment, the antibody of the present invention is a human IgG1-type with Fc silencing mutations (L234A L235A or N297A).

In some embodiment, it is advantageous that the disclosed anti-CD137 antibodies are Fc-engineered.

In a still further embodiment, the invention provides for binding proteins that bind CD137 including a heavy chain sequence and a light chain sequence, wherein the heavy chain sequence has at least 85% sequence identity to the heavy chain sequence: SEQ ID NO. 75 and the light chain sequence has at least 85% sequence to the light chain sequence: SEQ ID NO. 76.

Applicants sought to discover binding proteins that bind CD137 that demonstrate the desired profile to overcome on-target off-tumor toxicity and immunosuppressive setting for better immunotherapy. The disclosed binding proteins that bind CD137 may be particularly beneficial for tumor microenvironments enriched in exhausted T cells or regulatory T cells that contribute to anti-PD-1/PD-L1 resistance.

In some embodiments, binding proteins that bind CD137 and fragments thereof exhibit one or more of the following structural and functional characteristics, alone or in combination:
(a) is specific for human CD137,
(b) cross-reacts with cynomolgus CD137,
(c) disrupts (e.g., reduces or prevents) human CD137L binding to CD137,
(d) exhibits fast on and fast off properties to CD137,
(e) possess crosslinking dependent agonistic activity to CD137 signaling, or
(f) activates T cells in crosslinking dependent manner.

In one embodiment, the disclosed binding proteins activate CD137 signaling in the presence of crosslinker. In T cell activation assay using primary PBMCs, they enhanced anti-CD3 stimulated IFN-gamma release in a crosslinking dependent manner.

In some embodiments, it is advantageous that the disclosed binding proteins bind both to human CD137 and to cynomolgus CD137. Cross-reactivity with CD137 expressed on cells in cynomolgus monkey (e.g. Macaca fascicularis), is advantageous because it enables animal testing of the antibody molecule without having to use a surrogate antibody. The disclosed binding proteins that bind CD137, 1923Ab4, 1923Ab5 and 1923Ab6, all bind CD137 from cynomolgus monkey with notable affinity. Although the disclosed antibodies do not bind mouse CD137, humanized CD137 mice are available and can be used to study efficacy of the disclosed antibodies in preclinical mouse tumor model in vivo.

T cell activation by the disclosed anti-CD137 antibodies was investigated using multiple in vitro assays. The bioactivity of the anti-CD137 moiety was determined by its ability to induce crosslinking dependent CD137 signaling using assay cells overexpressing CD137 and carrying NFκB luciferase reporter. In addition, crosslinked anti-CD137 enhances anti-CD3 stimulated IFNγ release in the PBMCs.

In some embodiments, the binding proteins disclosed herein may comprise one or more conservative amino acid substitutions. A person of skill in the art will recognize that a conservative amino acid substitution is a substitution of one amino acid with another amino acid that has similar structural or chemical properties, such as, for example, a similar side chain. Exemplary conservative substitutions are described in the art, for example, in Watson et al., Molecular Biology of the Gene, The Benjamin/Cummings Publication Company, 4th Ed. (1987).

“Conservative modifications” refer to amino acid modifications that do not significantly affect or alter the binding characteristics of the binding protein containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al. (1998) Acta Physiol Scand Suppl 643: 55-67; Sasaki et al. (1998) Adv Biophys 35: 1-24). Amino acid substitutions to the binding proteins disclosed herein may be made by known methods for example by PCR mutagenesis (U.S. Pat. No. 4,683,195).

In some embodiments, the binding protein comprises a variable heavy chain sequence that comprises an amino acid sequence with at least about 95%, about 96%, about 97%, about 98%, or about 99%, sequence identity to the amino acid sequence set forth in SEQ ID NOs: 16, 18, or 20. In other embodiments, the binding protein retains the binding and/or functional activity of a binding protein that comprises the variable heavy chain sequence of SEQ ID Nos: 16, 18, or 20. In still further embodiments, the binding protein comprises the variable heavy chain sequence of SEQ ID Nos: 16, 18, or 20 and have one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NOs: 16, 18, or 20 (based on the numbering system of Kabat).

In particular embodiments, the binding protein comprises a variable heavy chain sequence with at least about 95%, about 96%, about 97%, about 98%, or about 99% sequence identity to the binding protein heavy chain variable region sequence set forth in SEQ ID NOs: 16, 18, or 20, comprises one or more conservative amino acid substitutions in a framework region (based on the numbering system of Kabat), and retains the binding and/or functional activity of a binding protein that comprises a variable heavy chain sequence as set forth in SEQ ID NOs: 16, 18, or 20 and a variable light chain sequence as set forth in SEQ ID NOs: 17, 19, or 21.

In some embodiments, the binding protein comprises a variable light chain sequence that comprises an amino acid sequence with at least about 95%, about 96%, about 97%, about 98%, or about 99%, sequence identity to the amino acid sequence set forth in SEQ ID NOs: 17, 19, or 21. In other embodiments, the binding protein retains the binding and/or functional activity of a binding protein that comprises the variable light chain sequence of SEQ ID Nos: 17, 19, or 21. In still further embodiments, the binding protein comprises the variable light chain sequence of SEQ ID Nos: 17, 19, or 21 and have one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NOs: 17, 19, or 21 (based on the numbering system of Kabat).

In particular embodiments, the binding protein comprises a variable light chain sequence with at least about 95%, about 96%, about 97%, about 98%, or about 99% sequence identity to the binding protein light chain variable region sequence set forth in SEQ ID NOs: 17, 19, or 21, comprises one or more conservative amino acid substitutions in a framework region (based on the numbering system of Kabat), and retains the binding and/or functional activity of a binding protein that comprises a variable heavy chain sequence as set forth in SEQ ID NOs: 16, 18, or 29 and a variable light chain sequence as set forth in SEQ ID NOs: 17, 19, or 21.

### PD-L1/CD137 Bispecifics

In certain embodiments, the disclosure provides PD-L1/CD137 bispecifics comprising a PD-L1 antibody scaffold module derived from an anti-PD-L1 antibody and a CD137 first binding module derived from an anti-CD137 antibody, wherein the bispecific agonizes CD137 and activates T cells in a PD-L1-dependent manner.

In some embodiments, the PD-L1/CD137 bispecific comprise a heavy chain disclosed in TABLE 5. For example, the PD-L1/CD137 bispecific may comprise an HC with a set of CDR sequences derived from the VH of 1923Ab3, and a modified human IgG1 Fc region with or without amino acid modifications to diminish or ablate Fc receptor functions. In some embodiments the HC may further comprise an scFv with a set of CDR sequences derived from the VH and VL regions of a binding protein that binds CD137 disclosed herein, for example 1923Ab4. In some embodiments the scFv may be attached to the HC by a linker positioned at the N- or C-terminus of the HC.

In some embodiments, the PD-L1/CD137 bispecific comprise a light chain disclosed in TABLE 5. For example, the PD-L1/CD137 bispecific may comprise an LC with a set of CDR sequences derived from the VL of 1923Ab3. In an embodiment the LC may further comprise an scFv with a set of CDR sequences derived from the VH and VL regions of a binding protein that binds CD137 disclosed herein, for example 1923Ab4. In some embodiments, the scFv may be attached to the LC by a linker positioned at the N- or C-terminus of the LC.

TABLE 5
PD-L1/CD137 Bispecifics

| Bispecific Ref # | Heavy chain (HC) | Light chain (LC) |
|---|---|---|
| 1923Ab8 | SEQ ID NO: 38 | SEQ ID NO: 40 |
| 1923Ab11 | SEQ ID NO: 44 | SEQ ID NO: 40 |
| 1923Ab12 | SEQ ID NO: 45 | SEQ ID NO: 46 |
| 1923Ab13 | SEQ ID NO: 47 | SEQ ID NO: 40 |
| 1923Ab18 | SEQ ID NO: 50 | SEQ ID NO: 40 |

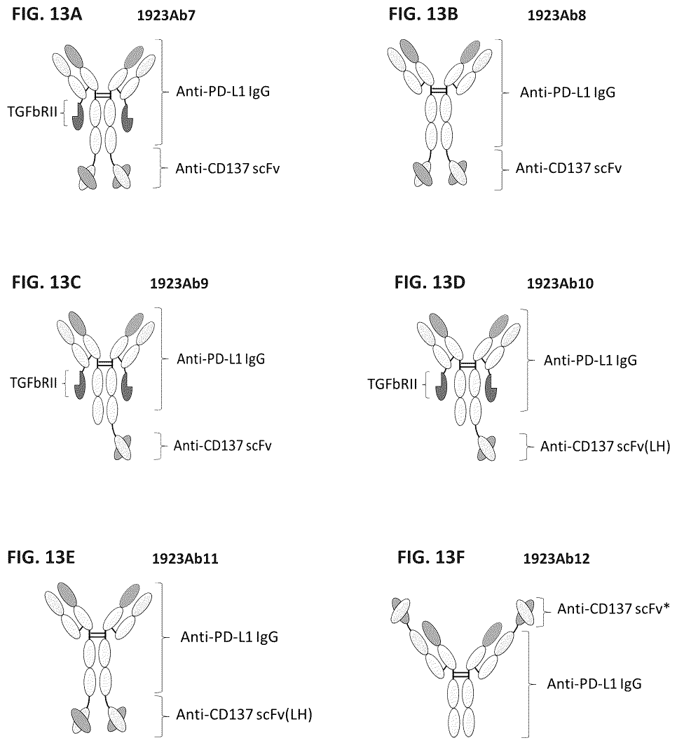

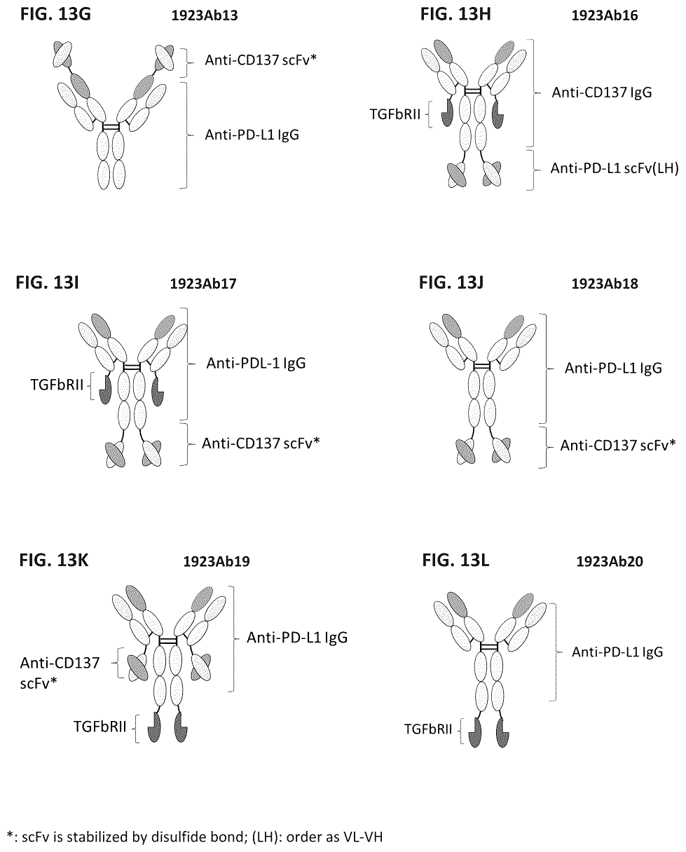

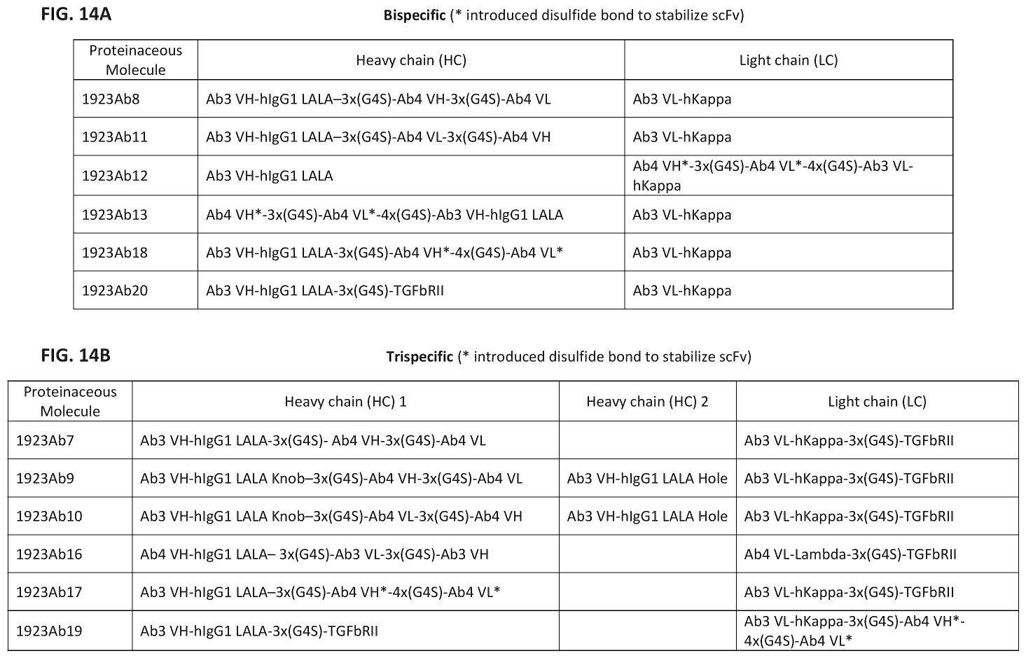

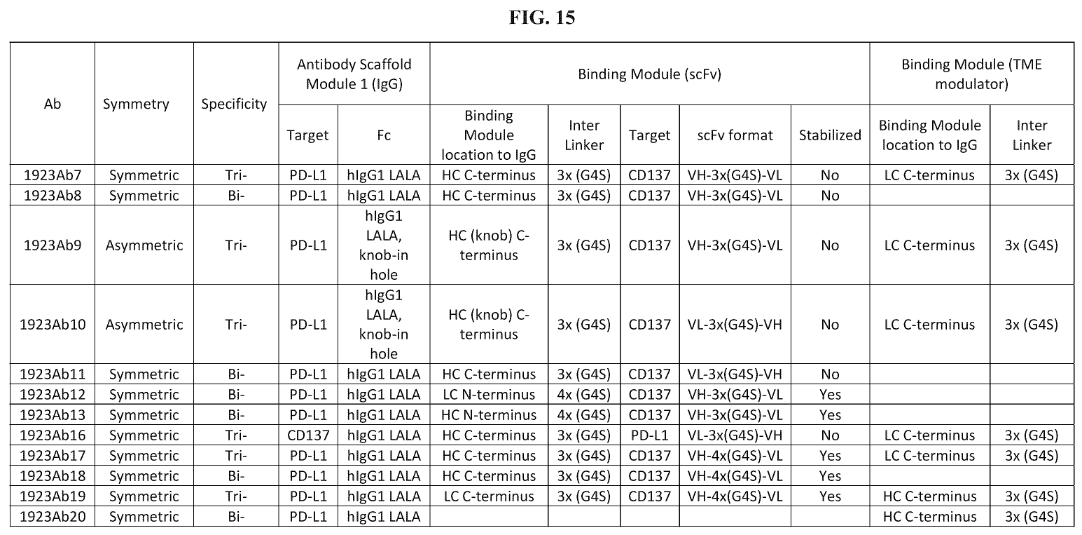

In some embodiments, the PD-L1/CD137 bispecific 1923Ab8 (FIG. 13B) contains two Fabs from 1923Ab3 that bind PD-L1, human IgG1 Fc with L234A L235A mutations, and two scFv fragments (VH precedes VL) derived from 1923Ab4 attached to the C-terminus of the Ab3 heavy chains. FIG. 14(A) provides a description of the heavy chain and light chains of 1923Ab8. FIG. 15 provides a more detailed description of the subcomponents of the disclosed binding proteins. The amino acid sequences of the heavy and light chains are provided in SEQ ID NO: 38 and SEQ ID NO: 40, respectively.

In some embodiments, the PD-L1/CD137 bispecific 1923Ab11 (FIG. 13E) contains two Fabs from 1923Ab3 that bind PD-L1, human IgG1 Fc with L234A L235A mutations, and two scFv fragments (VL precedes VH) derived from 1923Ab4 attaching to the C-terminus of the Ab3 heavy chains. FIG. 14 (A) provides a description of the heavy chain and light chains of 1923Ab11. FIG. 15 provides a more detailed description of the subcomponents. The amino acid sequences of the heavy and light chains are provided in SEQ ID NO: 44 and SEQ ID NO: 40, respectively.

In some embodiments, the PD-L1/CD137 bispecific 1923Ab12 (FIG. 13F) contains two Fabs from 1923Ab3 that bind PD-L1, human IgG1 Fc with L234A L235A mutations, and two disulfide bond-stabilized scFv fragments (VH precedes VL) derived from 1923Ab4 attaching to the N-terminus of the Ab3 light chains. FIG. 14(A) provides a description of the heavy chain and light chains of 1923Ab12. FIG. 15 provides a more detailed description of the subcomponents. The amino acid sequences of the heavy and light chains are provided in SEQ ID NO: 45 and SEQ ID NO: 46, respectively.

In some embodiments, the PD-L1/CD137 bispecific 1923Ab13 (FIG. 13G) contains two Fabs from 1923Ab3 that bind PD-L1, human IgG1 Fc with L234A L235A mutations, and two disulfide bond-stabilized scFv fragments (VH precedes VL) derived from 1923Ab4 attaching to the N-terminus of the Ab3 heavy chains. FIG. 14(A) provides a description of the heavy chain and light chains of 1923Ab13. FIG. 15 provides a more detailed description of the subcomponents. The amino acid sequences of the heavy and light chains are provided in SEQ ID NO: 47 and SEQ ID NO: 40, respectively.

In some embodiments, the PD-L1/CD137 bispecific 1923Ab18 (FIG. 13J) contains two Fabs from 1923Ab3 that bind PD-L1, human IgG1 Fc with L234A L235A mutations, and two disulfide bond-stabilized scFv fragments (VH precedes VL) derived from 1923Ab4 attaching to the C-terminus of the Ab3 heavy chains. FIG. 14(A) provides a description of the heavy chain and light chains of 1923Ab18. FIG. 15 provides a more detailed description of the subcomponents. The amino acid sequences of the heavy and light chains are provided in SEQ ID NO: 50 and SEQ ID NO: 40, respectively.

The bioactivity of the anti-PD-L1 moiety was determined by its ability to disrupt interaction between PD-1 and PD-L1 to restore TCR signaling using PD-1/PD-L1 blockage assay. The bioactivity of the anti-CD137 moiety was determined by its ability to induce crosslinking dependent CD137 signaling using assay cells overexpressing CD137 and carrying NFκB luciferase reporter.

In some embodiments, the PD-L1/CD137 bispecific exhibits one or more of the following characteristics, alone or in combination:
(a) is specific for human PD-L1 and binds to human CD137;
(b) cross-reacts with cynomolgus PD-L1 and CD137;
(c) disrupts interaction of PD-1 and PD-L1;
(d) disrupts (e.g., reduces or prevents) human CD137L binding to CD137;
(e) exhibits fast on and fast off properties to CD137;
(f) dis-inhibits T cell PD-L1 mediated check-point inhibitory signal;
(g) possesses PD-L1 dependent agonistic activity to CD137 signaling;
(h) activates T cells in PD-L1 dependent manner;
(i) kills PD-L1 expressing tumor cells by activating CD8 T cells;
(j) demonstrates anti-tumor efficacy in a human PD-L1 and CD137 knock-in using MC38-hPD-L1 syngeneic tumor model;
(k) increases CD8+T cells in the tumor microenvironment;
(l) decreases percentage of Treg cells in the tumor microenvironment; and
(m) reduces on-target toxicity outside of tumor microenvironment.

In certain embodiments, the bispecifics have the ability to enhance immune cell proliferation, survival, cytolytic activity CD8 T cells and the secretion of cytokines. In a particular embodiment, the disclosed PD-L1/CD137 bispecifics are shown to activate human T cells in a CMV recall assay, with greater potency than a monospecific antibody combination.

In some embodiments, the binding proteins described herein possess a characteristic selected from the group consisting of: disrupts interaction of PD-1 and PD-L1, removes T cell PD-L1 mediated check-point inhibitory signal, possess PD-L1 dependent agonistic activity to CD137 signaling, activates T cells in PD-L1 dependent manner, kills PD-L1 expressing tumor cells by activating CD8 T cells, demonstrates anti-tumor efficacy in a human PD-L1 and CD137 knock-in using MC38-hPD-L1 syngeneic tumor model, increases CD8+/T cells in the tumor microenvironment, and decreases percentage of Treg cells in the tumor.

In certain embodiments, the bispecifics have the ability to bind to CD137 and PD-L1 expressing cells. In another embodiments, binding of bispecifics to PD-L1 disrupts its interaction with PD-1 which results in restoration of TCR signaling in Jurkat T cells. In addition, PD-L1 bound bispecifics activate CD137 signaling whereas monospecific anti-PD-L1 antibodies alone fail to induce CD137 signaling. Further, they enhance anti-CD3 stimulated IFNγ release in the PBMCs. More particularly, the bispecifics redirect CD8+ to kill PD-L1 expressing tumor cells in vitro. In one embodiment, using a human PD-L1 expressing MC38 mouse tumor in the human CD137 and human PD-L1 double knock-in mice, the disclosed bispecific has demonstrated strong anti-tumor efficacy.

In some embodiments, the binding protein comprises a variable heavy chain sequence that comprises an amino acid sequence with at least about 95%, about 96%, about 97%, about 98%, or about 99%, sequence identity to the amino acid sequence set forth in SEQ ID NOs: 38, 44, 45, 47, or 50. In other embodiments, the binding protein retains the binding and/or functional activity of a binding protein that comprises the variable heavy chain sequence of SEQ ID Nos: 38, 44, 45, 47, or 50. In still further embodiments, the binding protein comprises the variable heavy chain sequence of SEQ ID Nos: 38, 44, 45, 47, or 50, and have one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NOs: 38, 44, 45, 47, or 50 (based on the numbering system of Kabat).

In particular embodiments, the binding protein comprises a variable heavy chain sequence with at least about 95%, about 96%, about 97%, about 98%, or about 99% sequence identity to the binding protein heavy chain variable region sequence set forth in SEQ ID NOs: 38, 44, 45, 47, or 50 comprises one or more conservative amino acid substitutions in a framework region (based on the numbering system of Kabat), and retains the binding and/or functional activity of a binding protein that comprises a variable heavy chain sequence as set forth in SEQ ID NOs: 38, 44, 45, 47, or 50, and a variable light chain sequence as set forth in SEQ ID NOs: 40 or 46.

In some embodiments, the binding protein comprises a variable light chain sequence that comprises an amino acid sequence with at least about 95%, about 96%, about 97%, about 98%, or about 99%, sequence identity to the amino acid sequence set forth in SEQ ID NOs: 40 or 46. In other embodiments, the binding protein retains the binding and/or functional activity of a binding protein that comprises the variable light chain sequence of SEQ ID NOs: 40 or 46. In still further embodiments, the binding protein comprises the variable light chain sequence of SEQ ID Nos: 40 or 46 and have one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NOs: 40 or 46 (based on the numbering system of Kabat).

In particular embodiments, the binding protein comprises a variable light chain sequence with at least about 95%, about 96%, about 97%, about 98%, or about 99% sequence identity to the binding protein light chain variable region sequence set forth in SEQ ID NOs: 40 or 46, comprises one or more conservative amino acid substitutions in a framework region (based on the numbering system of Kabat), and retains the binding and/or functional activity of a binding protein that comprises a variable heavy chain sequence as set forth in SEQ ID NOs: 38, 44, 45, 47, or 50, and a variable light chain sequence as set forth in SEQ ID NOs: 40 or 46.

### PD-L1/TGFβ Bispecifics

In alternative embodiments, the disclosure provides PD-L1/TGFβ bispecifics comprising a PD-L1 antibody scaffold module derived from an anti-PD-L1 antibody and a tumor microenvironment modulator derived from the ECD of the human TGFβRII, wherein the bispecific dis-inhibits the PD-1/PD-L1 checkpoint and neutralizes the immunosuppressive effect of TGFβ in the tumor microenvironment.

In other embodiments, the disclosure provides PD-L1/TGFβ bispecific comprising a PD-L1 antibody scaffold module derived from an anti-PD-L1 antibody and a TGFβ first binding module derived from the ECD of human TGFβ receptor type II, wherein the bispecific binds both PD-L1 and TGFβ and is able to neutralize the biological activities of TGFβ in the TME.

In some embodiments, the PD-L1/TGFβ bispecific 1923Ab20 (FIG. 13L) contains two Fabs from 1923Ab3 that bind PD-L1, human IgG1 Fc with L234A L235A mutations, and two polypeptides encoding the extracellular domain of TGFβRII attaching to the C-terminus of the 1923Ab3 heavy chains. FIG. 14(A) provides a description of the heavy chain and light chains of 1923Ab20. FIG. 15 provides a more detailed description of the subcomponents. TABLE 6 provides the amino acid sequences of the heavy and light chains, SEQ ID NO: 51 and SEQ ID NO: 40, respectively.

TABLE 6
PD-L1/TGFβ Bispecifics

| Bispecific Ref # | Heavy chain (HC) | Light chain (LC) |
|---|---|---|
| 1923Ab20 | SEQ ID NO: 51 | SEQ ID NO: 40 |

The bioactivity of the anti-PD-L1 moiety was determined by its ability to disrupt interaction between PD-1 and PD-L1 to restore TCR signaling using PD-1/PD-L1 blockage assay. The ability of the extracellular domain (ECD) of TGFβRII to neutralize the bioactivity of human TGFβ was determined by its ability to block TGFβ induced signaling cascade using the SBE (SMAD binding element) reporter cells.

In some embodiments, the bispecifics exhibit one or more of the following characteristics, alone or in combination: (a) is specific for human PD-L1 and binds to human TGFβ; (b) disrupts the interaction of PD-1 and PD-L1; (c) dis-inhibits T cell PD-L1 mediated check-point inhibitory signal; (d) binds human TGFβ and neutralizes its biological activities; (e) reduced toxicity outside of tumor microenvironment; (f) increases CD8+T cells in the tumor microenvironment; or (g) decreases the percentage of Treg cells in the tumor microenvironment.

In certain embodiments, the disclosed PD-L1/TGFβ bispecifics have the ability to enhance immune cell proliferation, survival, the cytolytic activity of CD8 T cells and the secretion of cytokines. In a particular embodiment, the disclosed PD-L1/TGFβ bispecifics are shown to activate human T cells in a CMV recall assay, with greater potency than a monospecific antibody combination.

In some embodiments, the binding proteins described herein possess a characteristic selected from the group consisting of: disrupts the interaction of PD-1 and PD-L1, removes T cell PD-L1 mediated check-point inhibitory signal, activates T cells in PD-L1 dependent manner, kills PD-L1 expressing tumor cells by activating CD8 T cells, increases CD8+ T cells in the tumor microenvironment, and decreases the percentage of Treg cells in the tumor.

In some embodiments, the PD-L1/TGFβ bispecifics exhibit one or more of the following functional characteristics, alone or in combination: disrupts the interaction of PD-1 and PD-L1, removes T cell PD-L1 mediated check-point inhibitory signal, inhibits TGFβ signaling.

In a particular embodiment, the disclosed bispecific combined with Urelumab-NR is shown to activate human T cells in a CMV recall assay.

In one embodiment, the disclosed antibodies containing the extracellular domain (ECD) of TGFβRII block TGFβ induced signaling in the HEK cells carrying SBE reporter gene.

In certain embodiments, the bispecifics have the ability to bind to PD-L1 expressing cells. In another embodiments, binding of bispecifics to PD-L1 disrupts its interaction with PD-1 which results in restoration of TCR signaling in Jurkat T cells.

In some embodiments, the binding protein comprises a variable heavy chain sequence that comprises an amino acid sequence with at least about 95%, about 96%, about 97%, about 98%, or about 99%, sequence identity to the amino acid sequence set forth in SEQ ID NO: 51. In other embodiments, the binding protein retains the binding and/or functional activity of a binding protein that comprises the variable heavy chain sequence of SEQ ID NO: 51. In still further embodiments, the binding protein comprises the variable heavy chain sequence of SEQ ID NO: 51 and have one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NO: 51 (based on the numbering system of Kabat).

In particular embodiments, the binding protein comprises a variable heavy chain sequence with at least about 95%, about 96%, about 97%, about 98%, or about 99% sequence identity to the binding protein heavy chain variable region sequence set forth in SEQ ID NO: 51, comprises one or more conservative amino acid substitutions in a framework region (based on the numbering system of Kabat), and retains the binding and/or functional activity of a binding protein that comprises a variable heavy chain sequence as set forth in SEQ ID NO: 51 and a variable light chain sequence as set forth in SEQ ID NO: 40.

In some embodiments, the binding protein comprises a variable light chain sequence that comprises an amino acid sequence with at least about 95%, about 96%, about 97%, about 98%, or about 99%, sequence identity to the amino acid sequence set forth in SEQ ID NO: 40. In other embodiments, the binding protein retains the binding and/or functional activity of a binding protein that comprises the variable light chain sequence of SEQ ID NO: 40. In still further embodiments, the binding protein comprises the variable light chain sequence of SEQ ID NO: 40 and have one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NO: 40 (based on the numbering system of Kabat).

In particular embodiments, the binding protein comprises a variable light chain sequence with at least about 95%, about 96%, about 97%, about 98%, or about 99% sequence identity to the binding protein light chain variable region sequence set forth in SEQ ID NO: 40, comprises one or more conservative amino acid substitutions in a framework region (based on the numbering system of Kabat), and retains the binding and/or functional activity of a binding protein that comprises a variable heavy chain sequence as set forth in SEQ ID NO: 51 and a variable light chain sequence as set forth in SEQ ID NO: 40.

### PD-L1/TGFβ/CD137 Trispecifics

In certain embodiments, a binding protein of the disclosure is trispecific and constructed in the form of a recombinant protein comprising an antibody scaffold module that binds PD-L1 (derived from an antibody), a first binding module comprising a TGFβ receptor II binding protein, and a second binding module that binds CD137 (derived from an antibody).

In particular embodiments, the disclosure provides trispecifics comprising an antibody scaffold module that binds PD-L1 derived from an anti-PD-L1 antibody, a TGFβ first binding module derived from the ECD of TGFβ receptor type 2, and a CD137 second binding module derived from an anti-CD137 antibody, wherein the trispecific binds PD-L1, CD137 and also depletes TGFβ from the local microenvironment thereby activating T cells in a PD-L1-dependent manner.

TABLE 7
PD-L1/TGFβRII/CD137 Trispecifics

| Trispecific | Heavy | Heavy | Light |
|---|---|---|---|
| Ref # | Chain 1 | Chain 2 | Chain |
| 1923Ab7 | SEQ ID NO: 38 | | SEQ ID NO: 39 |
| 1923Ab9 | SEQ ID NO: 41 | SEQ ID NO: 42 | SEQ ID NO: 39 |
| 1923Ab10 | SEQ ID NO: 43 | SEQ ID NO: 42 | SEQ ID NO: 39 |
| 1923Ab17 | SEQ ID NO: 50 | | SEQ ID NO: 39 |
| 1923Ab19 | SEQ ID NO: 51 | | SEQ ID NO: 52 |

In some embodiments, the PD-L1/TGFβ/CD137 trispecific 1923Ab7 (FIG. 13A) contains two Fabs from 1923Ab3 that bind PD-L1, human IgG1 Fc with L234A L235A mutations, two scFv fragments (VH precedes VL) derived from 1923Ab4 attaching to the C-terminus of the 1923Ab3 heavy chain, and two polypeptides encoding the extracellular domain of TGFβRII attaching to the C-terminus of the 1923Ab3 light chains. FIG. 14(B) provides a description of the heavy chain and light chains of 1923Ab7. FIG. 15 provides a more detailed description of the subcomponents of the trispecific. TABLE 7 provides the amino acid sequences of the heavy and light chains, SEQ ID NO: 38 and SEQ ID NO: 39, respectively.

In some embodiments, the PD-L1/TGFβ/CD137 trispecific 1923Ab9 (FIG. 13C) contains two Fabs from 1923Ab3 that bind PD-L1, heterodimeric human IgG1 Fc with L234A L235A mutations and knobs-in-holes” (KiH) mutations, one scFv fragment (VH precedes VL) derived from 1923Ab4 attaching to the C-terminus of the “knob” heavy chain, and two polypeptides encoding the extracellular domain of TGFβRII attaching to the C-terminus of the 1923Ab3 light chain. FIG. 14(B) provides a description of the heavy chain and light chains of 1923Ab9. FIG. 15 provides a more detailed description of the subcomponents of the trispecific. TABLE 7 provides the amino acid sequences of heavy chain 1 (Knob) (SEQ ID NO: 41), heavy chain 2 (Hole) (SEQ ID NO: 42) and light chain (SEQ ID NO: 39).

In some embodiments, the PD-L1/TGFβ/CD137 trispecific 1923Ab10 (FIG. 13D) contains two Fabs from 1923Ab3 that bind PD-L1, heterodimeric human IgG1 Fc with L234A L235A mutations and knobs-in-holes” (KiH) mutations, one scFv fragment (VL precedes VH) derived from 1923Ab4 attaching to the C-terminus of the “knob” heavy chain, and two polypeptides encoding the extracellular domain of TGFβRII attaching to the C-terminus of the Ab3 light chain. FIG. 14(B) provides a description of the heavy chain and light chains of 1923Ab10. FIG. 15 provides a more detailed description of the subcomponents of the trispecific. TABLE 7 provides the amino acid sequences of heavy chain 1 (Knob) (SEQ ID NO: 43), heavy chain 2 (Hole) (SEQ ID NO: 42) and light chain (SEQ ID NO: 39).

In some embodiments, the PD-L1/TGFβ/CD137 trispecific 1923Ab17 (FIG. 13I) contains two Fabs from 1923Ab3 that bind PD-L1, human IgG1 Fc with L234A L235A mutations, two disulfide bond-stabilized scFv fragments (VH precedes VL) derived from 1923Ab4 attaching to the C-terminus of the Ab3 heavy chains, and two polypeptides encoding the extracellular domain of TGFβRII attaching to the C-terminus of the Ab3 light chains. FIG. 14(B) provides a description of the heavy chain and light chains of 1923Ab17. FIG. 15 provides a more detailed description of the subcomponents of the trispecific. TABLE 7 provides the amino acid sequences of the heavy and light chains, SEQ ID NO: 50 and SEQ ID NO: 39, respectively.

In some embodiments, the PD-L1/TGFβ/CD137 trispecific 1923Ab19 (FIG. 13K) contains two Fabs from 1923Ab3 that bind PD-L1, human IgG1 Fc with L234A L235A mutations, 2 polypeptides encoding the extracellular domain of TGFβRII attaching to the C-terminus of the 1923Ab3 light chains, and two disulfide bond-stabilized scFv fragments (VH precedes VL) derived from 1923Ab4 attaching to the C-terminus of the 1923Ab3 heavy chains. FIG. 14(B) provides a description of the heavy chain and light chains of 1923Ab19. FIG. 15 provides a more detailed description of the subcomponents of the trispecific. TABLE 7 provides the amino acid sequences of the heavy and light chains, SEQ ID NO: 51 and SEQ ID NO: 52, respectively.

In order to promote the heterodimerization of recombinants based on natural IgG-like structural scaffolds, knobs-into-holes (KiHs) technology, which involves engineering CH3 domains to create either a “knob” or a “hole” in each heavy chain to promote heterodimerization, has been widely applied. In some embodiments, particularly binding proteins that bind PD-L1 are characterized by an asymmetric design. One example of the “knob” mutation includes T366W in the CH3 domain, and one example of the “hole” mutation includes T366S, L368A, Y407V in the CH3 domain. In an embodiment, a stabilizing disulfide bond may be introduced by an additional S354C mutation in the “knob”, and an additional Y349C mutation in the “hole”. All residue numbers are in EU numbering.

The bioactivity of the PD-L1/TGFβ/CD137 trispecific was determined by corresponding signaling events modulated by PD-L1, TGFβ or CD137. In some aspects the anti PD-L1 moiety was determined by its ability to disrupt interaction between PD-1 and PD-L1 using PD-1/PD-L1 blockage assay. The bioactivity of the anti-CD137 moiety was determined by its ability to induce crosslinking dependent CD137 signaling using assay cells overexpressing CD137 and carrying NFκB luciferase reporter. The bioactivity of the anti-TGF-β moiety was determined by its ability to block TGFβ induced signaling cascade using the SBE (SMAD binding element) reporter cells.

In some embodiments, the PD-L1/TGFβ/CD137 trispecifics exhibit one or more of the following functional characteristics, alone or in combination:
(a) capable of binding to human PD-L1, CD137 and TGFβ;
(b) cross-reacts with cynomolgus PD-L1 and CD137;
(c) disrupts (e.g., reduces or prevents) interaction of PD-1 and PD-L1;
(d) disrupts (e.g., reduces or prevents) human CD137L binding to CD137;
(e) exhibits fast on and fast off properties to CD137;
(f) dis-inhibits T cell PD-L1 mediated check-point inhibitory signal;
(g) inhibits TGFβ signaling and neutralizes it's biological activities;
(h) possess PD-L1 dependent agonistic activity to CD137 signaling;
(pi) activates T cells in PD-L1 dependent manner; and
(j) kills PD-L1 expressing tumor cells by activating CD8 T cells.

In some embodiments, the PD-L1/TGFβ/CD137 trispecifics exhibit one or more of the following functional characteristics, alone or in combination: disrupts interaction of PD-1 and PD-L1, removes T cell PD-L1 mediated check-point inhibitory signal, inhibits TGFβ signaling, possess PD-L1 dependent agonistic activity to CD137 signaling, activates T cells in PD-L1 dependent manner, and kills PD-L1 expressing tumor cells by activating CD8 T cells.

In certain embodiments, the trispecifics have the ability to enhance immune cell proliferation, survival, cytolytic activity CD8 T cells and the secretion of cytokines. In a particular embodiment, the disclosed tripecifics are shown to activate human T cells in a CMV recall assay, with greater potency than a monospecific antibody combination.

In one embodiment, the trispecifics block TGFβ induced signaling in the HEK cells carrying SBE reporter gene.

In certain embodiments, the trispecifics have the ability to bind to CD137 and PD-L1 expressing cells. In another embodiments, binding of trispecifics to PD-L1 disrupts its interaction with PD-1 which results in restoration of TCR signaling in Jurkat T cells. In addition, PD-L1 bound bispecifics activate CD137 signaling. Further, they enhance anti-CD3 stimulated IFNγ release in the PBMCs. More particularly, the trispecifics redirect CD8+ to kill PD-L1 expressing tumor cells in vitro.

In some embodiments, the binding protein comprises a variable heavy chain sequence that comprises an amino acid sequence with at least about 95%, about 96%, about 97%, about 98%, or about 99%, sequence identity to the amino acid sequence set forth in SEQ ID NOs: 38, 41, 42, 43, 50 or 51. In other embodiments, the binding protein retains the binding and/or functional activity of a binding protein that comprises the variable heavy chain sequence of SEQ ID Nos: 38, 41, 42, 43, 50 or 51. In still further embodiments, the binding protein comprises the variable heavy chain sequence of SEQ ID Nos: 38, 41, 42, 43, 50 or 51 and have one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NOs: 38, 41, 42, 43, 50 or 51 (based on the numbering system of Kabat).

In particular embodiments, the binding protein comprises a variable heavy chain sequence with at least about 95%, about 96%, about 97%, about 98%, or about 99% sequence identity to the binding protein heavy chain variable region sequence set forth in SEQ ID NOs: 38, 41, 42, 43, 50 or 51, comprises one or more conservative amino acid substitutions in a framework region (based on the numbering system of Kabat), and retains the binding and/or functional activity of a binding protein that comprises a variable heavy chain sequence as set forth in SEQ ID NOs: 38, 41, 42, 43, 50 or 51 and a variable light chain sequence as set forth in SEQ ID NOs: 39 or 52.

In some embodiments, the binding protein comprises a variable light chain sequence that comprises an amino acid sequence with at least about 95%, about 96%, about 97%, about 98%, or about 99%, sequence identity to the amino acid sequence set forth in SEQ ID NOs: 39 or 52. In other embodiments, the binding protein retains the binding and/or functional activity of a binding protein that comprises the variable light chain sequence of SEQ ID Nos: 39 or 52. In still further embodiments, the binding protein comprises the variable light chain sequence of SEQ ID NOs: 39 or 52 and have one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NOs: 39 or 52 (based on the numbering system of Kabat).

In particular embodiments, the binding protein comprises a variable light chain sequence with at least about 95%, about 96%, about 97%, about 98%, or about 99% sequence identity to the binding protein light chain variable region sequence set forth in SEQ ID NOs: 39 or 52, comprises one or more conservative amino acid substitutions in a framework region (based on the numbering system of Kabat), and retains the binding and/or functional activity of a binding protein that comprises a variable heavy chain sequence as set forth in SEQ ID NOs: 38, 41, 42, 43, 50 or 51 and a variable light chain sequence as set forth in SEQ ID NOs: 39 or 52.

### CD137/TGFβ/PD-L1 Trispecifics

In certain embodiments, a binding protein of the disclosure is trispecific and constructed in the form of a recombinant protein comprising an antibody scaffold module that binds CD137 (derived from an antibody), a first binding module comprising a TGFβ receptor II binding protein, and a second binding module that binds PD-L1 (derived from an antibody).

In particular embodiments, the disclosure provides trispecifics comprising an antibody scaffold module that binds CD137 derived from an anti-CD137 antibody, a TGFβ first binding module derived from the ECD of TGFβ receptor type 2, and a PD-L1 second binding module derived from an anti-PD-L1 antibody, wherein the trispecific binds CD137, PD-L1, and also depletes TGFβ from the local microenvironment thereby activating T cells in a PD-L1-dependent manner.

TABLE 8
CD137/TGFβRII/PD-L1 Trispecifics

| Trispecific Ref # | Heavy Chain | Light Chain |
|---|---|---|
| 1923Ab16 | SEQ ID NO: 48 | SEQ ID NO: 49 |

In some embodiments, the CD137/TGFβRII/PD-L1 trispecific 1923Ab16 (FIG. 13H) contains two Fabs from 1923Ab4 that bind CD137, human IgG1 Fc with L234A L235A mutations, two scFv fragments (VL precedes VH) derived from 1923Ab3 attaching to the C-terminus of the 1923Ab4 heavy chains, and two polypeptides encoding the extracellular domain of TGFβRII attaching to the C-terminus of the Ab4 light chains. FIG. 14(B) provides a description of the heavy chain and light chains of 1923Ab16. FIG. 15 provides a more detailed description of the subcomponents. TABLE 8 provides the amino acid sequences of the heavy and light chains, SEQ ID NO: 48 and SEQ ID NO: 49 respectively.

### Methods of Producing Binding Proteins

The binding proteins disclosed herein may be made by any method known in the art. For example, a recipient may be immunized with soluble recombinant human PD-L1 and/or CD137 protein, or a fragment or a peptide conjugated with a carrier protein thereof. Any suitable method of immunization can be used. Such methods can include adjuvants, other immune stimulants, repeat booster immunizations, and the use of one or more immunization routes.

Any suitable source of human PD-L1 and/or CD137 can be used as the immunogen for the generation of the non-human or human anti-PD-L1 and/or CD137 antibodies of the compositions and methods disclosed herein.

Different forms of the PD-L1 and/or CD137 antigen may be used to generate an antibody that is sufficient to generate a biologically active antibody. Thus, the eliciting PD-L1 and/or CD137 antigen may be a single epitope, multiple epitopes, or the entire protein alone or in combination with one or more immunogenicity enhancing agents. In some aspects, the eliciting antigen is an isolated soluble full-length protein, or a soluble protein comprising less than the full-length sequence (e.g., immunizing with a peptide comprising particular portion or epitopes of PD-L1 and/or CD137). As used herein, the term “portion” refers to the minimal number of amino acids or nucleic acids, as appropriate, to constitute an immunogenic epitope of the antigen of interest. Any genetic vectors suitable for transformation of the cells of interest may be employed, including, but not limited to adenoviral vectors, plasmids, and non-viral vectors, such as cationic lipids.

It is desirable to prepare monoclonal antibodies (mAbs) from various mammalian hosts, such as mice, rodents, primates, humans, etc. Description of techniques for preparing such monoclonal antibodies may be found in, e.g., Sties et al. (eds.) BASIC AND CLINICAL IMMUNOLOGY (4^{th} ed.) Lance Medical Publication, Los Altos, CA, and references cited therein; Harlow and Lane (1988) ANTIBODIES: A LABORATORY MANUAL CSH Press; Goding (1986) MONOCLONAL ANTIBODIES: PRINCIPLES AND PRACTICE (2^{nd} ed.) Academic Press, New York, NY. Typically, spleen cells from an animal immunized with a desired antigen are immortalized, commonly by fusion with a myeloma cell. See Kohler and Milstein (196) Eur. J. Immunol. 6:511-519. Alternative methods of immortalization include transformation with Epstein Barr Virus, oncogene, or retroviruses, or other methods known in the art. See. e.g., Doyle et al. (eds. 1994 and periodic supplements) CELL AND TISSUE CULTURE: LABORATORY PROCEDURES, John Wiley and Sons, New York, NY. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or an antigen binding fragment thereof by screening a DNA library from human B cells according, e.g., to the general protocol outlined by Huse et al., (1989) Science 246: 1275-1281. Thus, antibodies may be obtained by a variety of techniques familiar to researchers skilled in the art.

Other suitable techniques involve selection of libraries of antibodies in phage, yeast, virus or similar vector. See e.g., Huse et al., supra; and Ward et al., (1989) Nature 341:544-546. The polypeptides and antibodies disclosed herein may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literatures. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,996,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86: 10029-10023; or made in transgenic mice, see Nils Lonberg et al., (1994), Nature 368:856-859; and Mendez et al. (1997) Nature Genetics 15: 146-156; TRANSGENIC ANIMALS AND METHODS OF USE (WO 2012/62118), Medarex, Trianni, Abgenix, Ablexis, OminiAb, Harbour and other technologies.

In some embodiments, the ability of the produced antibody to bind to PD-L1 and/or CD137 can be assessed using standard binding assays, such as surface plasmon resonance (SPR), ELISA, Western Blot, immunofluorescence, flow cytometric analysis, chemotaxis assays, and cell migration assays. In some aspects, the produced antibody may also be assessed for its ability to inhibit PD-L1 and/or activate CD137 from blocking PD-L1 and/or activating CD137 receptor signal transduction.

The antibody composition prepared from the cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (see, e.g., Lindmark et al., 1983 J. Immunol. Meth. 62:1-13). Protein G is recommended for all mouse isotypes and for human γ3 (see, e.g., Guss et al., 1986 EMBO J. 5:1567-1575). A matrix to which an affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25 M salt).

Also included are nucleic acids that hybridize under low, moderate, and high stringency conditions, as defined herein, to all or a portion (e.g., the portion encoding the variable region) of the nucleotide sequence represented by isolated polynucleotide sequence(s) that encode an antibody or antibody fragment of the present disclosure. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, or at least 98%, identical to the sequence of a portion or all of a nucleic acid encoding an anti-PD-L1 and/or CD137 polypeptide (e.g., a heavy chain or light chain variable region), or its complement. Hybridizing nucleic acids of the type described herein can be used, for example, as a cloning probe, a primer, e.g., a PCR primer, or a diagnostic probe.

### Polynucleotides, Vectors, and Cells

Other embodiments encompass isolated polynucleotides that comprise a sequence encoding a binding protein or fragment thereof as disclosed herein, vectors and cells comprising the polynucleotides, and recombinant techniques for production of the disclosed binding proteins. The isolated polynucleotides can encode any desired form of the binding protein including, for example, full length monoclonal antibodies, Fab, Fab′, F(ab′)2, and Fv fragments, diabodies, linear antibodies, single-chain antibody molecules, miniantibodies.

Some embodiments include isolated polynucleotides comprising sequences that encode the heavy chain variable region of a binding protein or fragment thereof having the amino acid sequence of any of SEQ ID NOs: 1, 3, 16, 18, and 20. Some embodiments include isolated polynucleotides comprising sequences that encode the light chain variable region of a binding protein or fragment thereof having the amino acid sequence of any of SEQ ID NOs: 2, 4, 17, 19, and 21.

In an embodiment, the isolated polynucleotide sequence(s) encode a binding protein or fragment thereof having a heavy chain variable region and a light chain variable region comprising the amino acid sequences of: (a) a heavy chain variable region sequence comprising SEQ ID NO: 1 and a light chain variable region sequence comprising SEQ ID NO: 2; (b) a heavy chain variable region sequence comprising SEQ ID NO: 3 and a light chain variable region sequence comprising SEQ ID NO: 4; (c) a heavy chain variable region sequence comprising SEQ ID NO: 16 and a light chain variable region sequence comprising SEQ ID NO: 17; (d) a heavy chain variable region sequence comprising SEQ ID NO: 18 and a light chain variable region sequence comprising SEQ ID NO: 19; or (e) a heavy chain variable region sequence comprising SEQ ID NO: 20 and a light chain variable region sequence comprising SEQ ID NO: 21.

In another embodiment, the isolated polynucleotide sequence encodes a binding protein or fragment thereof having a heavy chain variable region and a light chain variable region comprising the amino acid sequences of: (a) a heavy chain variable region sequence that is 90%, 95%, or 99% identical to SEQ ID NO: 1 and a light chain variable region sequence that is 90%, 95%, or 99% identical to SEQ ID NO: 2; (b) a heavy chain variable region sequence that is 90%, 95%, or 99% identical to SEQ ID NO: 3 and a light chain variable region sequence that is 90%, 95%, or 99% identical to SEQ ID NO: 4; (c) a heavy chain variable region sequence that is 90%, 95%, or 99% identical to SEQ ID NO: 16 and a light chain variable region sequence that is 90%, 95%, or 99% identical to SEQ ID NO: 17; (d) a heavy chain variable region sequence that is 90%, 95%, or 99% identical to SEQ ID NO: 18 and a light chain variable region sequence that is 90%, 95%, or 99% identical to SEQ ID NO: 19; or (e) a heavy chain variable region sequence that is 90%, 95%, or 99% identical to SEQ ID NO: 20 and a light chain variable region sequence that is 90%, 95%, or 99% identical to SEQ ID NO: 21.

The polynucleotide(s) that comprise a sequence encoding a binding protein or fragment thereof as disclosed herein can be fused to one or more regulatory or control sequence, as known in the art, and can be contained in suitable expression vectors or cells as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the binding protein or fragment thereof is inserted into a replicable vector for cloning (amplification of the DNA) or for expression. Many suitable vectors for expressing the binding protein or fragment thereof are available. The vector components generally include, but are not limited to, one or more of the following: a signal sequence, an origin of replication, one or more marker genes, an enhancer element, a promoter, and a transcription termination sequence.

The binding protein or fragment thereof disclosed herein can also be produced as fusion polypeptides, in which the binding protein is fused with a heterologous polypeptide, such as a signal sequence or other polypeptide having a specific cleavage site at the amino terminus of the mature protein or polypeptide. The heterologous signal sequence selected is typically one that is recognized and processed (i.e., cleaved by a signal peptidase) by the cell. For prokaryotic cells, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO 90/13646. In mammalian cells, mammalian signal sequences as well as viral secretory leaders, for example, the herpes simplex gD signal, can be used. The DNA for such precursor region is ligated in reading frame to DNA encoding the binding protein or fragment thereof.

Expression and cloning vectors contain a nucleic acid sequence that enables the vector to replicate in one or more selected cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

### Cell Culture

The cells used to produce the binding proteins or fragments thereof as disclosed herein may be cultured in a variety of media. Commercially available media such as Ham's F10 (Sigma), Minimal Essential Medium ((MEM), Sigma), RPMI-1640 (Sigma), FreeStyle™ (Cibco) and Dulbecco's Modified Eagle's Medium ((DMEM), Sigma) are suitable for culturing the host cells. Any of these or other media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin), trace elements (such as inorganic compounds usually present at final concentrations in the micromolar or lower range), and glucose or an equivalent energy source. Any other necessary supplements may also be included at appropriate concentrations that would be known to those skilled in the art. The culture conditions, such as temperature, pH, and the like, include those previously used with the cell selected for expression, and will be apparent to those skilled in the art.

### Non-Therapeutic Uses

The binding proteins described herein are useful as affinity purification agents. In this process, a binding protein is immobilized on a solid phase such a Protein A resin, using methods well known in the art. The immobilized binding protein is contacted with a sample containing PD-L1, TGFβ, and/or CD137 protein (or a fragment thereof) to be purified, and thereafter the support is washed with a suitable solvent that will remove substantially all the material in the sample except the PD-L1, TGFβ, and/or CD137 protein, which is bound to the immobilized binding protein. Finally, the support is washed with another suitable solvent that will release the PD-L1, TGFβ, and/or CD137 protein from the binding protein.

The binding proteins disclosed herein are also useful in diagnostic assays to detect and/or quantify PD-L1, TGFβ, and/or CD137 protein, for example, detecting PD-L1, TGFβ, and/or CD137 expression in specific cells, tissues, or serum. The binding proteins can be used diagnostically to, for example, monitor the development or progression of a disease as part of a clinical testing procedure to, e.g., determine the efficacy of a given treatment and/or prevention regimen. Detection can be facilitated by coupling the binding protein to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to binding proteins for use as diagnostics according to the present disclosure.

The binding proteins can be used in methods for diagnosing a PD-L1, TGFβ, and/or CD137-associated disorder (e.g., a disorder characterized by abnormal expression of PD-L1, TGFβ, and/or CD137) or to determine if a subject has an increased risk of developing a PD-L1, TGFβ, and/or CD137-associated disorder. Such methods include contacting a biological sample from a subject with a binding protein disclosed herein and detecting binding of the molecule to PD-L1, TGFβ, and/or CD137. By “biological sample” is intended any biological sample obtained from an individual, cell line, tissue culture, or other source of cells potentially expressing PD-L1, TGFβ, and/or CD137. Methods for obtaining tissue biopsies and body fluids from mammals are well known in the art.

In some embodiments, the method can further comprise comparing the level of PD-L1, TGFβ, and/or CD137 in a patient sample to a control sample (e.g., a subject that does not have a PD-L1, TGFβ, and/or CD137-associated disorder) to determine if the patient has a PD-L1, TGFβ, and/or CD137-associated disorder or is at risk of developing a PD-L1, TGFβ, and/or CD137-associated disorder.

It will be advantageous in some embodiments, for example, for diagnostic purposes to label a binding protein with a detectable moiety. Numerous detectable labels are available, including radioisotopes, fluorescent labels, enzyme substrate labels and the like. The label may be indirectly conjugated with the binding protein using various known techniques. For example, the binding protein can be conjugated with biotin and any of the three broad categories of labels mentioned above can be conjugated with avidin, or vice versa. Biotin binds selectively to avidin and thus, the label can be conjugated with the binding protein in this indirect manner. Alternatively, to achieve indirect conjugation of the label with the binding protein, the binding protein can be conjugated with a small hapten (such as digoxin) and one of the different types of labels mentioned above is conjugated with an anti-hapten antibody (e.g., anti-digoxin antibody). Thus, indirect conjugation of the label with the binding protein can be achieved.

Exemplary radioisotopes labels include ³⁵S, ¹⁴C, ¹²⁵I, ³H, and ¹³¹I. The binding protein can be labeled with the radioisotope, using the techniques described in, for example, Current Protocols in Immunology, Volumes 1 and 2, 1991, Coligen et al., Ed. Wiley-Interscience, New York, N.Y., Pubs. Radioactivity can be measured, for example, by scintillation counting.

Exemplary fluorescent labels include labels derived from rare earth chelates (europium chelates) or fluorescein and its derivatives, rhodamine and its derivatives, dansyl, Lissamine, phycoerythrin, and Texas Red are available. The fluorescent labels can be conjugated to the binding protein via known techniques, such as those disclosed in Current Protocols in Immunology, for example. Fluorescence can be quantified using a fluorimeter.

There are various well-characterized enzyme-substrate labels known in the art (see, e.g., U.S. Pat. No. 4,275,149). The enzyme generally catalyzes a chemical alteration of the chromogenic substrate that can be measured using various techniques. For example, alteration may be a color change in a substrate that can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured, using a chemiluminometer, for example, or donates energy to a fluorescent acceptor.

Examples of enzymatic labels include luciferases such as firefly luciferase and bacterial luciferase (U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (such as glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to binding protein are described, for example, in O'Sullivan et al., 1981, Methods for the Preparation of Enzyme-Antibody Conjugates for use in Enzyme Immunoassay, in Methods in Enzym. (J. Langone & H. Van Vunakis, eds.), Academic press, N.Y., 73: 147-166.

Examples of enzyme-substrate combinations include, for example: Horseradish peroxidase (HRPO) with hydrogen peroxidase as a substrate, wherein the hydrogen peroxidase oxidizes a dye precursor such as orthophenylene diamine (OPD) or 3,3,5,5-tetramethyl benzidine hydrochloride (TMB); alkaline phosphatase (AP) with para-Nitrophenyl phosphate as chromogenic substrate; and β-D-galactosidase (β-D-Gal) with a chromogenic substrate such as p-nitrophenyl-β-D-galactosidase or fluorogenic substrate 4-methylumbelliferyl-β-D-galactosidase.

In another embodiment, a binding protein disclosed herein is used unlabeled and detected with a labeled antibody that binds the binding protein.

The binding proteins described herein may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987).

The binding protein disclosed herein can be used to inhibit the binding of PD-L1, TGFβ, and/or CD137 to its respective receptor. Such methods comprise administering a binding protein disclosed herein to a cell (e.g., a mammalian cell) or cellular environment, whereby signaling mediated by the receptor is inhibited. These methods can be performed in vitro or in vivo. By “cellular environment” is intended the tissue, medium, or extracellular matrix surrounding a cell.

### Compositions and Methods of Treatment

The disclosure also provides compositions including, for example, pharmaceutical compositions that comprise a binding protein disclosed herein. Such compositions have numerous therapeutic uses for the treatment, prevention, or amelioration of diseases or disorders such as cancer.

The present disclosure also provides methods for the treatment or prevention of cancer comprising administering a composition or formulation that comprises a binding protein disclosed herein, and optionally another immune-based therapy, to a subject in need thereof.

The disclosed binding proteins are also useful in methods of treatment of cancer, either alone (e.g., as monotherapies) or in combination with other immunotherapeutic agents and/or a chemotherapy.

The binding proteins can be administered either alone or in combination with other compositions that are useful for treating an immune-mediated inflammatory disorder or an autoimmune disease.

In some aspects, a composition, e.g., a pharmaceutical composition is provided that comprises one or more binding proteins disclosed herein. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed in unit dosage form, for example, as a dry lyophilized powder or water free concentrate in a hermetically sealed container such as an ampoule or sachette indicating the quantity of the active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

A composition can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978.

Dosage levels of the active ingredients in the pharmaceutical compositions may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical compositions described herein may be administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease.

In some aspects, the compositions described herein are administered to patients, e.g., in vivo, to treat or prevent a variety of disorders such as those described herein. Preferred patients include human patients having disorders that can be corrected or ameliorated by administering the binding proteins disclosed herein.

In some aspects, conventional viral and non-viral based gene transfer methods can be used to introduce nucleic acids encoding the antibodies or derivatives thereof, as described herein, in mammalian cells or target tissues. Such methods can be used to administer nucleic acids encoding the antibodies to cells in vitro. In some embodiments, the nucleic acids encoding the antibodies or derivatives thereof are administered for in vivo or ex vivo gene therapy uses. In other embodiments, gene delivery techniques are used to study the activity of the antibodies in cell based or animal models. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Such methods are well known in the art.

Methods of non-viral delivery of nucleic acids encoding engineered polypeptides of the disclosure include lipofection, microinjection, biolistics, virosomes, liposomes, immunoliposomes, polycation or lipid:nucleic acid conjugates, naked DNA, artificial virions, and agent-enhanced uptake of DNA. Lipofection methods and lipofection reagents are well known in the art (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid:nucleic acid complexes, including targeted liposomes such as immunolipid complexes, is well known to one of skill in the art.

The use of RNA or DNA viral based systems for the delivery of nucleic acids encoding the antibodies described herein take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the disclosure could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues. All patents and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### General Methods

Methods for protein purification including immunoprecipitation, chromatography, and electrophoresis are described. See, e.g., Coligan et al. (2000) Current Protocols in Protein Science, Vol. 1, John Wiley and Sons, Inc., New York. Chemical analysis, chemical modification, post-translational modification, production of fusion proteins, and glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra.

Hybridoma or cell culture supernatant containing an anti-PD-L1 or anti-CD137 antibody was purified via HiTrap protein G column (GE, cat. No. 17040401) according to the manufacturer's procedures. Briefly, supernatant was equilibrated with DPBS (Gibco, cat. No. 14190-136) for 5 CV and loaded via syringe/infusion pump (Legato 200, KDS) at ambient temperature and 3 minute residence time. The column was washed with 5 CV of DPBS and elution was performed with 4 CV of pH 2.8 elution buffer (Fisher Scientific, cat. No. PI21004). Elution was fractionated, and fractions were neutralized with 1M Tris-HCL, pH 8.5 (Fisher Scientific, cat No. 50-843-270) and assayed by A280 (DropSense96, Trinean). Peak fractions were pooled, and buffer exchanged into DPBS. Centrifugal filters (EMD Millipore, cat. No. UFC803024) were equilibrated in DPBS at 4,000×g for 2 mins. Purified sample was loaded, DPBS was added and the sample was spun at 4,000×g for 5-10 minute spins until total DPBS volume reached ≥6 DV. The final pool was analyzed by A280.

Standard methods in molecular biology are described. See, e.g., Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Stable cell lines expressing human PD-L1 and CD137 were generated by transfecting a selected host cell (i.e., CHO-K1 or HEK293) with pcDNA3.1-based plasmids expressing Homo sapiens target proteins using electroporation- or lipid-based transfection. Geneticin or Puromycin was used to select the integrated cells. After 7-10 days of antibiotic selection, stable clones were isolated by FACS or serial dilution using a labelled antibody. After expansion, the stable clones were further confirmed for target protein expression by flow cytometry. Mouse and cynomolgus targets were respectively transiently expressed in HEK293T cells using lipid-based transfection.

The sequences for the heavy and light chain variable regions for hybridoma clones were determined as described below. Total RNA was extracted from 1-2×10⁶ hybridoma cells using the RNeasy Plus Mini Kit from Qiagen (Germantown, MD, USA). CDNA was generated by performing 5′ RACE reactions using the SMARTer RACE 5′/3′ Kit from Takara (Mountainview, CA, USA). PCR was performed using the Q5 High-Fidelity DNA Polymerase from NEB (Ipswich, MA, USA) to amplify the variable regions from the heavy and light chains using the Takara Universal Primer Mix in combination with gene specific primers for the 3′ mouse constant region of the appropriate immunoglobulin. The amplified variable regions for the heavy and light chains were run on 2% agarose gels, the appropriate bands excised and then gel purified using the Mini Elute Gel Extraction Kit from Qiagen. The purified PCR products were cloned using the Zero Blunt PCR Cloning Kit from Invitrogen (Carlsbad, CA, USA), transformed into Stellar Competent E. coli cells from Takara and plated onto LB Agar+50 ug/ml kanamycin plates. Direct colony Sanger sequencing was performed by GeneWiz (South Plainfield, NJ, USA). The resulting polynucleotide sequences were analyzed using IMGT V-QUEST to identify productive rearrangements and analyze translated protein sequences. CDR determination was based on Kabat numbering.

Selected VH or VL chains were PCR amplified and cloned into a pcDNA3.4-based expression vector, which harbors the constant region from human IgG1 (Uniprot P01857) or human Kappa light chain (UniProt P01834). Paired heavy chain- and light chain-expressing plasmids were transfected into Expi293 cells (Thermo Fisher Scientific) following provider's Expi293 expression system protocol. Five days after transfection culture supernatants were collected by centrifugation. Expressed antibodies were purified by 1-step affinity purification using Protein A column and buffer exchanged to PBS pH 7.2.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard techniques for characterizing ligand/receptor interactions are available. See, e.g., Coligan et al. (2001) Current Protocols in Immunology, Vol. 4, John Wiley, Inc., New York. Standard methods of antibody functional characterization appropriate for the characterization of antibodies with particular mechanisms of action are also well known to those of skill in the art.

Software packages and databases for determining, e.g., antigenic fragments, leader sequences, protein folding, functional domains, CDR annotation, glycosylation sites, and sequence alignments, are available.

An in-house anti-PD-L1 antibody based on the anti-PD-L1 antibody (Avelumab) referred to herein as “Avelumab-NR” (PC1), was prepared based on the publicly available information published in U.S. Pat. No. 10,759,856 (VH SEQ ID NO: 24 and VL SEQ ID NO: 25 therein). The PC1 antibody was used to establish the functional assays used to evaluate and characterize the anti-PD-L1 specific antibodies disclosed herein.

An in-house anti-CD137 antibody based on the anti-CD137 antibody (Urelumab) referred to herein as “Urelumab-NR” (PC2), was prepared based on the publicly available information published in U.S. Pat. No. 7,288,638 (VH SEQ ID NO: 3 and VL SEQ ID NO: 6 therein). A second in-house CD137 reactive antibody (Utomilumab), referred to herein as “Utomilumab-NR” (PC3), was prepared based on publicly available information published in U.S. Pat. No. 8,337,850 (VH SEQ ID NO: 43 and VL SEQ ID NO: 45 therein). The PC2 and PC3 antibodies were used to confirm CD137 expression in cell lines used in the examples and to establish the binding and functional assays used to evaluate and characterize the anti-CD137 specific antibodies disclosed herein. Example 18 and 22 in this application illustrate that the disclosed bispecifics and trispecifics induced stronger CD137 signaling and T cell activation compared to Urelumab-NR and Utomilumab-NR.

Reference sequences utilized herein are illustrated in Table 9

TABLE 9
Reference Sequences

| SEQ ID NO: | NCBI Ref. Seq | Amino Acid Sequence |
|---|---|---|
| SEQ ID NO: | NP_054862.1 | MRIFAVFIFMTYWHLLNAFTVTVPKDLYVVEY |
| 68 | Human PD-L1 | GSNMTIECKFPVEKQLDLAALIVYWEMEDKNI |
| | | IQFVHGEEDLKVQHSSYRQRARLLKDQLSLGN |
| | | AALQITDVKLQDAGVYRCMISYGGADYKRIT |
| | | VKVNAPYNKINQRILVVDPVTSEHELTCQAEG |
| | | YPKAEVIWTSSDHQVLSGKTTTTNSKREEKLF |
| | | NVTSTLRINTTTNEIFYCTFRRLDPEENHTAEL |
| | | VIPELPLAHPPNERTHLVILGAILLCLGVALTFIF |
| | | RLRKGRMMDVKKCGIQDTNSKKQSDTHLEET |
| SEQ ID NO: | XP_005581836.1 | MRIFAVFIFTYWHLLNAFTVTVPKDLYVVEY |
| 69 | Cyno PD-L1 | GSNMTIECKFPVEKQLDLTSLIVYWEMEDKNII |
| | [predicted] | QFVHGEEDLKVQHSNYRQRAQLLKDQLSLGN |
| | | AALRITDVKLQDAGVYRCMISYGGADYKRITV |
| | | KVNAPYNKINQRILVVDPVTSEHELTCQAEGY |
| | | PKAEVIWTSSDHQVLSGKTTTTNSKREEKLLN |
| | | VTSTLRINTTANEIFYCIFRRLDPEENHTAELVI |
| | | PELPLALPPNERTHLVILGAIFLLLGVALTFIFY |
| | | LRKGRMMDMKKCGIRVTNSKKQRDTQLEET |
| SEQ ID NO: | NP_068693.1 | MRIFAGIIFTACCHLLRAFTITAPKDLYVVEYG |
| 70 | Murine PD-L1 | SNVTMECRFPVERELDLLALVVYWEKEDEQVI |
| | | QFVAGEEDLKPQHSNFRGRASLPKDQLLKGN |
| | | AALQITDVKLQDAGVYCCIISYGGADYKRITL |
| | | KVNAPYRKINQRISVDPATSEHELICQAEGYPE |
| | | AEVIWTNSDHQPVSGKRSVTTSRTEGMLLNVT |
| | | SSLRVNATANDVFYCTFWRSQPGQNHTAELIIP |
| | | ELPATHPPQNRTHWVLLGSILLFLIVVSTVLLF |
| | | LRKQVRMLDVEKCGVEDTSSKNRNDTQFEET |
| SEQ ID NO: | NP_001552.2 | MGNSCYNIVATLLLVLNFERTRSLQDPCSNCP |
| 71 | Human CD137 | AGTFCDNNRNQICSPCPPNSFSSAGGQRTCDIC |
| | | RQCKGVFRTRKECSSTSNAECDCTPGFHCLGA |
| | | GCSMCEQDCKQGQELTKKGCKDCCFGTFNDQ |
| | | KRGICRPWTNCSLDGKSVLVNGTKERDVVCG |
| | | PSPADLSPGASSVTPPAPAREPGHSPQIISFFLAL |
| | | TSTALLFLLFFLTLRFSVVKRGRKKLLYIFKQPF |
| | | MRPVQTTQEEDGCSCRFPEEEEGGCEL |
| SEQ ID NO: | XP_005544945.1 | MGNSCYNIVATLLLVLNFERTRSLQDLCSNCP |
| 72 | Cyno CD137 | AGTFCDNNRSQICSPCPPNSFSSAGGQRTCDIC |
| | [predicted] | RQCKGVFKTRKECSSTSNAECDCISGYHCLGA |
| | | ECSMCEQDCKQGQELTKKGCKDCCFGTENDQ |
| | | KRGICRPWTNCSLDGKSVLVNGTKERDVVCG |
| | | PSPADLSPGASSATPPAPAREPGHSPQIIFFLAL |
| | | TSTVVLFLLFFLVLRFSVVKRSRKKLLYIFKQP |
| | | FMRPVQTTQEEDGCSCRFPEEEEGGCEL |
| SEQ ID NO: | NP_001070977.1 | MGNSCYNIVATLLLVLNFERTRSLQDPCSNCP |
| 73 | Murine CD137 | AGTFCDNNRNQICSPCPPNSFSSAGGQRTCDIC |
| | | RQCKGVFRTRKECSSTSNAECDCTPGFHCLGA |
| | | GCSMCEQDCKQGQELTKKGCKDCCFGTENDQ |
| | | KRGICRPWTNCSLDGKSVLVNGTKERDVVCG |
| | | PSPADLSPGASSVTPPAPAREPGHSPQIISFFLAL |
| | | TSTALLFLLFFLTLRFSVVKRGRKKLLYIFKQPF |
| | | MRPVQTTQEEDGCSCRFPEEEEGGCEL |
| SEQ ID NO: | NP_003233.4 | MGRGLLRGLWPLHIVLWTRIASTIPPHVQKSV |
| 74 | Human TGF- | NNDMIVTDNNGAVKFPQLCKFCDVRFSTCDN |
| | beta receptor | QKSCMSNCSITSICEKPQEVCVAVWRKNDENI |
| | type-2 isoform | TLETVCHDPKLPYHDFILEDAASPKCIMKEKK |
| | B | KPGETFFMCSCSSDECNDNIIFSEEYNTSNPDLL |
| | | LVIFQVTGISLLPPLGVAISVIIIFYCYRVNRQQK |
| | | LSSTWETGKTRKLMEFSEHCAIILEDDRSDISST |
| | | CANNINHNTELLPIELDTLVGKGRFAEVYKAK |
| | | LKQNTSEQFETVAVKIFPYEEYASWKTEKDIFS |
| | | DINLKHENILQFLTAEERKTELGKQYWLITAFH |
| | | AKGNLQEYLTRHVISWEDLRKLGSSLARGIAH |
| | | LHSDHTPCGRPKMPIVHRDLKSSNILVKNDLT |
| | | CCLCDFGLSLRLDPTLSVDDLANSGQVGTARY |
| | | MAPEVLESRMNLENVESFKQTDVYSMALVLW |
| | | EMTSRCNAVGEVKDYEPPFGSKVREHPCVES |
| | | MKDNVLRDRGRPEIPSFWLNHQGIQMVCETLT |
| | | ECWDHDPEARLTAQCVAERFSELEHLDRLSGR |
| | | SCSEEKIPEDGSLNTTK |

### Example 1: Generation of Binding Proteins that Bind PD-L1

Fully human anti-human PD-L1 antibodies were generated by immunizing human Ig transgenic mice, Trianni mice that express human antibody VH and VL genes (see, e.g., WO 2013/063391, TRIANNI® mice).

Immunization-TRIANNI mice described above were immunized by injection with recombinant human PD-L1 protein via intraperitoneally (IP), subcutaneously (SC), based of tail or footpad injections.

The immune response was monitored by retroorbital bleeds. The plasma was screened by ELISA, flow cytometry (FACS) or Imaging (as described below). Mice with sufficient anti-PD-L1 titers were used for fusions. Mice were boosted intraperitoneally, at the base of the tail, footpad or intravenously with the immunogen before sacrifice and removal of the spleen and lymph nodes.

Selection of Mice Producing Anti-PD-L1 Antibodies—to select mice producing antibodies that bound PD-L1, sera from immunized mice were screened by ELISA, FACS or imaging for binding to human PD-L1 protein, cells expressing PD-L1 protein (HEK293T transfected with the PD-L1 gene) not the control cells that do not express PD-L1 (HEK293T cells).

For ELISA, briefly, an ELISA plate coated with recombinant human PD-L1 (AcroBiosystems, catalog number: PD1-H5229) was incubated with dilutions of serum from immunized mice for one hour at room temperature, the assay plate was washed, and specific antibody binding was detected with HRP-labeled anti-mouse IgG antibody (Jackson ImmunoResearch, catalog number: 109-035-088) after one hour incubation at room temperature, washed, and followed by ABTS substrate (Moss, catalog number: ABTS-1000) incubation for 30 minutes at room temperature. Plate was read using an ELISA plate reader (Biotek).

For FACS, briefly, PD-L1-HEK293T cells or parental HEK293T cells were incubated with dilutions of serum from immunized mice for 2 hours at 4° C. Cells were fixed with 2% PFA (Alfa Aesar, catalog number: J61899) for 15 minutes at 4° C. and then washed. Specific antibody binding was detected with Alexa 647 labeled goat anti-mouse IgG antibody (ThermoFisher Scientific, catalog number: A-21445) after one-hour incubation at 4° C. Flow cytometric analyses were performed on a flow cytometry instrument (Intellicyte, IQue plus, Sartorius).

In addition, mice serum was tested by imaging. Briefly, PD-L1-HEK293T cells were incubated with dilutions of serum from immunized mice. Cells were washed, fixed with paraformaldehyde, washed, specific antibody binding was detected with secondary Alexa488 goat anti-mouse antibody and Hoechst (Invitrogen). Plates were scanned and analyzed on an imaging machine (Cytation 5, Biotek).

Generation of Hybridomas Producing Antibodies to PD-L1—to generate hybridomas producing human antibodies of the invention, splenocytes and lymph node cells were isolated from an immunized mouse and fused to an appropriate immortalized cell line, such as a mouse myeloma cell line. The resulting hybridomas were screened for the production of antigen-specific antibodies. For example, single cell suspensions of splenocytes, lymph node cells from immunized mice were fused to equal number of Sp2/0 non-secreting mouse IgG myeloma cells (ATCC, CRL 1581) by electrofusion. Cells were plated in flat bottom 96-well tissue culture plates, followed by about one week of incubation in selection medium (HAT medium), then switched to hybridoma culture media. Approximately 10-14 days after cell plating, supernatants from individual wells were screened by ELISA, Imaging or FACS as described above. The antibody secreting-hybridomas were transferred to 24-well plates, screened again, and if still positive for anti-PD-L1, the positive hybridomas were subcloned by sorting using a single cell sorter. The subclones were screened again by ELISA, Imaging or FACS as described above. The stable subclones were then cultured in vitro to generate small amounts of antibodies for purification and characterization.

### Example 2: Binding Specificity of mAbs that Bind PD-L1 to Human, Mouse and Cynomolgus PD-L1 Proteins

The binding specificity of disclosed anti-PD-L1 antibodies (1923Ab2 and 1923Ab3) to different species of PD-L1 protein was assessed by ELISA. Briefly, human recombinant PD-L1 protein (Acro Biosystems, cat #: PD1-H5229), cynomolgus PD-L1 protein (Acro Biosystems, cat #: PD1-C52H4) and mouse PD-L1 protein (Acro Biosystems, cat #: PD1-M5220) were directly coated to ELISA plates. 1923Ab2 and 1923Ab3 was then added to the plates followed by detection by Peroxidase AffiniPure F(ab′)₂ Fragment Goat Anti-Mouse IgG, Fcγ fragment specific (Jackson ImmunoResearch, cat #: 115-036-071) or Peroxidase AffiniPure F(ab′)₂ Fragment Goat Anti-human IgG, Fcγ fragment specific (Jackson ImmunoResearch, cat #: 109-036-098). After addition of ABTS substrate (Moss, cat #: ABTS-1000), ELISA plates were read using a Synergy Neo2 Multi-mode reader (Bioteck, SN #: 180213F).

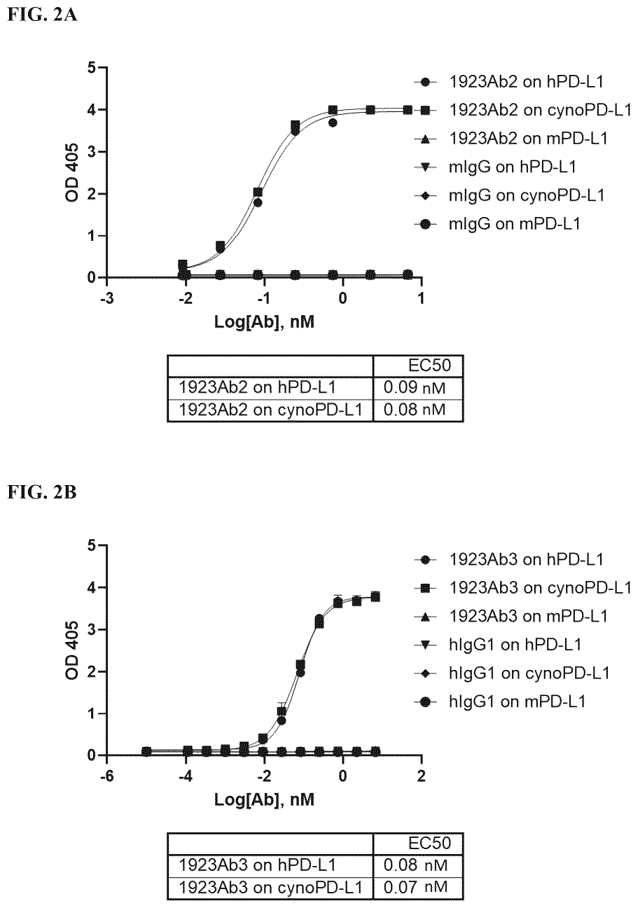

FIGS. 2A&B shows the binding activities of the disclosed anti-PD-L1 antibodies to human and cynomolgus PD-L1 proteins in a dose-dependent manner but not to mouse PD-L1; the isotype control mIgG or hIgG1 does not bind to any species PD-L1. The ELISA binding EC50 values of the disclosed anti-PD-L1 antibodies to human, cynomolgus PD-L1 proteins are provided in FIG. 2A and FIG. 2B.

### Example 3: Binding Affinity of Recombinant Anti-PD-L1 to Human PD-L1

Recombinant anti-PD-L1 antibodies were expressed and purified from Expi293 with the constant region of a human IgG1 or a human IgG1 variant. The binding affinity of the anti-PD-L1 antibodies were evaluated using an immunofluorescence imaging assay.

The cellular binding affinity of anti-PD-L1 antibodies was tested on HEK293T-PD-L1 cells. The cells were plated in complete media containing DMEM with 10% FBS, then incubated overnight at 37° C. Anti-PD-L1 antibodies were serial diluted and added to the assay plates, incubated at 4° C. for 2 hours, followed by fixing cells for 15 minutes at room temperature. The fixed cells were washed with PBS for three times following by staining at room temperature for 1 hour with Alexa Fluor® 488 Goat Anti-Human IgG (H+L) secondary antibody (Invitrogen, Cat #: A-11013) for detection. The binding signal was assessed by imaging the cells and quantifying the fluorescence intensity using Cytation (Biotek, VT).

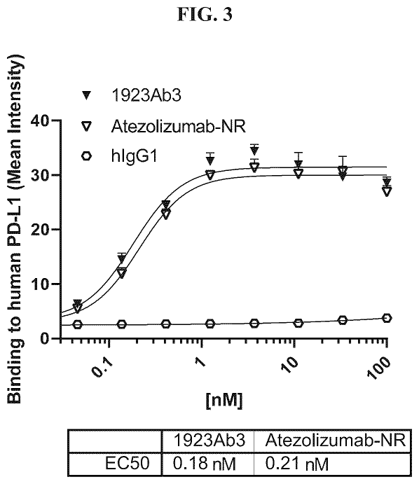

The binding result showed that 1923Ab3 exhibited strong binding to PD-L1 expressing on the cell surface. The binding affinity of 1923Ab3 is similar to the reference antibody, atezolizumab (Roche). The binding EC50 of 1923Ab3 and atezolizumab to human PD-L1 was determined as 0.18 nM and 0.21 nM, respectively (see FIG. 3).

### Example 4: Effect of PD-L1 Antibody on the PD-1/PD-L1 Interaction

The effect of the PD-L1 antibody on the interaction of PD-1 and PD-L1 was determined by a PD-1/PD-L1 inhibition bioassay developed by Promega (Madison, USA). This assay is a luciferase cell-based assay consisting of two genetically engineered cell lines: PD-1 effector cells, which are Jurkat T cells expressing human PD-1 on the cell surface and a stably integrated luciferase reporter driven by an NFAT response element (NFAT-RE), and artificial APC cells, which are CHO-K1 cells expressing cell surface human PD-L1 and an engineered cell surface protein designed to activate cognate TCRs in an antigen-independent manner. When these two cell types are co-cultured, the PD-1/PD-L1 interaction inhibits TCR signaling resulting in a reduction in luminescent signal. The addition of an antibody that blocks the PD-1/PD-L1 interaction removes the inhibitory signal, results in activation of TCR, and increases luminescence.

Artificial APC CHO-K1 cells (Promega, Cat #: J109A) were cultured according to the manufacturer's protocol using Ham's F-12K medium (ThermoFisher, Cat #: 21127022) containing 10% heat-inactivated Fetal Bovine Serum (Sigma, Cat #: 17H165). These artificial APC cells were plated in 384 well white TC treated plates (Corning, Cat #: 3570). The plates were incubated for 16 hours at 37° C., 5% CO₂. The supernatant was removed and serially diluted antibodies were added. PD-1 effector cells (Promega, Cat #: J115A) were culture according to the manufacturer's protocol using RPMI-1640 (ThermoFisher, Cat #: 11875-085) containing 10% heat-inactivated Fetal Bovine Serum. The effector cells were added to the white 384 well plates containing the artificial APC cells and antibodies. The plates were incubated for 6 hours at 37° C., 5% CO₂. After equilibration to room temperature, One-Glo luciferase reagent (Promega, Cat #: E6130) was added to each well. The plates were then incubated at room temperature for 5 minutes and luminescence was measured using a Synergy Neo2 plate reader (Biotek). The data was analyzed with GraphPad Prism software.

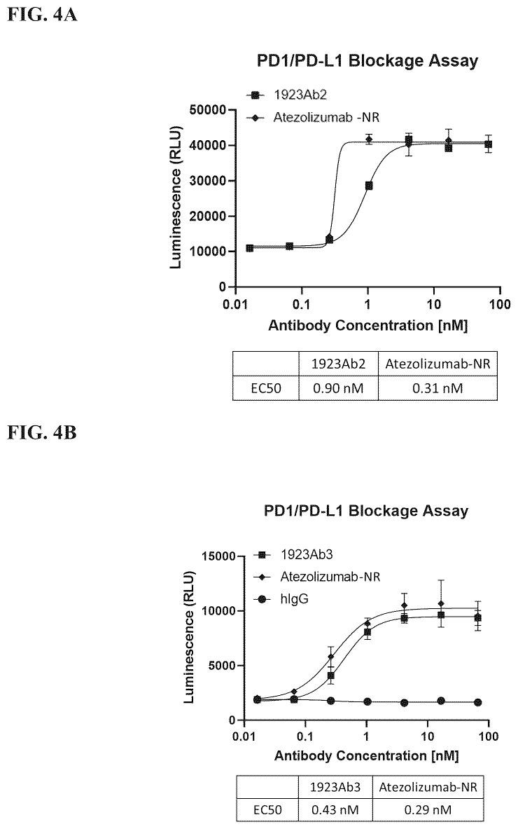

FIG. 4 shows that both 1923Ab2 and 1923Ab3 antibodies efficiently inhibited PD1/PD-L1 interaction which resulted in the restoration of luminescence signal in the assay. The IC50 of 1923Ab2, 1923Ab3 and atezolizumab to PD1/PD-L1 blockade was determined as 0.90 nM, 0.43 nM and 0.29-0.31 nM, respectively.

### Example 5: Generation of Binding Proteins that Bind CD137

Fully human anti-human CD137 antibodies were generated by immunizing human Ig transgenic mice, Trianni mice that express human antibody VH and VL genes (see, e.g., WO 2013/063391, TRIANNI® mice).

Immunization-TRIANNI mice described above were immunized by injection with the immunogens, which included HEK293 cells stably transfected with the human CD137 gene and recombinant human CD137 ECD protein. The TRIANI mice were immunized via intraperitoneally (IP), subcutaneously (SC), base of tail or footpad injections.

The immune response was monitored by retroorbital bleeds. The plasma was screened by ELISA, flow cytometry (FACS) or Imaging (as described below). Mice with sufficient anti-CD137 titers were used for fusions. Mice were boosted intraperitoneally, at the base of the tail or footpad with the immunogen before sacrifice and removal of the spleen and lymph nodes.

Selection of Mice Producing Anti-CD137 Antibodies—to select mice producing antibodies that bound CD137, sera from immunized mice were screened by ELISA, FACS or imaging for binding to human CD137 protein, cells expressing CD137 protein (HEK293T transfected with the CD137 gene) not the control cells that do not express CD137 (HEK293T cells).

For ELISA, briefly, an ELISA plate coated with recombinant human CD137 (R&D, catalog #: 9220-4B) was incubated with dilutions of serum from immunized mice for one hour at room temperature, the assay plate was washed, and specific antibody binding was detected with HRP-labeled anti-mouse IgG antibody (Jackson ImmunoResearch, catalog number: 109-035-088) after one hour incubation at room temperature, washed, and followed by ABTS substrate (Moss, catalog number: ABTS-1000) incubation for 30 minutes at room temperature. The plate was read using an ELISA plate reader (Biotek).

For FACS, briefly, CD137-HEK293T cells or parental HEK293T cells were incubated with dilutions of serum from immunized mice for 2 hours at 4° C. Cells were fixed with 2% PFA (Alfa Aesar, catalog number: J61899) for 15 minutes at 4° C. and then washed. Specific antibody binding was detected with Alexa 647 labeled goat anti-mouse IgG antibody (Thermofisher Scientific, catalog number: A-21445) after one-hour incubation at 4° C. Flow cytometric analyses were performed on a flow cytometry instrument (Intellicyte, IQue plus, Sartorius).

In addition, mice serum was tested by imaging. Briefly, CD137-HEK293T cells were incubated with dilutions of serum from immunized mice. Cells were washed, fixed with paraformaldehyde, washed, specific antibody binding was detected with secondary Alexa488 goat anti-mouse antibody and Hoechst (Invitrogen). Plates were scanned and analyzed on an imaging machine (Cytation 5, Biotek).

Generation of Hybridomas Producing Antibodies to CD137—to generate hybridomas producing human antibodies of the invention, splenocytes and lymph node cells were isolated from an immunized mouse and fused to an appropriate immortalized cell line, such as a mouse myeloma cell line. The resulting hybridomas were screened for the production of antigen-specific antibodies. For example, single cell suspensions of splenocytes, lymph node cells from immunized mice were fused to an equal number of Sp2/0 non-secreting mouse IgG myeloma cells (ATCC, CRL 1581) by electrofusion. Cells were plated in flat bottom 96-well tissue culture plates, followed by about one week of incubation in selection medium (HAT medium), then switched to hybridoma culture media. Approximately 10-14 days after cell plating, supernatants from individual wells were screened by Imaging or FACS as described above. The antibody secreting-hybridomas were transferred to 24-well plates, screened again, and if still positive for anti-CD137, the positive hybridomas were subcloned by sorting using a single cell sorter. The subclones were screened again by Imaging or FACS as described above. The stable subclones were then cultured in vitro to generate small amounts of antibodies for purification and characterization.

### Example 6: Binding Affinity of Recombinant Anti-CD137 Antibodies to Human, Mouse and Cynomolgus CD137

Analysis of binding affinity of anti-CD137 mAb with immunofluorescence imaging assay was performed using HEK293T cells stably transfected with human, mouse or cynomolgus CD137 expression construct. These cell lines express specie specific form of CD137 protein on the cell surface. The cells were plated in complete media containing DMEM with 10% FBS, then incubated overnight at 37° C. Cells were stained with a serial dilution of anti-CD137 at 4° C. for 2 hours followed by fixing cells for 15 minutes at room temperature. The fixed cells were washed with PBS three times following by staining at room temperature for 1 hour with Alexa Fluor® 488 Goat Anti-Human IgG (H+L) secondary antibody (Invitrogen, Cat #: A-11013) for detection. The binding signal was assessed by imaging the cells and quantifying the fluorescence intensity using a Biotek Cytation.

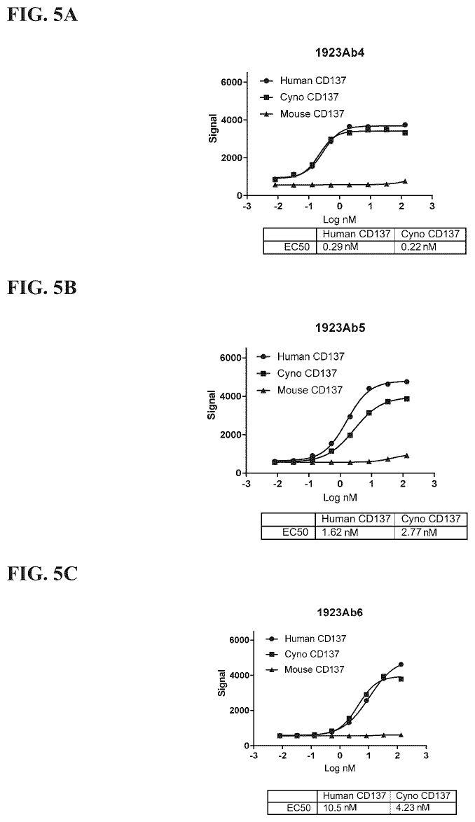

The result shows that 1923Ab4 (FIG. 5A), 1923Ab5 (FIG. 5B) and 1923Ab6 (FIG. 5C) antibodies efficiently bound to human and cynomolgus CD137 on the cell surface. However, none of them shows binding to mouse CD137. The binding EC50 of 1923Ab4, 1923Ab5 and 1923Ab6 to human CD137 was determined as 0.29 nM, 1.62 nM and 10.5 nM respectively. The binding EC50 of 1923Ab4, 1923Ab5 and 1923Ab6 to cynomolgus CD137 was determined as 0.22 nM, 2.77 nM and 4.23 nM respectively.

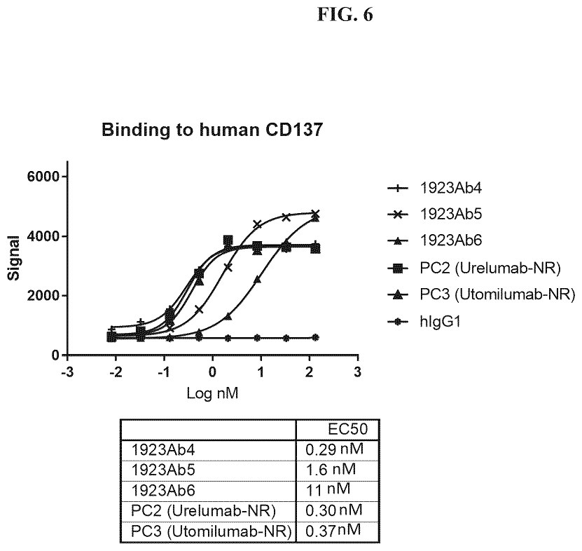

The experiment was also performed to compare the binding affinity of 1923Ab4, 1923Ab5 and 1923Ab6 antibodies to reference antibodies including PC2 Urelumab-NR (BMS) and PC3 Utomilumab-NR (Pfizer). 1923Ab4 antibody exhibited a similar binding affinity of Urelumab-NR and Utomilumab-NR to human CD137 (FIG. 6). 1923Ab5 and 1923Ab6 antibodies showed weaker binding compared to reference antibodies.

### Example 7: CD137 Ligand Competition

To assess the ability of the disclosed anti-CD137 antibodies to block CD137 ligand binding to CD137, the disclosed antibodies, 1923Ab4, 1923Ab5 and 1923Ab6, and two reference antibodies, PC2 Urelumab-NR and PC3 Utomilumab-NR, were tested by biolayer interferometry (Gator Bio, CA). Urelumab-NR and Utomilumab-NR have been reported to be non-ligand and ligand blocking antibodies, respectively.

Briefly, streptavidin probes (Probe Life, Catalog number: PL168-1600002) were first loaded into 96-well plates containing the assay buffer (PBS containing 0.02% Tween20 and 0.05% sodium azide) for 30 seconds (baseline step). The probes were then loaded into 96-wells containing CD137L His-Avi-Tag protein (BPS Bioscience, catalog number: 100238; 10 ug/ml) for 180 seconds (loading step, to capture biotin-CD137L) followed by 30 second baseline step. After that, the CD137L loaded probes were allowed to bind CD137 protein (R&D, catalog number: 9220-4B; 10 ug/ml) for 180 seconds followed by 180 seconds association with disclosed antibodies or reference antibodies at a concentration of 10 ug/ml.

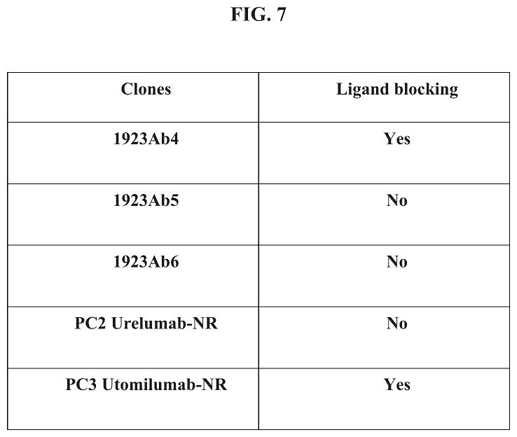

Data was processed using software provided by the manufacturer (Gator Bio, CA). 1923Ab5, 1923Ab6 and Urelumab-NR bound to the complex of CD137/CD137L loaded on the probe. However, 1923Ab4 and Utomilumab-NR showed no binding to the complex of CD137/CD137L loaded on the probe. These results implied that 1923Ab4 and Utomilumab-NR have ligand blocking activity. 1923Ab5, 1923Ab6 and Urelumab-NR bound to a region of CD137 which is not located in the ligand binding site (FIG. 7).

### Example 8: Cross-Linking Dependent Agonistic Activity of Anti-CD137 Antibodies in NFκB Luciferase Reporter Assay

Agonist activity of antibodies was evaluated using NFκB luciferase reporter assay. 293T cells stably transfected with human CD137 expression plasmid and NFκB luciferase reporter plasmid were used to measure the agonistic activity of anti-CD137 antibodies. These reporter cells (HEK-CD137 reporter cells) were stimulated with either anti-CD137 antibody or mixture containing tested anti-CD137 antibody and 3:1 ratio of cross-linking antibody (anti-human Fcγ fragment specific (Jackson ImmunoResearch Lab, Cat #: 109-005-098) and incubated for 16 hours at 37° C. with 5% CO₂. ONE-Glo™ luciferase reagent (Promega, Cat #: E6130) was added and the plate was incubated at room temperature for 10 minutes. The luminescence signal was measured by Synergy Neo2 plate reader (Biotek) and data was analyzed by GraphPad Prism.

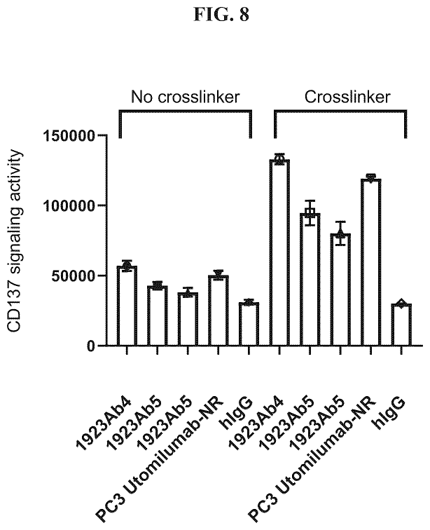

PC3 Utomilumab-NR, which was used as a control antibody, has been reported to be a cross-linking dependent agonistic antibody to CD137 signaling. As shown in FIG. 8, compared to isotype control, all tested antibodies showed very minimal agonistic activity in the absence of cross-linking antibodies. However, all tested antibodies, except isotype control, cross-linked by anti-human Fcγ strongly activated NFκB luciferase gene expression. The result demonstrates that agonistic activity of 1923Ab4, 1923Ab5 and 1923Ab6 antibodies is cross-linking dependent.

### Example 9: Cross Linking Dependent Agonistic Activity of Anti-CD137 Antibodies in Human Primary T Cell Activation Assay

Agonist activity of antibodies was further confirmed in the T cell activation assay. Human PBMCs were prepared from a healthy donor. These human PBMCs were cultured at density of 1×10⁶ cells/mL in RPMI1640 media supplemented with 10% FBS and 0.5 ug/ml of mouse anti-hCD3 clone OKT3 (Biolegend, Cat #: 317325). Either anti-CD137 antibody or mixture containing tested anti-CD137 antibody and 3:1 ratio of cross-linking antibody was added to stimulate T cells. The plate was incubated for 3 days at 37° C. with 5% CO₂. After 72 hours of incubation, the supernatant was used to measure the secreted IFNγ by AlphaLISA (PerkinElmer, Cat #: AL217C/F) using protocols according to the manufacture's instruction.

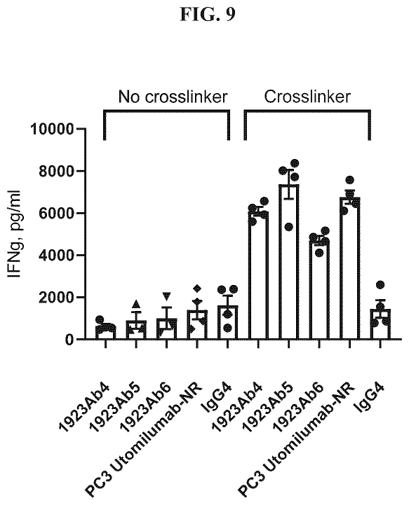

PC3 Utomilumab-NR, which was used as a positive control antibody, has been reported to be a cross-linking dependent agonistic antibody to T cell activation. As shown in FIG. 9, compared to isotype control, PBMCs treated with the disclosed antibodies did not increase the production of IFNγ in the absence of cross-linking antibodies. However, all tested antibodies, except isotype control, cross-linked by anti-human Fcγ strongly stimulated the production of IFNγ. This result demonstrates that 1923Ab4, 1923Ab5 and 1923Ab6 antibodies activated T cells in a cross-linking dependent manner.

### Example 10: Identification of Binding Epitope Regions of 1923Ab4 on Human CD137

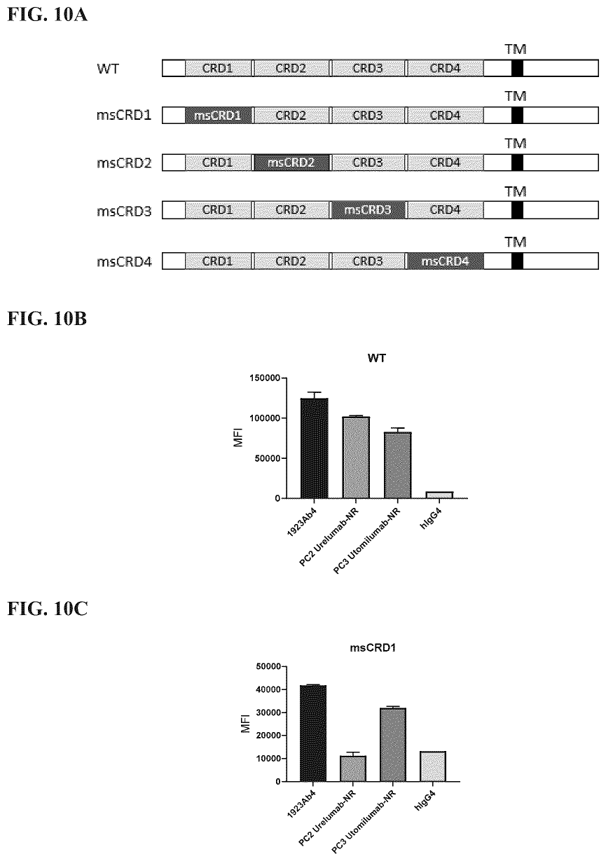

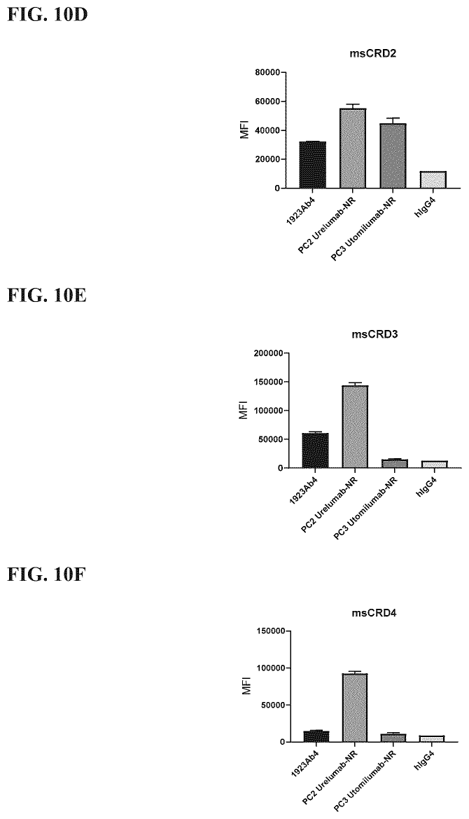

Extracellular region of CD137 contains four cysteine-rich domains (CRD1-4), which are conserved among species. To identify which CRD domain is required for binding to 1923Ab4 antibody, human/mouse hybrid CD137 expression constructs were made by swapping individual human CRD domain with mouse counterpart (FIG. 10A). Example 6 shows that 1923Ab4 is bound to human CD137 but not mouse CD137 on the cell surface.

Analysis of binding of anti-CD137 1923Ab4 to HEK293T cells transiently transfected with either human CD137 (WT) or human/mouse hybrid CD137 expression constructs (msCRD1-4) was performed by flow cytometry based binding assay. Cells were stained with 1923Ab4 at 4° C. for 2 hours followed by fixing cells for 15 minutes at room temperature. The fixed cells were washed with PBS three times following by staining at room temperature for 1 hour with Alexa Fluor® 488 Goat Anti-Human IgG antibody (Invitrogen, Cat #: A-11013) for detection. The binding signal was assessed by quantifying the fluorescence intensity using iQue Screener PLUS (Sartorius, MI).

Both PC2 Urelumab-NR and PC3 Utomilumab-NR bind to human CD137 but not to mouse CD137. Binding of PC2 and PC3 to human CD137 was through CRD1 and CRD3/4 domains respectively. In this binding experiment, PC2 lost binding to CD137 only when the human CRD1 domain was swapped to the mouse CRD1 domain, whereas PC3 lost binding to CD137 when either human CRD3 or CRD4 domain was swapped to mouse counterpart (FIG. 10B-F). The disclosed antibody 1923Ab4 shows reduced binding to the cells transfected with msCRD2, msCRD3 and msCRD4 expression constructs (FIG. 10B-F). This result suggests that 1923Ab4 binds to human CD137 through CRD2, CRD3 and CRD4 regions.

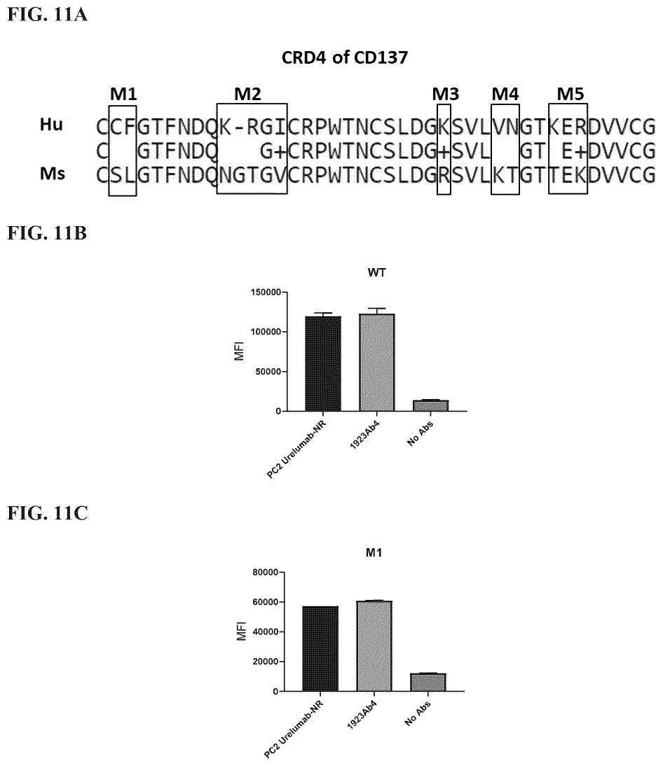

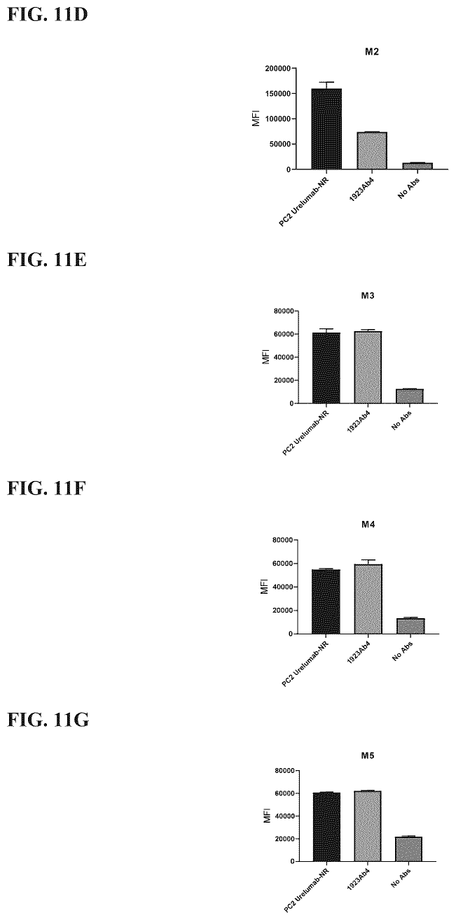

In FIG. 11A, sequence alignment of human and mouse CRD4 regions revealed 5 distinct differences. Additional expression constructs (M1 to M5) were made by changing the human amino acid sequence into the mouse one. For example, human “CF” sequence in M1 region was mutated to mouse “SL” sequence by site-directed mutagenesis.

Analysis of binding of anti-CD137 1923Ab4 to HEK293T cells transiently transfected with either human CD137 (WT) or mutated CD137 expression constructs (M1-M5) was performed by flow cytometry-based binding assay. Cells were stained with 1923Ab4 at 4° C. for 2 hours followed by fixing cells for 15 minutes at room temperature. The fixed cells were washed with PBS three times followed by staining at room temperature for 1 hour with Alexa Fluor® 488 Goat Anti-Human IgG antibody (Invitrogen, Cat #: A-11013) for detection. The binding signal was assessed by quantifying the fluorescence intensity using iQue Screener PLUS (Sartorius, MI).

In FIG. 11D, PC2 Urelumab-NR was used as expression control, its binding epitope was mapped to CRD1 domain (Chin, S M et al, Nat Commun, 2018 Nov. 8; 9(1):4679). Changing the human amino acid sequence of “KRGI” (SEQ ID NO: 81) to the mouse amino sequence of “NGTGV” (SEQ ID NO: 77) in the M2 region of CRD4 domain of CD137 greatly reduced its binding to the disclosed antibody 1923Ab4. Mutagenesis on M1, M3, M4 and M5 did not alter the binding activity of 1923Ab4 to CD137 protein expressed on the cell surface (FIGS. 11B, 11C & 11E-G).

### Example 11: Epitope Mapping of 1923Ab4 on Human CD137 Using HDX Mass Spectrometry

Using domain swapping and mutagenesis study, 1923Ab4 was showed bind to human CD137 through CRD2, CRD3 and CRD4 domains (example 10). To further identify the binding sites of 1923Ab4 to human CD137, Hydrogen deuterium exchange (HDX) mass spectrometry was employed. The region of human CD137 bound by the antibody, which is defined as epitope, is protected from hydrogen/deuterium exchange. The mass difference of digested peptides was revealed by LC-MS.

Recombinant CD137 was first incubated in deuterium oxide either alone or in complex with the 1923Ab4 antibody. The deuterium exchange was carried at 20° C. for 0 s, 15 s, 60 s, 600 s, or 3600 s. The exchange reaction was quenched by low pH, and the proteins were digested with pepsin/prolyl endopeptidase/XIII. The deuterium levels at the digested peptides of CD137 were monitored from the mass shift on LC-MS.

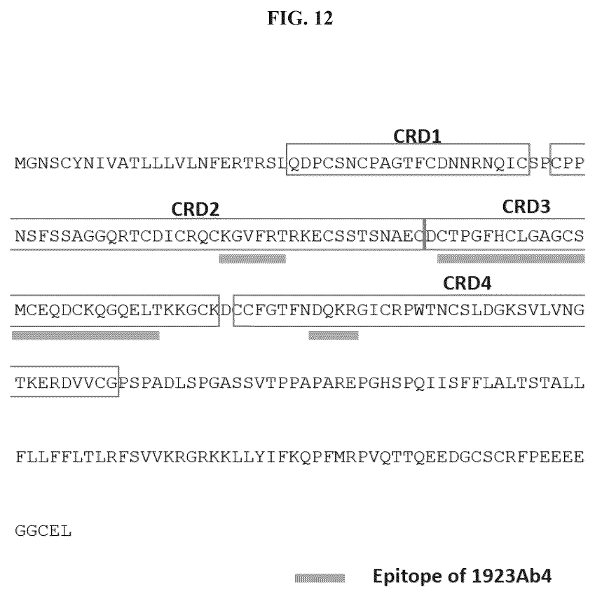

The recombinant CD137 showed a significant reduction in deuterium uptakes upon binding to the 1923Ab4 antibody at sequences AA46-51 (KGVFRT; SEQ ID NO: 78), AA65-90 (CTPGFHCLGAGCSMCEQDCKQGQELT; SEQ ID NO: 79) and AA104-107 (DQKR; SEQ ID NO: 80) (regions depicted as shaded bars in FIG. 12). This data is consistent with the domain swapping experiments illustrated in Example 10. The recombinant CD137 showed a significant reduction in deuterium uptakes upon binding to the 1923Ab4 antibody at sequences AA46-51 (KGVFRT; SEQ ID NO: 78), AA65-90 (CTPGFHCLGAGCSMCEQDCKQGQELT; SEQ ID NO: 79) and AA104-107 (DQKR; SEQ ID NO: 80). The epitope mapping result depicted as shaded bars was summarized in FIG. 12. This data is consistent with the domain swapping experiments illustrated in Example 10

### Example 12: Preparation of scFvs that Bind Against PD-L1 or CD137

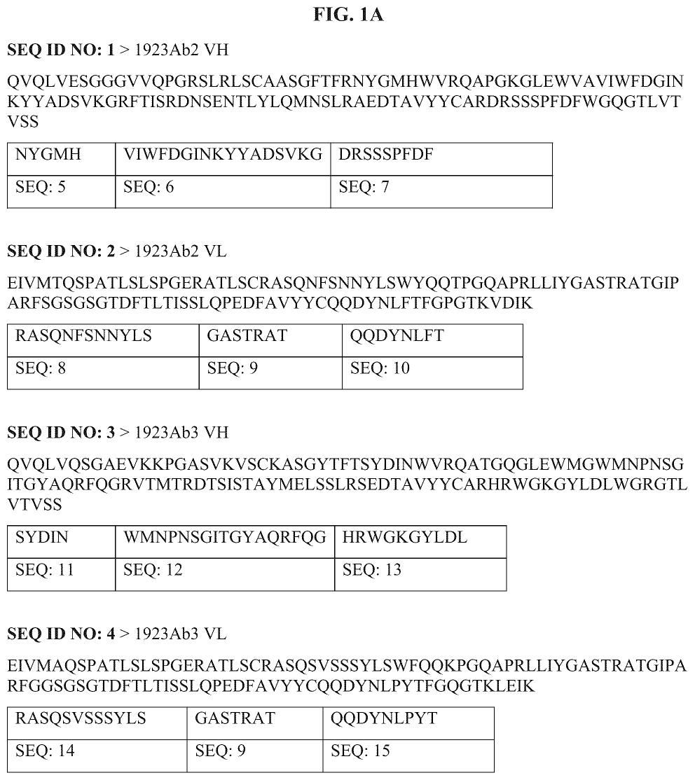

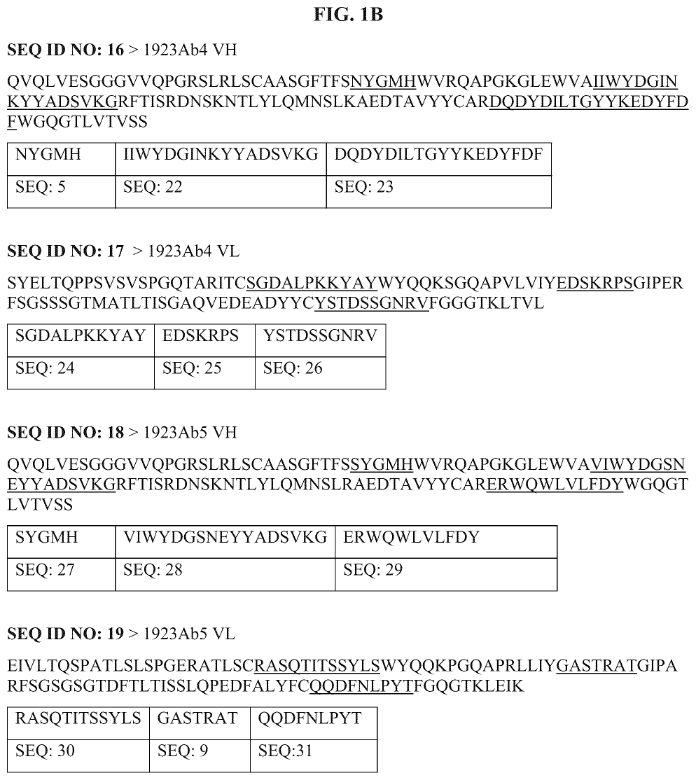

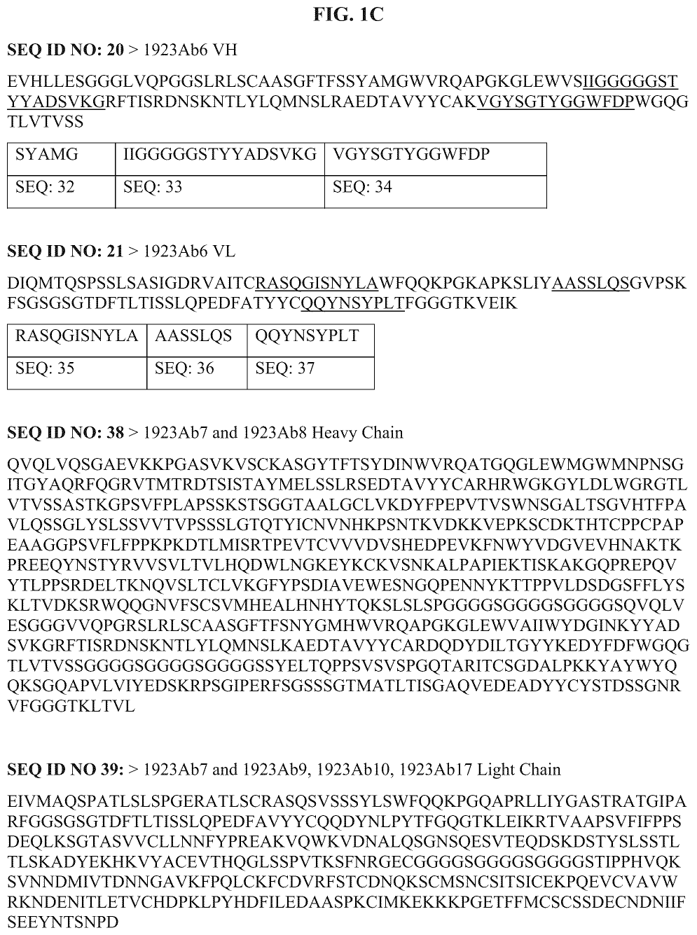

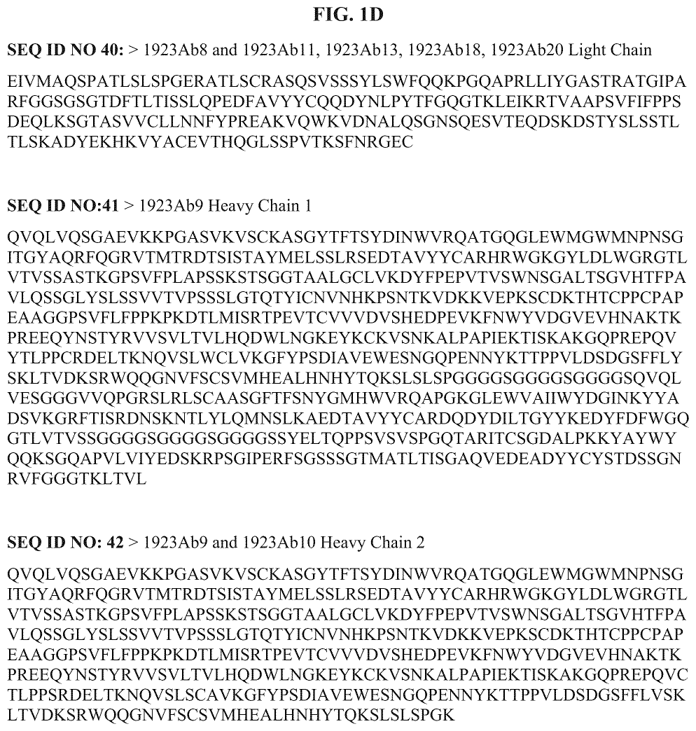

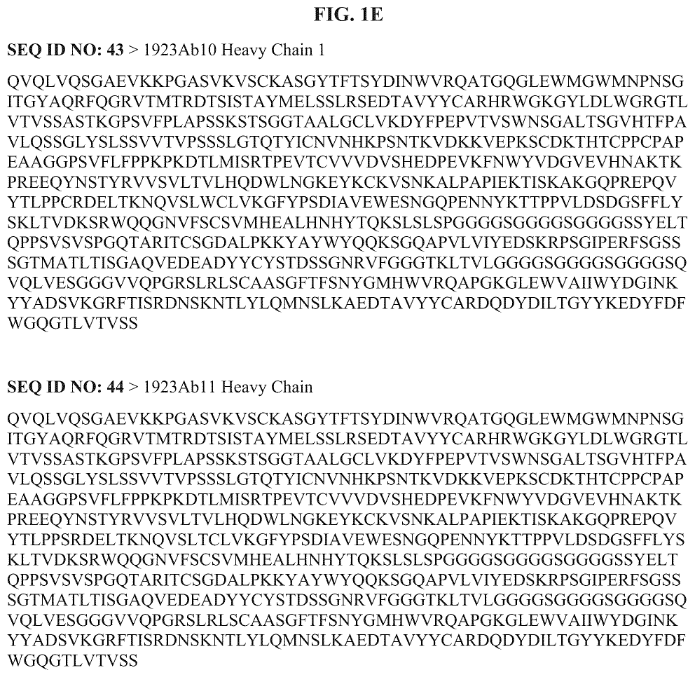

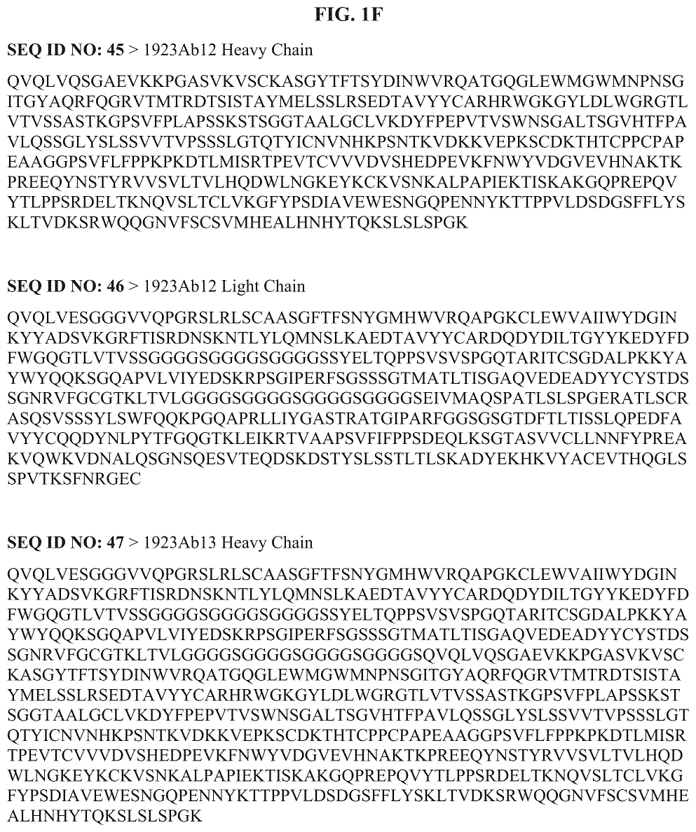

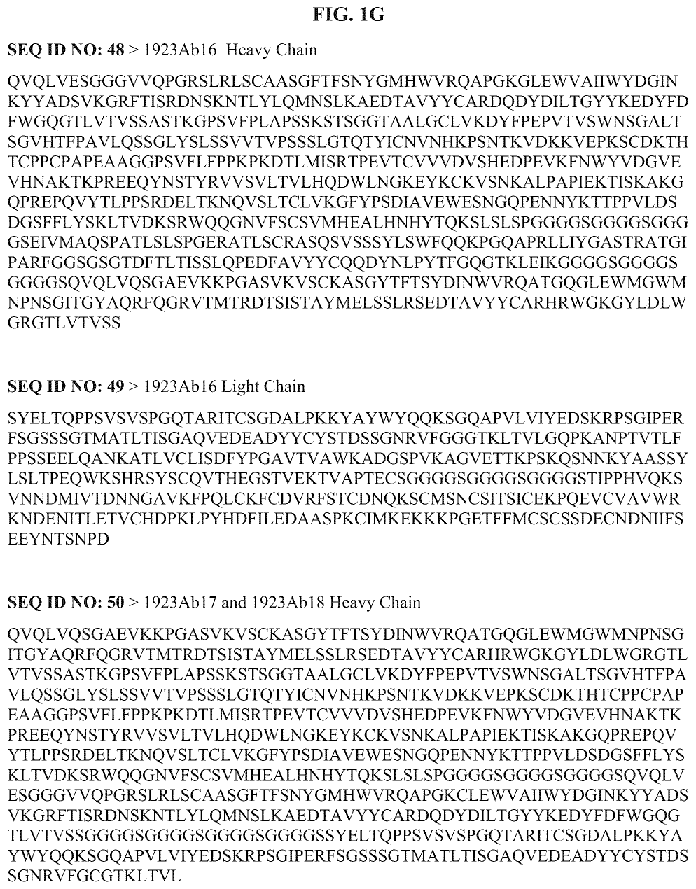

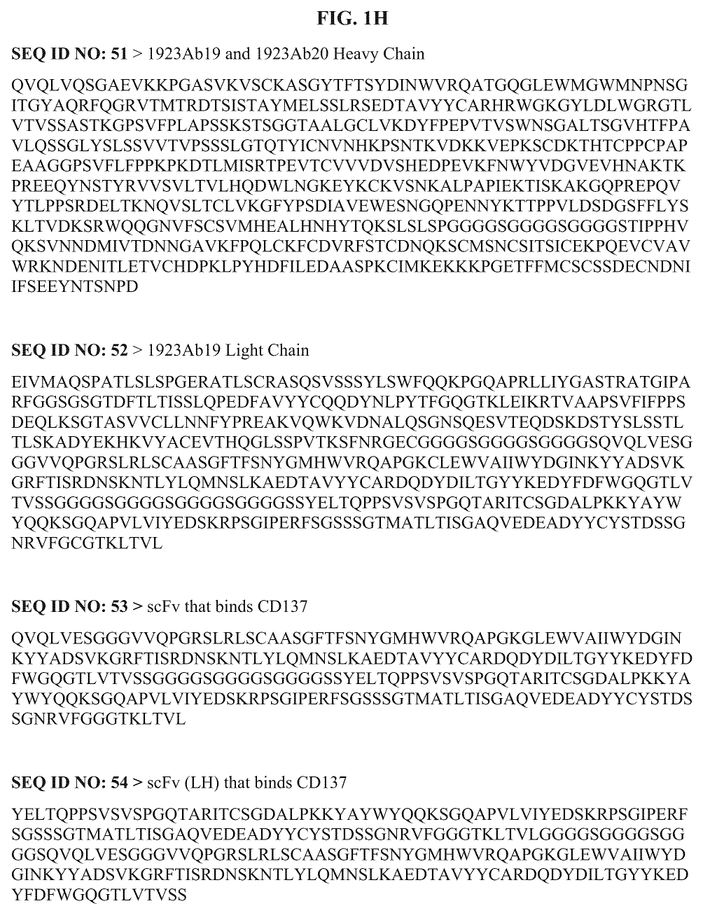

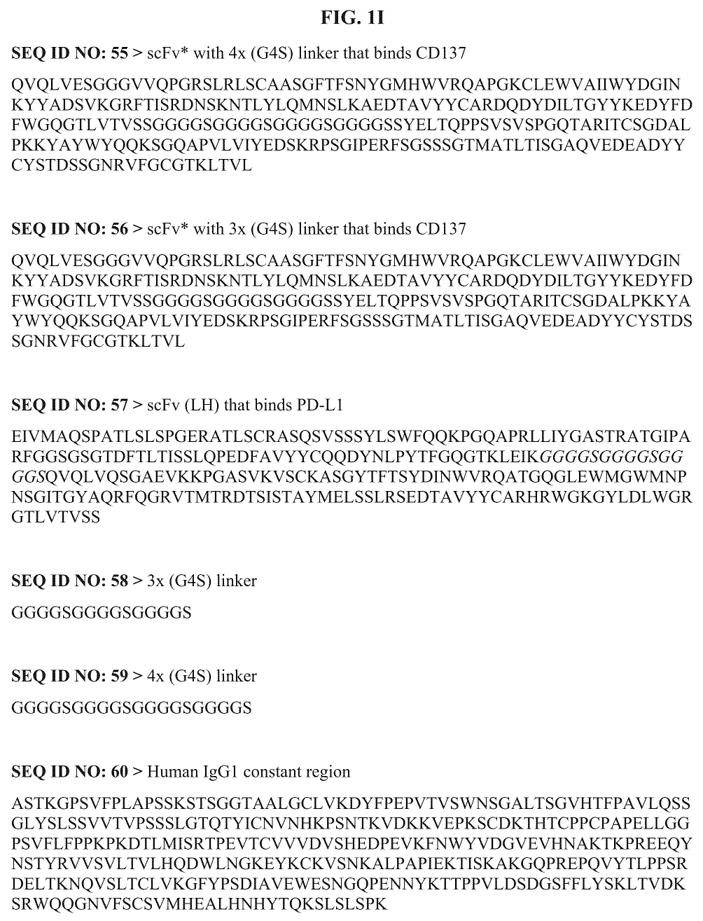

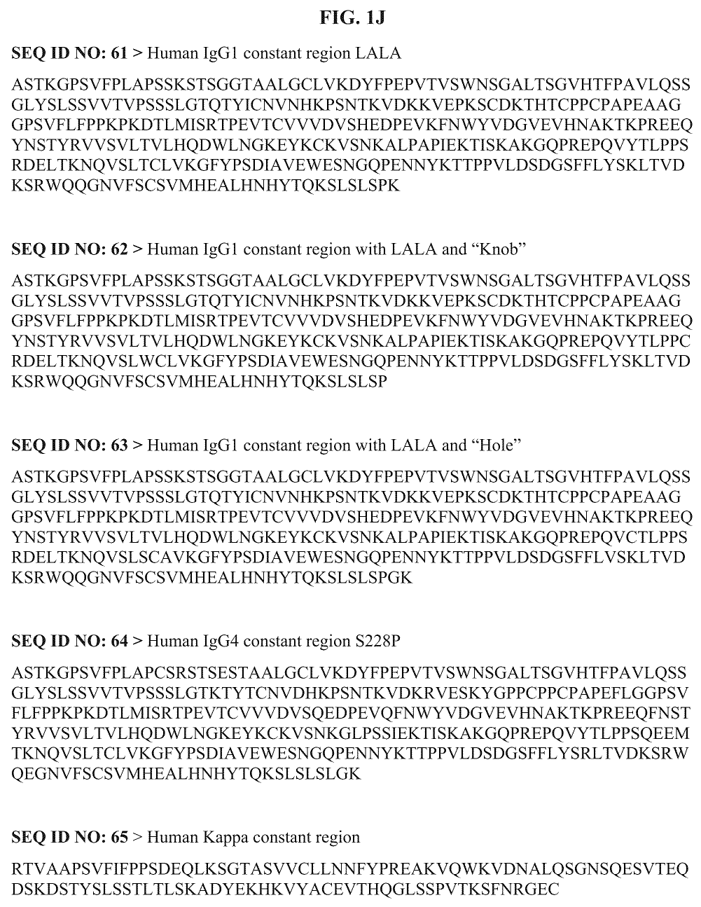

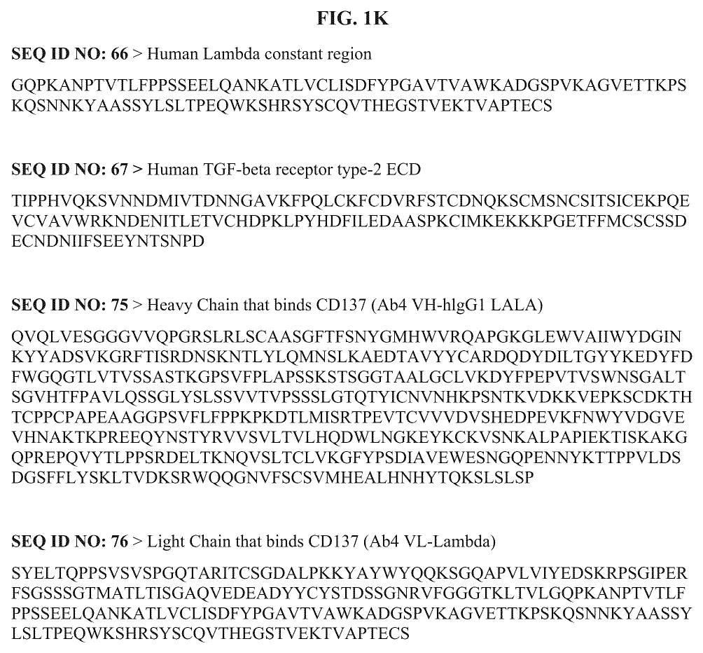

scFvs that bind PD-L1 with a structure of (N)-VL-Linker-VH-(C) or (N)-VH-Linker-VL-(C) were prepared using the variable regions of the full human monoclonal antibodies against PD-L1 shown in FIG. 1A.

scFvs that bind CD137 with a structure of (N)-VL-Linker-VH-(C) or (N)-VH-Linker-VL-(C) were prepared using the variable regions of the full human monoclonal antibodies against CD137 shown in FIGS. 1B and 1C, where the amino acid residue “G” at the position of 44 of a heavy chain variable region could be substituted with “C”, and the amino acid residue “G” at the position of 100 of a light chain variable region could be substituted with “C”. Such amino acid substitution from “G” to “C” in scFv could improve the stabilities of scFv as one target-specific moiety of bispecific antibodies or tri-specific antibodies.

### Example 13: Molecular Design and Production of a PD-L1/CD137 Bispecific

As a representative example of a binding protein that binds PD-L1, a symmetrical bispecific (PD-L1×CD137) characterized by the molecular format depicted in FIG. 13J comprising the subunit/components summarized in FIGS. 13 and 14 was prepared:

### 1923Ab18

1. Heavy Chain: SEQ ID NO: 50 comprising the components: heavy chain of anti-PD-L1 antibody, linker and anti-CD137 scFv (VH-VL with CC) (N→C); and
2. Light Chain: SEQ ID NO: 40 comprising an anti-PD-L1 antibody light chain.

A DNA segment 1 having a polynucleotide sequence encoding the heavy chain component of 1923Ab18 (SEQ ID NO:50) was inserted into an expression vector, and a DNA segment 2 having a polynucleotide sequence encoding the light chain of 1923Ab18 (SEQ ID NO:40) was inserted in the expression vector.

The constructed expression vectors were transiently expressed in Expi293 cells (ThermoFisher), cultured in Expi293 Expression medium under the condition of 37° C. for 5 days in a CO₂ incubator. The bispecific antibody was purified from the cell culture supernatant by recombinant protein A affinity chromatography (Hitrap Mabselect SuRe, GE) and second step purification by Ion exchange chromatography or gel filtration chromatography if necessary. SDS-PAGE (BiRad), size exclusion HPLC (Agilent, 1100 series) analysis with SE-HPLC column (TOSO, G3000SWXL) and CE-SDS (SCIEX, PA800 Plus) were performed to detect and confirm the size and purity of bispecific antibody. Purified proteins were buffer-exchanged into the desired buffer and concentrated by ultrafiltration using an Amicon Ultra 15 30K device, and protein concentrations were estimated using dropsense (Unchained Lab). The transient transfection could be used in a two-vector system or with a one-vector system that contains both heavy and light chain components in one single vector. Alternatively, the bispecific antibody could be purified from the supernatant of stable CHO expression cell lines.

### Example 14: Molecular Design and Production of an Asymmetric PD-L1/TGFβ/CD137 Trispecific

As a representative example of an asymmetric binding protein that binds PD-L1, a trispecific (PD-L1×CD137×TGFβRII) characterized by the molecular format depicted in FIG. 13C comprising the subunit/components summarized in FIGS. 13 and 14 was prepared:

### 1923Ab9

1. Heavy chain (knob): SEQ ID NO: 41 comprising the components: heavy chain of anti-PD-L1 antibody, linker and anti-CD137 scFv (VH-VL with CC) (N→C);
2. Heavy chain (hole): SEQ ID NO: 42 comprising heavy chain of anti-PD-L1 antibody components (N→C); and
3. Light chain: SEQ ID NO: 39 comprising an anti-PD-L1 antibody light chain.

A DNA segment 1 having a polynucleotide sequence encoding the heavy chain (knob) component of the 1923Ab9 (SEQ ID NO:41) was inserted into the expression vector, a DNA segment 2 having a polynucleotide sequence encoding the heavy chain (hole) component of the 1923Ab9 (SEQ ID NO: 42) was insert into the expression vector, and a DNA segment 3 having a polynucleotide sequence encoding the light chain of the 1923Ab9 (SEQ ID NO: 39) was inserted in the expression vector.

The constructed expression vectors were transiently expressed in Expi293 cells (ThermoFisher), cultured in Expi293 Expression medium under the condition of 37° C. for 5 days in a CO₂ incubator. The bispecific antibody was purified from the cell culture supernatant by recombinant protein A affinity chromatography (Hitrap Mabselect SuRe, GE) and second step purification by Ion exchange chromatography or gel filtration chromatography if necessary. SDS-PAGE (BioRad), size exclusion HPLC (Agilent, 1100 series) analysis with SE-HPLC column (TOSO, G3000SWXL) and CE-SDS (SCIEX, PA800 Plus) were performed to detect and confirm the size and purity of trispecific antibodies. Purified proteins were buffer-exchanged into the desired buffer and concentrated by ultrafiltration using an Amicon Ultra 15 30K device, and protein concentrations were estimated using dropsense (Unchained Lab). The transient transfection could be used in a three-vector system or with the one-vector system that contains both heavy and light chain components in one single vector. Alternatively, the bispecific antibody could be purified from the supernatant of stable CHO expression cell lines.

### Example 15: Molecular Design and Production of a PD-L1/TGFβ/CD137 Trispecific

As a representative example of a symmetric binding protein that binds PD-L1, a trispecific (PD-L1×CD137×TGFβRII, 1923Ab17) characterized by the molecular format depicted in FIG. 13I comprising the subunit/components summarized in FIGS. 13 and 14 was prepared:

### 1923Ab17

1. Heavy Chain: SEQ ID NO: 50 comprising the components, heavy chain of anti-PD-L1 antibody, linker and anti-CD137 scFv (VH-VL with CC) (N→C); and
2. Light chain: SEQ ID NO:39 comprising the components, light chain of anti-PD-L1 antibody, linker and TGFβRII ECD.

A DNA segment (1) having a polynucleotide sequence encoding the heavy chain component of the 1923Ab17 (SEQ ID NO: 50) was insert into the expression vector, and a DNA segment (2) having a polynucleotide sequence encoding the light chain component of the 1923Ab17 (SEQ ID NO: 39) was inserted in the expression vector.

The constructed expression vectors were transiently expressed in Expi293 cells (ThermoFisher), cultured in Expi293 Expression medium under the condition of 37° C. for 5 days in a CO₂ incubator. The tri-specific antibody was purified from the cell culture supernatant by recombinant protein A affinity chromatography (Hitrap Mabselect SuRe, GE) and second step purification by Ion exchange chromatography or gel filtration chromatography if necessary. SDS-PAGE (BioRad), size exclusion HPLC (Agilent, 1100 series) analysis with SE-HPLC column (TOSO, G3000SWXL) and CE-SDS (SCIEX, PA800 Plus) were performed to detect and confirm the size and purity of tri-specific antibody. Purified proteins were buffer-exchanged into the desired buffer and concentrated by ultrafiltration using an Amicon Ultra 15 30K device, and protein concentrations were estimated using dropsense (Unchained Lab). The transient transfection could be used in a two-vector system or with a one-vector system that contains both heavy and light chain components in one single vector. Alternatively, the bispecific antibody could be purified from the supernatant of stable CHO expression cell lines.

### Example 16: Characterization of Bispecifics and Trispecifics that Bind PD-L1: Binding to CD137

Bispecific and trispecific antibodies were generated, produced, and purified as described in Examples 13 through 15. To examine the binding activity of these antibodies to CD137, an immunofluorescence imaging assay was performed using HEK293T cells stably transfected with a human CD137 expression construct. This cell line expressed human CD137 protein on the cell surface. The cells were plated in complete media containing DMEM with 10% FBS, then incubated overnight at 37° C. Cells were stained with these antibodies at 4° C. for 2 hours followed by fixing cells for 15 minutes at room temperature. The fixed cells were washed with PBS three times following by staining at room temperature for 1 hour with Alexa Fluor® 488 Goat Anti-Human IgG (H+L) secondary antibody (Invitrogen, Cat #: A-11013) for detection. The binding signal was assessed by imaging the cells and quantifying the fluorescence intensity using Cytation 5 (Biotek, VT).

In FIG. 16A, all the disclosed bispecific and trispecific antibodies including 1923Ab7, 1923Ab8, 1923Ab9, 1923Ab10 and 1923Ab11 bound similarly to human CD137 on the cell surface compared to 1923Ab4. This result indicates that the ScFv format of anti-CD137 retained binding activity to CD137.

We also generated a trispecific antibody using 1923Ab3 as ScFv format. In FIG. 16B, both 1923Ab7 and 1923Ab16 bound similar to human PD-L1 on the cell surface measured by flow cytometry. This result indicates that both 1923Ab3 and 1923Ab4 can be used as ScFv format in the disclosed bispecific and trispecific antibodies.

### Example 17: Characterization of Bispecifics and Trispecifics that Bind PD-L1: CD137 Signaling

These antibodies were further evaluated by measuring agonist activity to CD137 signaling using NFκB luciferase reporter assay. 293T cells stably transfected with human CD137 expression plasmid and NFκB luciferase reporter plasmid were used as reporter cells. These reporter cells were co-cultured with 293T cells expressing PD-L1 and stimulated with antibodies, and incubated for 16 hours at 37° C. with 5% CO₂. ONE-Glo™ luciferase reagent (Promega, Cat #: E6130) was added and the plate was incubated at room temperature for 10 minutes. The luminescence signal was measured by Synergy Neo2 plate reader (Biotek, VT) and data was analyzed by GraphPad Prism. Activation of CD137 signaling resulted in an increase of luminescence signal.

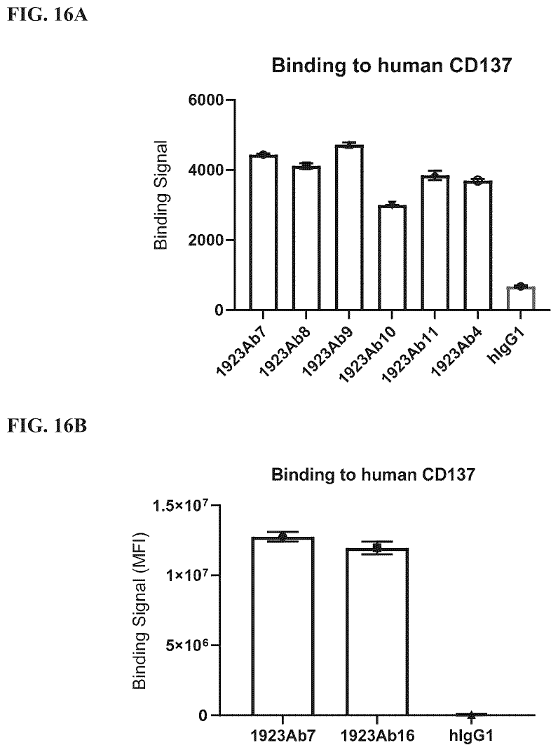

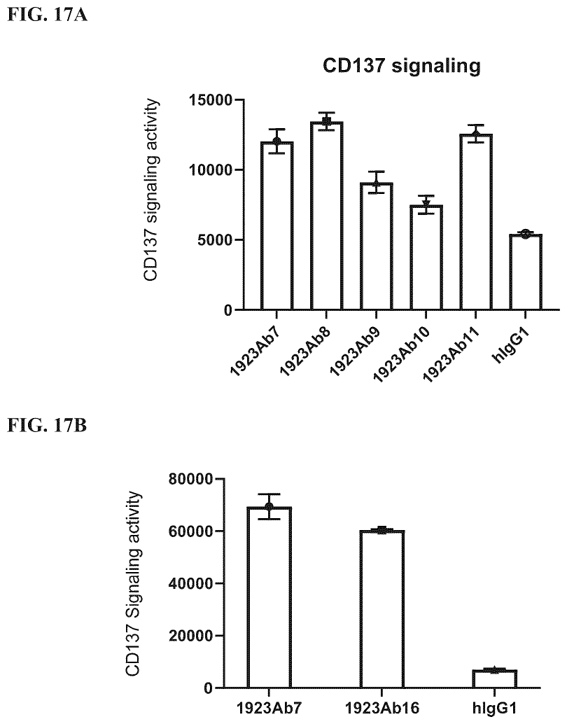

In FIG. 17A, all the disclosed antibodies including 1923Ab7, 1923Ab8, 1923Ab9, 1923Ab10 and 1923Ab11 activated CD137 signaling compared to isotype control antibody. 1923Ab7, 1923Ab8 and 1923Ab11 showed stronger agonistic activity compared to 1923Ab9 and 1923Ab10 (FIG. 17A). The result demonstrates that bivalent binding of anti-CD137 induced stronger CD137 signaling than the monovalent binding of anti-CD137.

We also generated a trispecific antibody using 1923Ab3 as ScFv format. The agonistic activity of these antibodies to CD137 signaling was also compared using Jurkat T CD137 reporter cells, expressing the recombinant CD137 and the NFκB luciferase reporter. In brief, the Jurkat T NFkB report cell line was used to measure the activity of CD137 signaling and a PD-L1 expressing HEK293T cell was used as a target cell to provide PD-L1. In FIG. 17B, both 1923Ab7 and 1923Ab16 showed similar agonistic activity to CD137 signaling measured by the Jurkat T CD137 reporter cells. 1923Ab16 is effectively bound to human PD-L1 to activate CD137 signaling.

### Example 18: Characterization of Bispecifics and Trispecifics—PD-L1 Dependent Activation of CD137 Signaling

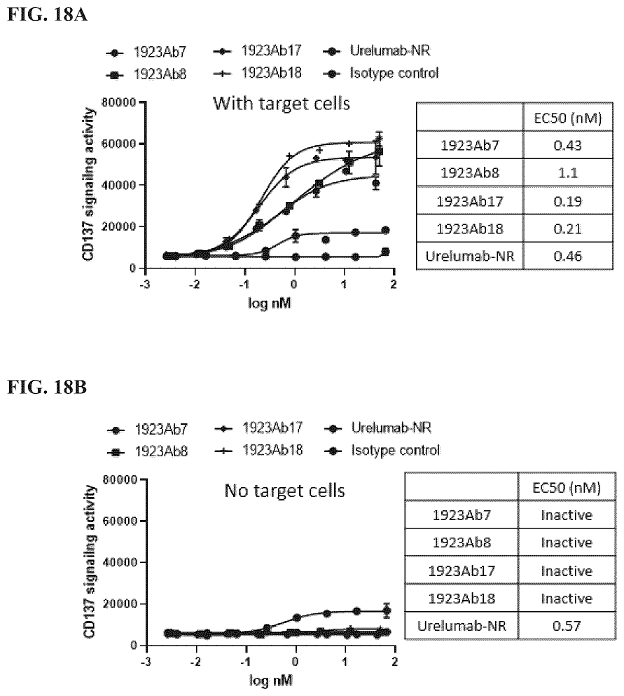

The disclosed antibodies were evaluated for their ability to induce PD-L1 dependent CD137 agonism. FIG. 18 demonstrates the ability of 1923Ab7, 1923Ab8, 1923Ab17 and 1923Ab18 to induce CD137 signaling using the Jurkat T CD137 reporter cells in the presence (18A) or absence (18B) of the target cell. In brief, a CD137 expressing Jurkat T NFkB report cell line was used to measure the activity of CD137 signaling and a PD-L1 expressing HEK293T cell was used as target cell to provide PD-L1. When the reporter cells were co-cultured with the target cells, the disclosed antibodies including 1923Ab7, 1923Ab8, 1923Ab17, 1923Ab18, and Urelumab-NR showed activation of CD137 signaling (FIG. 18A). However, none of the disclosed antibodies induced CD137 signaling in the absence of target cells except Urelumab-NR (FIG. 18B). Urelumab is crosslinking independent antibody developed by Bristol Myers Squibb. This antibody shows clinical efficacy, but its development is limited by liver toxicity. FIG. 18A, 1923Ab7, 1923Ab8, 1923Ab17 and 1923Ab18 demonstrated stronger induction (Emax) of PD-L1 dependent CD137 signaling compared to Urelumab.

### Example 19: Characterization of Bispecifics and Trispecifics—Location of scFv of Anti-CD137

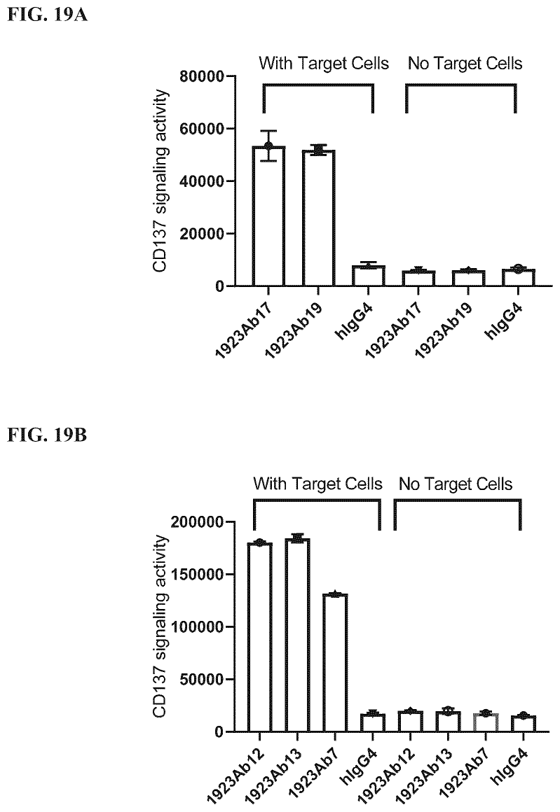

To examine the effect on CD137 agonism by fusing ScFv of anti-CD137 in a different location in the disclosed bispecific and trispecific antibodies, Jurkat T CD137 reporter cells were used in the presence or absence of PDL1 expressing cells. The structure of the testing antibodies are illustrated in FIGS. 13 and 14. 1923Ab19 contains the ScFv format of 1923Ab4 fused at the C-terminus of antibody light chain. 1923Ab12 and 1923Ab13 contains ScFv format of 1923Ab4 fused at the N-terminus of antibody light chain and heavy chain, respectively. 1923Ab17 and 1923Ab7 are control trispecific antibodies. FIG. 19A demonstrates that bi-specific Ab 1923Ab19 can only induce CD137 signaling in presence of PDL1 expressing cells. Similarly, n1923Ab12 and 1923Ab13 also induced CD137 signaling in PD-L1 dependent manner (FIG. 19B).

### Example 20: Characterization of Bispecifics and Trispecifics—Blocking of PD-1/PD-L1 Interaction

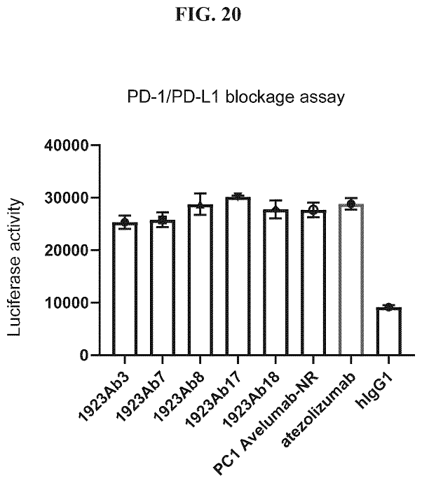

To examine the effect of the PDL1 binding arm in bispecific and trispecific antibodies to block the interaction between PD-1 and PD-L1, a PD-1/PD-L1 inhibition bioassay developed by Promega (Madison, USA) was used as described in Example 4. FIG. 20 demonstrates that all the disclosed antibodies including 1923Ab3, 1923Ab7, 1923Ab8, 1923Ab17 and 1923Ab18 effectively block the interaction between PD-1 and PD-L1. The blockage activity is equivalent to clinically approved anti-PD-L1 antibodies such as PC1 Avelumab-NR and atezolizumab.

### Example 21: Characterization of Trispecifics—Blocking of TGFβ Activity

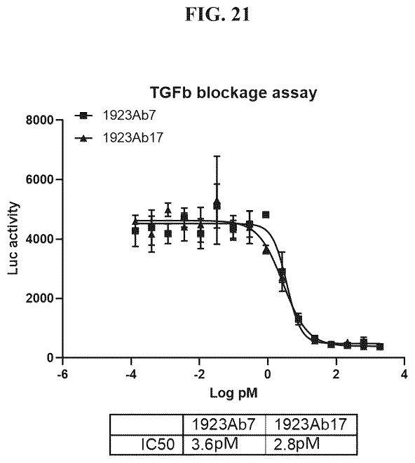

Higher levels of transforming growth factor beta (TGFβ) are associated with immune escape, therapy resistance (chemotherapy, radiation, checkpoint inhibitors), and poor outcomes in advanced malignancies. Inhibiting TGFβ signaling by sequestering TGFβ in the tumor microenvironment (TME) leads to phenotypic changes in nonimmune cells and enhanced activation of immune cells. Therefore, blockage of TGFβ provides benefit to our disclosed bispecific antibodies to fully engage the immune system. To do so, we generated trispecific antibodies such as 1923Ab7 and 1923Ab17. The TGFβ blockage activity of these antibodies was examined using TGF/SMAD Signaling Pathway SBE Reporter—HEK293 Cell Line (BPS Bioscience, CA). FIG. 21 demonstrates that both 1923Ab7 and 1923Ab17 effectively blocked TGFβ-induced signaling with IC50 values of 3.6 pM and 2.8 pM, respectively.

### Example 22: Characterization of Bispecifics and Trispecifics—T Cell Activation Using Human PBMCs

To measure T cell activation, human PBMCs prepared from a healthy donor were used. These human PBMCs were cultured at density of 1×10⁶ cells/mL in RPMI1640 media supplemented with 10% FBS and 0.5 ug/ml of mouse anti-hCD3 clone OKT3 (Biolegend, Cat #: 317325). The disclosed antibodies were added to stimulate T cells. The plate was incubated for 3 days at 37° C. with 5% CO₂. After 72 hours of incubation, the supernatant was used to measure the secreted IFNγ by AlphaLISA (PerkinElmer, Cat #: AL217C/F) using protocols according to the manufacture's instruction.

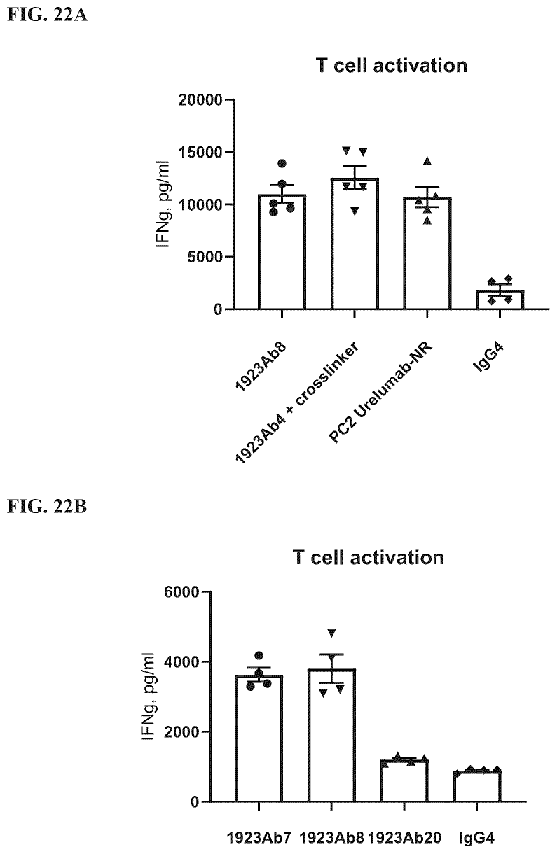

As shown in FIG. 22A, when co-treated with CD3, bispecific Ab 1923Ab8 induced strong T cell activation equivalent to PBMCs treated with either monoclonal anti-CD137 Ab 1923Ab4+Fc-crosslinker or PC2 Urelumab-NR alone. Previous data in example 18 demonstrated the activity of 1923Ab8 is PDL-1 dependent. Since expression of PDL1 on antigen presenting cells in PBMC and activated T cells has been reported, this data suggests the bispecific Ab may be able to bind to PDL1 on immune cells and activate T cells in tumor microenvironment and draining lymph nodes where tumor antigen-experienced T cells exist.

FIG. 22B demonstrates that both trispecific 1923Ab7 and bispecific 1923Ab8 induced strong T cell activation. However, a bispecific Ab 1923Ab20 containing anti-PDL1 and TGFbRII, did not show T cell activation. Blocking PD1 pathway and activating CD137 both can activate T cells. The result illustrates that the detected T cell activation is due to activation of CD137 signaling rather than the inhibition of PD-1/PD-L1 interaction.

### Example 23: Characterization of Bispecifics and Trispecifics—T Cell Mediated Cytotoxicity and CD8 T Cell Activation

To examine the T cell mediated cytotoxicity effect on tumor cells expressing PD-L1, NUGC-4 gastric tumor cells expressing GFP were used. In brief, NUGC4 GFP cells were pre-treated with IFNγ for 48 hours to induce PD-L1 expression. After pre-treatment, the cells were washed and co-cultured with CD8 T cells stimulated with mouse anti-hCD3 clone OKT3 (Biolegend, Cat #: 317325) and the disclosed bispecific or trispecific antibodies for 72 hours. Green fluorescent signal was measured using Cytation (Biotek, VT). The percentage of killing was calculated by the following formula:

% of killing=(GFP signal no antibody−GFP signal treated with antibody)/GFP signal no antibody*100%.

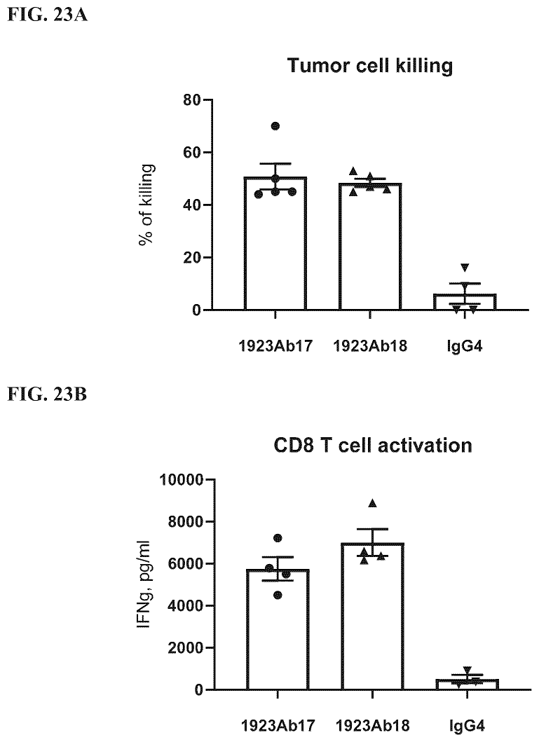

FIG. 23A demonstrates that 1923Ab17 and 1923Ab18 induced strong T cell mediated cytotoxicity. Around 50% of tumor cells were killed upon 72 hours of incubation with CD8 T cells stimulated with 1923Ab17 or 1923Ab18. Media from the same experiment were used to measure the amount of IFNγ as an indication of T cell activation. FIG. 23B demonstrates that both 1923Ab17 and 1923Ab18 treatment induced strong T cell activation compared to isotype control treatment. These results illustrate that 1923Ab17 and 1923Ab18 not only activated CD8 T cells but also induced CD8 T cell mediated killing.

### Example 24: Characterization of Bispecifics and Trispecifics—Antigen Specific T Cell Activation Using PBMCs in the CMV Recall Assay

To examine antigen specific T cell activation of the disclosed bispecific and trispecific antibodies, human PBMCs prepared from a healthy donor were used. CMV lysate was purchased from Microbix Biosystems (Cat #: EL-01-02-001.0). The CMV recall assay was performed using human PBMCs stimulated with CMV lysate and antibodies for 5 days. The plate was incubated at 37° C. with 5% CO₂. After 5 days of incubation, supernatants were collected and IFNγ was measured by AlphaLISA (PerkinElmer, Cat #: AL217C/F) using protocols according to the manufacture's instruction. The amount of IFNγ is directly proportion to T cell activation.

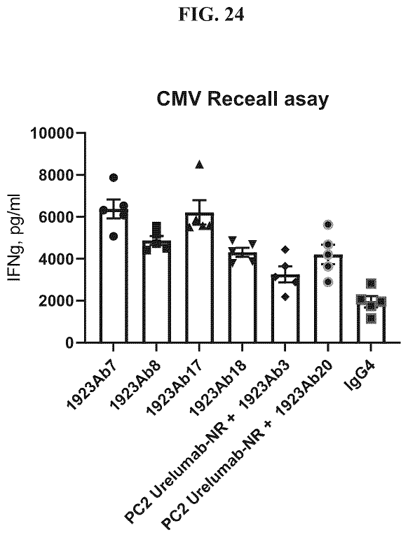

FIG. 24 demonstrates that all the disclosed antibodies activated antigen specific T cell activation. Among all, 1923Ab7 and 1923Ab17 induced the strongest T cell activation in vitro. This result indicates that trispecific antibodies activated antigen specific T cell activation stronger than bispecific antibodies or combination treatment of “PC2 Urelumab-NR+1923Ab3” or “PC2 Urelumab-NR+1923Ab20” did.

### Example 25: Binding Kinetics of 1923Ab17 and 1923Ab18 to Human P-L1 and Human CD137

To assess the ability of the disclosed antibodies, including 1923Ab3, 1923Ab4, 1923Ab17 and 1923Ab18, to bind human PD-L1 and CD137, the binding kinetic experiment was tested by Biacore3000. Briefly, CM5 sensor chip (GE Healthcare, cat #BR-1000-12) was immobilized via amine coupling chemistry with anti-human IgG antibody (GE Healthcare, cat #BR-1008-39) following the application wizards on Flow cell 2. Flow cell 1 remained unmodified to serve as a reference cell for the subtraction of systematic instrument noise and drift. Run Fc2-1 detection with double blank (Fc1 and blank analyte buffer), antibody samples were diluted to 1 ug/ml in HBS-EP (GE Healthcare, cat #BR-1003-69) and injected at flow rate of 10 ul/ml for 1 min capture. Antigens human PD-L1 (Acro Biosystems, cat #PD1-H5229) and CD137 (Sino Biological, cat #10041-H08H) diluted from 10 nM for PD-L1 and 80 nM for CD137 with 1:4 dilution, 5 points to 0 nM in HBS-EP were injected at 50 ul/min for 2 min, followed by 6 min dissociation.

Data was analyzed in BIAEvalution software by 1:1 binding with mass transfer model. the equilibrium dissociation constant (KD) was reported. The results are summarized in TABLES 10 and 11.

TABLE 10
Binding to human PD-L1

| | ka (1/Ms) | kd (1/s) | KD (M) |
|---|---|---|---|
| 1923Ab3 | 8.14E+05 | 2.40E−03 | 2.95E−09 |
| 1923Ab17 | 1.20E+06 | 2.67E−03 | 2.22E−09 |
| 1923Ab18 | 6.59E+05 | 2.30E−03 | 3.49E−09 |

TABLE 11
Binding to human CD137

| | ka (1/Ms) | kd (1/s) | KD (M) |
|---|---|---|---|
| 1923Ab4 | 3.05E+06 | 2.19E−02 | 7.18E−09 |
| 1923Ab17 | 2.50E+06 | 4.83E−02 | 1.93E−08 |
| 1923Ab18 | 1.70E+06 | 3.80E−02 | 2.23E−08 |

### Example 26: Anti-Tumor Effect and Tumor Infiltrated Lymphocyte (TIL) Analysis in a MC38-hPD-L1 Mouse Tumor Model

Female B-hPD-L1/h4-1BB mice (Biocytogen), 6-8 weeks of age, with a body weight between 16-20 g, were acclimated for 7 days prior to study enrollment. Mice had free access to autoclaved sterilized dry granule food and water during the entire study period and were housed on 12 hours light/dark cycle at 20-26° C. with relatively humidity 40-70%. The MC38 murine colon carcinoma cell line was genetically modified to overexpress human PD-L1 instead of mouse PD-L1. Cells are maintained in vitro as a monolayer culture in DMEM supplemented with 10% heat inactivated FBS at 37° C. in an atmosphere of 5%. Cells were harvested and 5×10⁵ cells in 100 μl of PBS were subcutaneously implanted into the right front flank for tumor development. On day 7, tumor-bearing mice will be randomly enrolled into 2 study groups with the mean tumor size approximately 100-150 mm³. Each group consisted of 5 mice. Tumor size will be measured two times weekly in two dimensions using a caliper, and the volume is expressed in mm3 using the formula: V=0.5a×b2 where a and b are the long and short dimensions of the tumor, respectively. Treatment started on days 7, 11, 14 and 18 with an intraperitoneal injection of 5 mg/kg 1923Ab18 or PBS as a negative control. The study was terminated on day 21. Terminal bleeding was performed to prepare serum for AST measurement. Tumors were harvested and stored in the tissue storage buffer for TILs analysis.

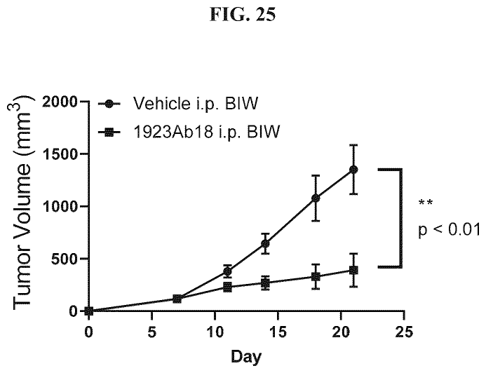

FIG. 25 shows the tumor growth curves for both treatment groups. 1923Ab18 significantly inhibited tumor growth compared to vehicle control. It reached 78% tumor growth inhibition.

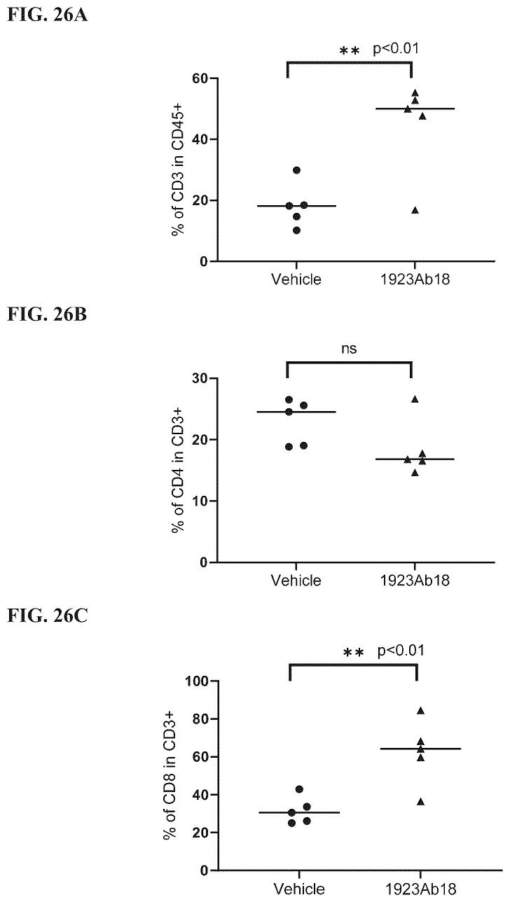

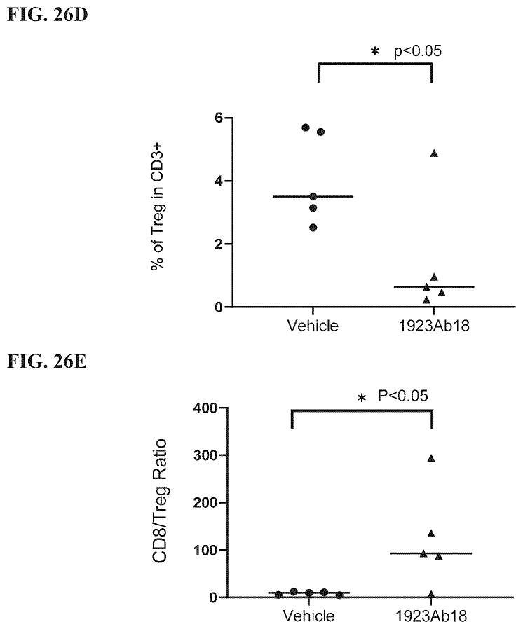

To determine the effect of the 1923Ab18 antibody on immune cells in the tumor microenvironment, tumor samples were harvested in the study described above. Overall infiltration of immune cells was determined by measuring the quantity of CD3+/CD45+ tumor infiltrating lymphocytes. As shown in FIG. 26A, 1923Ab18 significantly increased infiltration of CD3+ immune cells into the tumor microenvironment. The CD3+ cells were further divided into CD4+ or CD8+ TILs. FIGS. 26B & C demonstrates that 1923Ab18 significantly induced infiltration of CD8+ cells but not CD4+ cells. The level of Treg cells in the tumor microenvironment was determined by measuring the quantity of CD25+ FOXP3+ CD3+ tumor infiltrating lymphocytes. As shown in FIG. 26D, 1923Ab18 significantly reduced the level of Tregs in the tumor microenvironment. Therefore, CD8/Treg ratio was significantly increased in the tumor microenvironment of mice treated with 1923Ab18 (FIG. 26E).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1-105. (canceled)

106. An anti-CD137 antibody or antigen binding fragment thereof comprising:
(i) a heavy chain variable region comprising CDR1: SEQ ID NO: 5, CDR2: SEQ ID NO: 22, and CDR3: SEQ ID NO: 23; and a light chain variable region comprising CDR1: SEQ ID NO: 24, CDR2: SEQ ID NO: 25, and CDR3: SEQ ID NO: 26;
(ii) a heavy chain variable region comprising CDR1: SEQ ID NO: 27, CDR2: SEQ ID NO: 28, and CDR3: SEQ ID NO: 29; and a light chain variable region comprising CDR1: SEQ ID NO: 30, CDR2: SEQ ID NO: 9, and CDR3: SEQ ID NO: 31; or
(iii) a heavy chain variable region comprising CDR1: SEQ ID NO: 32, CDR2: SEQ ID NO: 33, and CDR3: SEQ ID NO: 34; and a light chain variable region comprising CDR1: SEQ ID NO: 35, CDR2: SEQ ID NO: 36, and CDR3: SEQ ID NO: 37.

107. The anti-CD137 antibody or antigen binding fragment thereof of claim 106, wherein the anti-CD137 antibody or antigen binding fragment thereof comprises:
(i) a heavy chain variable region comprising SEQ ID NO: 16; and a light chain variable region comprising SEQ ID NO: 17;
(ii) a heavy chain variable region comprising SEQ ID NO: 18; and a light chain variable region comprising SEQ ID NO: 19; or
(iii) a heavy chain variable region comprising SEQ ID NO: 20; and a light chain variable region comprising SEQ ID NO: 21.

108. The anti-CD137 antibody or antigen binding fragment thereof of claim 106, wherein the binding protein or binding fragment thereof comprises a heavy chain constant region.

109. The anti-CD137 antibody or antigen binding fragment thereof of claim 108, wherein the constant region comprises SEQ ID NO: 60, SEQ ID NO: 61, SEQ ID NO: 62, SEQ ID NO: 63, or SEQ ID NO: 64.

110. The anti-CD137 antibody of claim 106, wherein the anti-CD137 antibody comprises a heavy chain comprising SEQ ID NO: 75; and a light chain comprising SEQ ID NO: 76.

111. The anti-CD137 antibody or antigen binding fragment thereof of claim 106 comprising a light chain constant region comprising SEQ ID NO: 65 or SEQ ID NO: 66.

112. The anti-CD137 antibody of claim 106, wherein the anti-CD137 antibody is a multi-specific antibody, preferably a bispecific or tri-specific antibody.

113. The antigen binding fragment thereof of claim 106, wherein the binding fragment thereof is a Fab, a Fab′, a F(ab)₂, a F(ab′)₂, a Fd, a Fv, a scFv, a single-chain antibody, a minibody, or a diabody.

114. An anti-PD-L1 antibody or antigen binding fragment thereof comprising:
(i) a heavy chain variable region comprising CDR1: SEQ ID NO: 5, CDR2: SEQ ID NO: 6, and CDR3: SEQ ID NO: 7; and a light chain variable region comprising CDR1: SEQ ID NO: 8, CDR2: SEQ ID NO: 9, and CDR3: SEQ ID NO: 10; or
(ii) a heavy chain variable region comprising CDR1: SEQ ID NO: 11, CDR2: SEQ ID NO: 12, and CDR3: SEQ ID NO: 13; and a light chain variable region comprising CDR1: SEQ ID NO: 14, CDR2: SEQ ID NO: 9, and CDR3: SEQ ID NO: 15.

115. A nucleic acid composition comprising one or more nucleic acids encoding the antibody or antigen binding fragment thereof of claim 106.

116. An expression vector composition comprising one or more expression vectors comprising the nucleic acid composition of claim 115.

117. A host cell comprising the expression vector composition of claim 116.

118. A method for the production of an anti-CD137 antibody or antigen binding fragment thereof, the method comprising culturing the host cell of claim 117.

119. A pharmaceutical composition comprising the anti-CD137 antibody or antigen binding fragment thereof of claim 106 and a pharmaceutically acceptable carrier.

120. A method of treating cancer in a subject, the method comprising administering the anti-CD137 antibody or antigen binding fragment thereof of claim 106 to the subject.
